(19) United States
(12) Patent Application Publication
(10) Pub. No.: US 2025/0231188 A1
(43) Pub. Date: Jul. 17, 2025

(54) COMPOSITIONS FOR SIALIC ACID SENSING, CANCER CELL IMAGING, AND METHODS OF USE THEREOF

(71) Applicant: The University of Hong Kong, Hong Kong (CN)

(72) Inventors: Vivian Wing-Wah Yam, Hong Kong (CN); Jungu Guo, Hong Kong (CN); Sin-Yee Law, Hong Kong (CN)

(21) Appl. No.: 19/017,095

(22) Filed: Jan. 10, 2025

Related U.S. Application Data
(60) Provisional application No. 63/620,355, filed on Jan. 12, 2024.

(51) Int. Cl. G01N 33/574; G01N 33/53; G01N 33/543; G01N 33/553

(52) U.S. Cl. CPC G01N 33/57492; G01N 33/5308; G01N 33/54313; G01N 33/553

(57) ABSTRACT

Compounds, particularly compounds capable of sensing and/or imaging glycans (such as sialic acids, e.g., mono-sialic acids, disialosides, trisialosides, polysialic acids), and/or cancer cells, and differentiating cancer cells from normal cells, are disclosed. The compounds are d⁸ or d¹⁰ metal complexes or salts thereof. The metal complexes can bind to glycans (such as sialic acids, e.g., mono-sialic acids, disialosides, trisialosides, polysialic acids), and/or cancer cells. The binding interaction induces accumulation and supramolecular self-assembly of the metal complexes, thereby causing changes in the photophysical properties of the metal complexes.

### CROSS-REFERENCE TO RELATED APPLICATIONS

This application claims the benefit of and priority to U.S. Provisional Application No. 63/620,355 filed Jan. 12, 2024, which is herein incorporated by reference in its entirety.

### FIELD OF THE INVENTION

The invention is in the field of early diagnosis and imaging guidance therapies, particularly detecting and/or sensing analytes, especially glycans (such as sialic acids, e.g., mono-sialic acids, disialosides, trisialosides, etc., and polysialic acids); and/or cancer cells as well as visualizing and monitoring glycans (such as sialic acids and polysialic acids) in biological samples and differentiating cancer cells from normal cells.

### BACKGROUND OF THE INVENTION

All eukaryotic cells are covered with a dense and complicated layer of glycans. Glycan plays an essential role in regulating many cellular physiological and pathological processes, including cell trafficking and signaling via transmitting information among cells in the form of glycoproteins and glycolipids (Dube et al., Nat. Rev. Drug Discov., 4, 477-488 (2005)). Glycans possess structural diversity, each of which has unique structures with varying types of monosaccharide sequences (Laughlin, et al., Proc. Natl. Acad. Sci. U.S.A., 106 (1), 12-17 (2009)). Sialic acids share a nine-carbon backbone, and they are typically located at the terminal ends of the glycan chain on the cell surfaces (Schauer et al., Glycoconj. J., 17, 485-499 (2000)). Sialic acids modulate various biological processes, contributing to signal transduction and immune response. An intriguing physiological role of sialic acids is to transmit a “self” signal to immune cells so that cells can escape the attack from the immune system. The increased level of sialic acids in many cancer cells is significant in acting as a biological mask to evade recognition from the immune system and facilitate metastatic spread (Narayanan et al., Ann. Clin. Lab. Sci., 24 (4), 376-384 (1994)). The overexpressed sialic acids can be utilized as a biomarker for cancer. Thus, developing sensitive and selective sensors to detect sialic acids, polysialic acids, glycans, and cancer cells is of great importance in early diagnosis and therapeutic guidance.

Quantification methods of glycans such as sialic acids, polysialic acids, etc., in samples (preferably biological samples such as serum or plasma) have been established to facilitate the exploration of further mechanisms related to psychological and pathological processes and early diagnosis. The common methods include high-performance liquid chromatography (HPLC), mass spectrometry (MS), and colorimetric assay (Zhou et al., Cells, 9 (2), 273-291 (2020)). These assays are usually used to detect sialic acids in vitro, but it is difficult to provide in vivo dynamic information during physiological and pathological processes, limiting their further application. Bio-imaging can visualize biological species in live cells and facilitate diagnosis and therapy, attracting tremendous attention.

The reported recognition strategies for glycans, such as sialic acids, polysialic acids, etc., involves bio-affinity, chemical, metabolic, and linkage labeling (Xiong et al., Polymers, 9 (7), 249-266 (2017)). The most widely used recognition group is phenylboronic acid (PBA) moiety, which can form a five- or six-membered cyclic ester with the diol moiety of sialic acids. The ester formed is more stable than other PBA-sugar esters in the cell environment. Thus, PBA has been introduced into nanoparticles, metal oxide-coated electrodes, organic fluorophores, and lanthanide chelators to afford the sensors (Wang, et al., Nanoscale, 10, 4570-4578 (2018)). For example, Lo and co-workers incorporated the PBA group into the cyclometalated iridium(III) bipyridine complex to recognize cellular sialic acid residues and discriminate between cancer and normal cells (Liu, et al., Chem.-Asian J., 12, 1545-1556 (2017)). However, the low selectivity resulting from the binding interaction between PBA and diol moiety of other monosaccharides affects the accuracy of the sensors. Besides, the reported probes often suffer from false positives because of a minute amount of sialic acids existing on the surface of normal cells. New and/or improved recognition moieties for sialic acid and new and/or improved detecting strategies are in urgent need to improve selectivity and avoid false-positive results.

It is the aim of the present invention to offer compounds to sense sialic acids and/or light up cancer cells, particularly to (1) detect and visualize sialic acids, polysialic acids, and glycans; (2) differentiate cancer cells from normal cells; (3) screen/test the efficiency of inhibitors to remove sialic acids for therapies.

It is another aim of the present invention to develop methods to image sialic acids and/or light up cancer cells, particularly to (1) detect and visualize glycans, such as sialic acids, polysialic acids, etc.; (2) differentiate cancer cells from normal cells; (3) screen/test the efficiency of inhibitors to remove sialic acids for therapies.

It is yet another aim of the present invention to produce kits to image sialic acids and/or light up cancer cells, particularly to (1) detect and visualize glycans, such as sialic acids, polysialic acids, etc.; (2) differentiate cancer cells from normal cells; (3) screen/test the efficiency of inhibitors to remove sialic acids for therapies.

### SUMMARY OF THE INVENTION

Disclosed are compounds, mixtures, methods, and kits for sensing and/or imaging an analyte, and/or lighting up cells, and/or screening and/or testing inhibitors, and/or early diagnosis and imaging guidance.

In this application, some examples of compounds will be provided: in some forms, the compounds can be d⁸ or d¹⁰ metal complexes. The versatile coordination modes contain square-planar geometry of d⁸ metal complexes, and trigonal-planar and linear geometries of d¹⁰ metal complexes. The compounds are generally composed of at least one metal center and at least one coordinating ligand:
(a) one or more metal centers with a coordination number of 2, 3, or 4, selected from Pt(II), Pd(II), Ni(II), Ir(I), Rh(I), Au(III), Ag(III), Cu(III), Ni(O), Pd(O), Pt(O), Cu(I), Ag(I), Au(I), Zn(II), Cd(II), and Hg(II), and
(b) one or more ligands with donor atoms selected from the group containing carbon (C), nitrogen (N), oxygen (O), phosphorus (P), sulfur (S), arsenic (As), and selenium (Se).

The formed metal complexes can have a planar structure, partially planar, or linear structures. Noncovalent interactions, including electrostatic interactions, hydrogen bonding interactions, hydrophobic interaction, and combinations thereof, can facilitate the binding of the metal complexes to analytes, leading to supramolecular self-assembly of the bound metal complexes via noncovalent metal-metal interactions and/or π-π stacking interactions. The electrostatically and/or noncovalently bound metal complex-analyte adducts or ensembles are further stabilized by the noncovalent metal-metal interactions and/or π-π stacking interactions arising from the self-assembly and/or aggregation of the metal complexes and are not easily disrupted by non-specific interactions. The supramolecular self-assembly of compounds can be ascribed to their planar, partially planar, or linear geometries. In some forms, the analyte can be glycans, such as sialic acids, polysialic acids, etc.; or cancer cells. Glycans, including sialic acids, are found on the surfaces of cells and play a crucial role in cell trafficking and signaling during physiological processes. In the case of cancer cells, they must evade detection by the immune system and promote metastatic spread, leading to an increase in the levels of sialic acids. Such disparity in glycan levels between cancer cells and normal cells can be exploited to distinguish different types of cells. Compounds that contain positively charged groups and/or amino acids, along with their derivatives, or combinations of amino acids and/or their derivatives, have the ability to bind sialic acids and polysialic acids. Additionally, these compounds possess self-assembly capabilities, and their associated photophysical properties can be easily probed due to the presence of metal centers.

The self-assembly and aggregation of the metal complexes upon addition of the analytes can lead to remarkable changes in the photophysical properties of the metal complexes. In some forms, the photophysical properties include absorption and luminescence. In some forms, the changes in luminescence can be a red shift, or a blue shift of emission wavelength, or emission intensity. The changes in the photophysical properties could be utilized as luminescence signals to detect analytes.

In some forms, noncovalent interactions can contribute to the aggregation of the metal complexes. These noncovalent interactions include metal-metal interactions and/or π-π stacking interactions, electrostatic interactions, hydrogen bonding interactions, hydrophobic interactions, and combinations thereof. The formed complex-analyte ensemble resulting from noncovalent interactions endows the metal complexes with the ability to assemble in close proximity and form aggregates, giving rise to changes in the photophysical properties, such as the luminescence of the metal complexes.

The specificity of the metal complexes toward a specific analyte can be attributed to one or more combinations of noncovalent interactions. As shown in the description and the examples, design strategies can be utilized to introduce functional groups for different types of noncovalent interactions between the metal complexes and the analyte to improve the specificity. The square-planar, partially planar, trigonal planar, or linear geometry endows d⁸ or d¹⁰ complexes with the ability to stack with each other, leading to the formation of aggregates. The subsequent spectral changes can be used for the detection of analytes.

The introduction of moieties which can bind to the analyte via noncovalent interactions endows the metal complexes with high selectivity and specificity toward the analyte of interest. The presence of one or more functional groups may facilitate higher binding affinities with the help of a combination of different kinds of noncovalent interactions.

Preferably, the analyte contains repeating monomers, which are beneficial for the aggregation and self-assembly of the metal complexes upon binding to the analyte.

Disclosed are seven types of chemical formulae (Formula I-VII) for detecting and/or visualizing an analyte, wherein the metal complexes contain one or more functional groups binding to the analyte through electrostatic interactions and hydrogen bonding. The specific interactions-induced supramolecular self-assembly of the metal complexes result in a change of luminescence, which can be utilized for detection.

The compounds have the chemical structure shown in the generic Formula I:

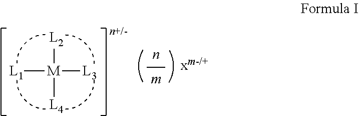

### Wherein:

(a) M is a metal atom (also known as metal center). It can be Pt(II), Pd(II), Ni(II), Ir(I), Rh(I), Au(III), Ag(III), and Cu(III);
(b) L₁, L₂, L₃, L₄ represent ligands. Each ligand can offer one or more donor atoms, preferably one donor atom, for coordination to the metal center;
(c) n+/− is the number of charge (positive or negative) that the metal complexes carry. n can be zero or a positive integer, such as 1, 2, 3, 4, and 5;
(d) X is a counterion for charge neutrality of the compound. When X^{m-/+} is an anion, for example, X^{m-}, it can be selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof. When X^{m-/+} is a cation, for example, X^{m+}, it can be selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof;
(e) m−/+ is the number of charge (negative or positive) that the counterions carry. X should carry the charge opposite to the charge of the metal complex. m can be zero or a positive integer, such as 1, 2, 3, 4, and 5. And wherein m=n or m≠n;
(f) n/m represents the stoichiometry of the counterions in the formula; and
(g) Four dashed lines represent the optional independent covalent linking between two ligands, optional fusion of ring moieties from two ligands, or a combination thereof.

In some forms, the metal complex has a chemical structure shown by a generic Formula II:

$${[ {L}_{5} — {M}^{′} — {L}_{6} ]}^{n + / -} ( \frac{n}{m} ) {X}^{m - / +} \qquad Formula II$$

### Wherein:

(a) M′ is a metal center selected from Ni(0), Pd(0), Pt(0), Cu(I), Ag(I), Au(I), Zn(II), Cd(II) and Hg(II).
(b) L₅ and L₆ represent ligands. Each ligand can offer at least one donor atom, preferably one donor atom, for coordination to the metal center.

In some forms, the metal complex has a chemical structure shown by a generic Formula III, which exhibit a trigonal-planar geometry with monodentate, bidentate, tridentate ligands:

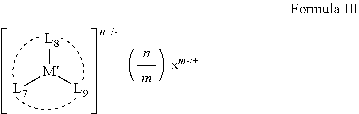

(a) L₇, L₈, and L₉ stand for ligands. Each ligand can offer at least one donor atom, preferably one donor atom, for coordination to the metal center.

In some forms, the ligand has a chemical structure shown by a generic Formula IV:

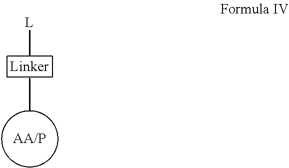

### Wherein:

(a) L stands for chemical moieties containing one or more donor atoms, preferably one donor atom, for coordination to the metal center of the metal complexes;
(b) Linker stands for structures which can be used as the optional covalent linking moieties between L and amino acids/positive charge-containing structures;
(c) AA stands for amino acids, or their derivatives, or combination of amino acids and/or their derivatives, and
(d) P represents positive charge-containing structure(s). The structures bearing positive charge can be selected from, but are not limited to, substituted and unsubstituted amine, ammonium, pyridinium, pyrrodinium, phosphonium, imidazolium, sulfonium, and their derivatives.

Also disclosed are methods for making exemplary compounds. The methods can be used in the range of a wide variety of functional groups, ligands, metal complexes, and compounds, and then various derivatives can be obtained using the disclosed methods.

Also disclosed are methods for detecting glycans, such as sialic acids polysialic acids; and/or cancer cells. The procedures of the assay method are listed as follows: (a) combining a sample (preferably a biological sample) containing an analyte with one or more disclosed compounds; (b) measuring the changes of the photophysical properties of the metal complexes. The assembly/disassembly is indicated by the variation of the photophysical properties. Noncovalent interactions, such as electrostatic interactions and hydrogen bonding, play a huge role in the binding interaction between metal complexes and biological analytes. This binding may induce changes in the photophysical properties of the metal complexes, which can be attributed to the different electron-donating or electron-withdrawing ability of certain functional groups present in metal complexes. However, these changes can be unpredictable and slight changes in molecular state may affect the detection efficiency, which is the limitation of traditional sensing strategies.

As a new and improved sensing strategy, the self-assembly and aggregation of metal complexes can result in significant changes in their photophysical properties, providing a method for bio-imaging and early diagnosis. (Reference: Liu, et al., Chem.-Asian J., 12, 1545-1556 (2017); Wang, et al., J. Mat. Chem. B, 9, 4690-4699 (2021); Pancera, et al., Nat. Struct, Mol. Biol., 20, 804-814 (2013)). The changes of photophysical properties indicate the presence of glycans, such as sialic acids, polysialic acids; and/or cancer cells.

Also described are methods for imaging glycans, such as sialic acids, polysialic acids, etc.; and cancer cells; and differentiating cancer cells from normal cells. The procedures are listed as follows: (a) combining one or more disclosed compounds with a sample (preferably a biological sample) under conditions to allow for binding of the metal complexes with a high level of glycans, such as sialic acids, polysialic acids, etc. The number of sialic acid molecules on the cellular surface of cancer cells can range from 10⁴-10¹¹, 10⁵-10¹¹, 10⁶-10¹¹, or 10⁷-10¹¹ molecules, particularly 10⁷-10¹¹ molecules, which can be regarded as a high level. The reported luminescent assays have reported higher levels of sialic acids in cancer cells compared to normal cells. The high density of these sialic acids leads to the self-assembly and aggregation of compounds that target them in cancer cells (Wang, et al., Anal. Chem., 2017, 89, 538-543; Xu, et al., Talanta, 2020, 209, 120579). The subsequent assembly and/or aggregation of the metal complexes induces changes of photophysical properties; (b) imaging glycans, such as sialic acids, polysialic acids, etc., on the surface of cells based on variation of one or more photophysical properties; (c) differentiating cancer cells from normal cells based on different luminescence signals. Glycans, including sialic acids, are found on the surfaces of cells and play a crucial role in cell trafficking and signaling during physiological processes. In the case of cancer cells, they must evade detection by the immune system and promote metastatic spread, leading to an increase in the levels of sialic acids. Such disparity in glycan levels between cancer cells and normal cells can be exploited to distinguish different types of cells. Compounds that contain positively charged groups and/or amino acids, along with their derivatives, or a combination of amino acids and/or their derivatives have the ability to bind sialic acids and polysialic acids. Additionally, these compounds possess self-assembly capabilities, and their associated photophysical properties can be easily probed due to the presence of metal centers.

Also described are methods for testing the efficacy of inhibitors to remove glycans, such as sialic acids, polysialic acids, etc., from cellular surface(s). The procedures are listed as follows: (a) combining one or more disclosed compounds with the inhibitor-treated sample (preferably a biological sample) and, separately, with an untreated sample (preferably a biological sample) as a control group; (b) comparing the photophysical properties of the metal complexes between two samples. Aggregation and supramolecular self-assembly of the metal complexes generate changes in the photophysical properties of the metal complexes.

In some forms, the sample can be a human or non-human animal body fluid, a human or non-human animal tissue, or a combination thereof. The body fluid can be cerebrospinal fluid; the tissue can be brain tissue. In some forms, the sample can be eukaryotic cells. The cells can be, but not limited to, HeLa cells, Hep G2 cells, HEK293T cells, Chinese hamster ovary (CHO) cells, 3T3 cells, A549 cells, and HT1080 cells.

Also described is a kit for use in detecting and/or imaging glycans (such as sialic acids, polysialic acids, etc.) for use in screening or testing the efficacy of inhibitors that can remove glycans (such as sialic acids, polysialic acids, etc.) from cancer cells. The kits can contain, in one or more containers, one or more of the disclosed compounds and optionally instructions for use. The kits can also contain a carrier.

### BRIEF DESCRIPTION OF THE DRAWINGS

The accompanying drawings, which are incorporated in and constitute a part of this specification, illustrate several embodiments of the disclosed compounds, mixtures, compositions, kits, and methods, and together with the description, serve to explain the principles of the disclosed compounds, mixtures, compositions, kits, and methods.

FIG. 1 shows a cationic d⁸ metal complex (complex 1-Pt) as an illustrative example.

FIG. 2 shows a synthetic route for complex 1-Pt shown in FIG. 1.

FIG. 3 shows the UV-vis absorption spectra of complex 1-Pt (30 μM) upon addition of different amounts of polysialic acids (0-60 μM) in Tris-HCl buffer (10 mM Tris, 10 mM NaCl, pH=8.0).

FIG. 4 shows the UV-vis absorption spectra of complex 1-Pt (30 μM) upon addition of different amounts of polysialic acids (60-114 μM) in Tris-HCl buffer (10 mM Tris, 10 mM NaCl, pH=8.0).

FIG. 5 shows the emission spectra of complex 1-Pt (30 μM) upon addition of different amounts of polysialic acids (0-90 μm) in Tris-HCl buffer (10 mM Tris, 10 mM NaCl, pH=8.0).

FIG. 6 shows a plot of the relative emission intensity at 760 nm versus the concentration of polysialic acids.

FIG. 7 shows the corrected emission spectra of complex 1-Pt (30 μM) upon addition of different amounts of sialic acid (Neu5Ac) (0-90 PM) in Tris-HCl buffer (10 mM Tris, 10 mM NaCl, pH=8.0). The arrows demonstrate the trends of spectral changes.

FIG. 8 shows a bar chart demonstrating the relative emission intensity at 760 nm of a mixture of complex 1-Pt (30 μM) and different monosaccharides (0, 30, 150, 300 μM), polysialic acids (0, 30, 60, 90 μM) in Tris-HCl buffer (10 mM Tris, 10 mM NaCl, pH=8.0). Electrospray ionization mass spectrometry (ESI-MS) results have shown that NEU5AC can bind to the compounds. The data reveal that monomeric NEU5AC does not induce changes in photophysical properties. This can be attributed to the fact that the monomers are unable to bring compounds into proximity, and therefore fail to trigger self-assembly and aggregation. In the presence of sialic acids on branched glycans, the amounts of monosaccharides (sialic acids) rather than their position, can affect the changes in photophysical properties. In the case of other monosaccharides other than sialic acids, selectivity assays have shown that they cannot bind to compounds, thus providing good selectivity.

FIGS. 9A-9J show the luminescence confocal images of live HeLa cells stained with complex 1-Pt of different concentrations of 10 μM (FIGS. 9A-9C), 20 μM (FIGS. 9D-9F), 30 μM (FIGS. 9G-9I) at 37° C. for 1 h. (FIGS. 9A, 9E, and 9H) bright-field; (FIGS. 9B, 9E, and 9H) luminescence confocal images with emission collected at 700-800 nm; (FIGS. 9C, 9F, and 9I) merged confocal images with bright-field and emission collected at 700-800 nm; (FIG. 9J) is a bar graph comparing relative emission intensity between the different concentrations.

FIGS. 10A-10P show the luminescence confocal images of live HepG2 cells incubated with complex 1-Pt (10 μM) at 37° C. for 0.5 h (FIGS. 10A, 10F, and 10K), 1 h (FIGS. 10B, 10G, and 10L), 2 h (FIGS. 10C, 10H, and 10M), 6 h (FIGS. 10D, 10I, and 10N), 8 h (FIGS. 10E, 10J, and 10O). (FIGS. 10A-10E) Luminescence confocal images with emission collected at 700-800 nm; (FIGS. 10F-10J) bright-field; (FIGS. 10K-10O) merged confocal images with bright-field and emission collected at 700-800 nm; (FIG. 10P) is a bar graph comparing relative emission intensity of complex 1-Pt overtime for 0.5 h, 2 h, 6 h, and 8 h.

FIGS. 11A-11G shows the luminescence confocal images of live HepG2 cells (FIGS. 11A-11C) and live HEK293T cells (FIGS. 11D-11F) stained with complex 1-Pt (10 μM) at 37° C. for 0.5 h. (FIGS. 11A, 11D) Luminescence confocal images with emission collected at 700-800 nm; (FIGS. 11B, 11E) bright-field; (FIGS. 11C, 11F) merged confocal images with bright-field and emission collected at 700-800 nm; (FIG. 11G) is a bar graph comparing relative emission intensity between the HepG2 cells and HEK293T cells stained with complex 1-Pt.

FIGS. 12A-12G show the luminescence confocal images of live HeLa cells (FIGS. 12A-12C) and live HEK293T cells (FIGS. 12D-12F) stained with complex 1-Pt (10 μM) at 37° C. for 0.5 h. (FIGS. 12A, 12D) Luminescence confocal images with emission collected at 700-800 nm; (FIGS. 12B, 12E) bright-field; (FIGS. 12C, 12F) merged confocal images with bright-field and emission collected at 700-800 nm; (FIG. 12G) bar graph comparing relative emission intensity between the HeLa cells and HEK293T cells stained with complex 1-Pt.

FIGS. 13A-13D show the luminescence confocal images of live HepG2 cells (FIGS. 13A-13D) stained with complex 1-Pt (10 μM) for 0.5 h, followed by incubation with paraformaldehyde fix solution for 15 mins and FITC-conjugated lectins (20 μg/mL) for 1 h. (FIG. 13A) Luminescence confocal image with emission collected at 700-800 nm with an excitation wavelength of 405 nm; (FIG. 13B) luminescence confocal image with emission collected at 500-550 nm with an excitation wavelength of 488 nm; (FIG. 13C) merged confocal images with emission collected at 700-800 nm and 500-550 nm; (FIG. 13D) the intensity curve of (FIG. 13A) and (FIG. 13B); (FIG. 13E) line graph shows overlapping luminescence signals of complex 1-Pt and commercial dye with the marked line (-) in FIG. 13C

FIGS. 14A-14E show the luminescence confocal images of live HepG2 cells stained with complex 1-Pt (10 μM) for 0.5 h, followed by incubation of membrane tracker (5 μg/mL) for 10 minutes. (FIG. 14A) Luminescence confocal image with emission collected at 700-800 nm with an excitation wavelength of 405 nm; (FIG. 14B) luminescence confocal image with emission collected at 650-670 nm with an excitation wavelength of 635 nm; (FIG. 14C) bright-field; (FIG. 14D) merged confocal images with emission collected at 700-800 nm and 650-670 nm; (FIG. 14E) line graph shows overlapping luminescence signals of live HepG2 cells stained with complex 1-Pt vs commercial dye with the marked line (-) in FIG. 14D.

FIGS. 15A-15G show the luminescence confocal images of live HepG2 cells treated with the neuraminidase for 1 h (FIGS. 15D-15F) and without the neuraminidase (FIGS. 15A-15C). (FIG. 15A and FIG. 15D) Luminescence confocal images with emission collected at 700-800 nm; (FIGS. 15B and 15E) bright-field; (FIG. 15C and FIG. 15F) merged confocal images of bright-field and emission collected at 700-800 nm with an excitation wavelength of 405 nm; (FIG. 15G) bar graph comparing relative emission intensity between the HepG2 cells treated with and without neuraminidase.

FIG. 16 is a bar chart demonstrating the cell viability of HepG2 cells incubated with different concentrations of complex 1-Pt (0.78, 1.56, 3.13, 6.25, 12.5, 25, 50, 100 μM) after incubation at 37° C. for 24 h.

FIGS. 17A-17E show the luminescence confocal images of live HEK293T cells stained with complex 1-Pt (10 μM) for 0.5 h, followed by incubation with paraformaldehyde fix solution for 15 mins and FITC-conjugated lectins (20 μg/mL) for 1 h. (FIG. 17A) Luminescence confocal image with emission collected at 700-800 nm with excitation wavelength at 405 nm; (FIG. 17B) luminescence confocal image with emission collected at 500-550 nm with excitation wavelength at 488 nm; (FIG. 17C) merged confocal images with emission collected at 700-800 nm and 500-550 nm; (FIG. 17D) bright-field; (FIG. 17E) line graph shows overlapping luminescence signals of complex 1-Pt and commercial dye with the marked line (-) in FIG. 17C.

FIGS. 18A-18C show the luminescence confocal images of (FIG. 18A) live HepG2 cells and (FIG. 18B) live HEK293T cells stained with complex 9-Pt (10 μM) at 37° C. for 0.5 h. (FIGS. 18A-18B) Merged confocal images with bright-field and emission collected at 700-800 nm; (FIG. 18C) bar graph comparing relative emission intensity between the HepG2 and HEK293T stained with complex 9-Pt.

FIGS. 19A-19C show the luminescence confocal images of (FIG. 19A) live HepG2 cells and (FIG. 19B) live HEK293T cells stained with complex 10-Pt (10 μM) at 37° C. for 0.5 h. (FIGS. 19A-19B) Merged confocal images with bright-field and emission collected at 700-800 nm; (FIG. 19C) bar graph comparing relative emission intensity between the HepG2 cells and HEK293T cells stained with complex 10-Pt.

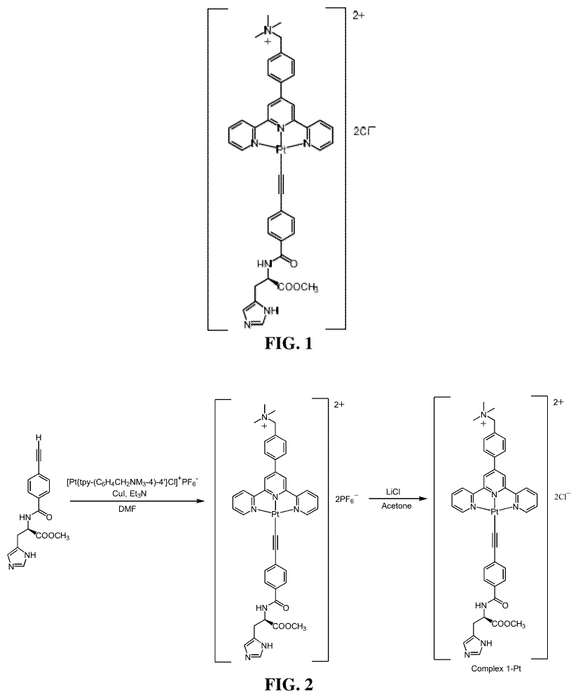

### DETAILED DESCRIPTION OF THE INVENTION

Disclosed are compounds, compositions, methods, and kits for detecting and/or visualizing an analyte and/or screening inhibitors, in particular, for (1) detecting glycans, such as sialic acids, polysialic acids, etc., in a buffer, an aqueous environment, a mixture of aqueous-organic solvent environment, or in any other medium; (2) visualizing high levels of glycans, such as sialic acids, polysialic acids, etc., on the surface of cancer cells with a concentration ranging from 10⁴-10¹¹, 10⁵-10¹¹, 10⁶-10¹¹, or 10⁷-10¹¹ molecules, particularly 10⁷-10¹¹ molecules, which can be regarded as a high level. The reported luminescent assays have reported higher levels of sialic acids in cancer cells compared to normal cells. The high density of these sialic acids leads to the self-assembly and aggregation of compounds that target them in cancer cells. (Wang, et al., Anal. Chem., 2017, 89, 538-543; Xu, et al., Talanta, 2020, 209, 120579); (3) discriminating cancer cells from normal cells for detection or diagnosis of signs of cancer; and/or (4) screening and/or testing the efficacy of inhibitors to remove sialic acids on the surface of cancer cells for anticancer therapies.

In some forms, the compounds include a d⁸ or d¹⁰ metal complex that can bind to an analyte. The analyte can be glycans, such as sialic acids, polysialic acids etc., or cancer cells. The binding can produce a luminescence signal in the red to near-infrared (NIR) region via the aggregation and supramolecular self-assembly of the metal complex through noncovalent metal-metal and/or π-π interactions. Noncovalent interactions including π-π stacking interactions, electrostatic interactions, hydrogen bonding interactions, hydrophobic interactions, and combinations thereof can facilitate the binding between the metal complexes and the analytes, inducing the supramolecular self-assembly and/or aggregation of the metal complexes. The electrostatically and/or noncovalently bound metal complex-analyte adducts or ensembles are further stabilized by the noncovalent metal-metal interactions and/or π-π stacking interactions arising from the self-assembly and/or aggregation of the metal complexes and are not easily disrupted by non-specific interactions. Accompanied by excitation wavelength in the visible-light region and a large Stokes shift, interference resulting from autofluorescence commonly encountered in the presence of various biological substrates can be reduced, rendering the compounds suitable for bioassays.

The disclosed compounds, compositions, kits, and methods can be understood more readily by reference to the following detailed description of particular embodiments and the examples included therein and to the drawings and their previous and following description. All methods described herein can be performed in any suitable order unless otherwise indicated or otherwise clearly contradicted by context.

The use of any and all examples or exemplary language (e.g., “such as”) provided herein, is intended merely to better illustrate the invention and does not pose a limitation on the scope of the invention unless otherwise claimed. No language in the specification should be construed as indicating any non-claimed element as essential to the practice of the invention.

The disclosed compounds, compositions, and kits, can be used for, can be used in conjunction with, can be used in preparation for, or are products of the disclosed methods. It is understood that when combinations, subsets, interactions, groups, etc. of these compounds, compositions, and kits are disclosed, while specific reference of each various individual and collective combinations of these materials may not be explicitly disclosed, each is specifically contemplated and described herein. For example, if a compound is disclosed and discussed and a number of modifications that can be made to a number of molecules including the compound are discussed, each and every combination and permutation of the compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the compounds, compositions, kits, components, etc. contemplated and disclosed as above can also be specifically and independently included or excluded from any group, sub-group, list, set, etc. of such materials. These concepts apply to all aspects of this application including, but not limited to, steps in methods of making and using the disclosed compounds, compositions, and kits. Thus, if there are a variety of additional steps that can be performed, it is understood that each of these additional steps can be performed with any specific embodiment or combination of embodiments of the disclosed methods, and that each such combination is specifically contemplated and should be considered disclosed.

Throughout the description and claims of this specification, the word “include” and variations of the word, such as “including” mean “including but not limited to” and are not intended to exclude, for example, other additives, components, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### I. Definitions

In this application, the following terms should be explained according to the meaning listed below except those separately provided:

The articles “a”, “an”, “the” include plural reference unless the context clearly indicates otherwise. For example, “a complex” includes a plurality of complexes and reference to the compound” is a reference to one or more compounds and equivalents thereof known to those skilled in the art.

The term “can”, “can be”, “may”, and “should”, and relevant terms can be used to indicate that the subject matter involved is optional (that is, the subject matter is present in some embodiments and is not present in other embodiments), not a reference to a capability of the subject matter or to a probability, unless the context clearly indicates otherwise.

The term “optional” and “optionally” can be used to indicate that the subsequently described event, circumstance, or material may or may not occur or be present, and that the description includes instances where the event, circumstance, or material occurs or is present and instances where it does not occur or is not present.

The term “about” can be used to describe values either above or below the stated value in a range of approximately +/ [symbol] 10%; in other embodiments the values may range in value either above or below the stated value in a range of approximately +/ [symbol] 5%; in other embodiments the values may range in value either above or below the stated value in a range of approximately +/−2%; in other embodiments the values may range in value either above or below the stated value in a range of approximately +/ [symbol] 1%. The preceding ranges are intended to be made clear by context, and no further limitation is implied. The ranges can be made clear by context, and no further limitation will be shown. Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers. The foregoing applies regardless of whether in particular cases some or all of these embodiments are explicitly disclosed.

A carbon range (e.g., C₁-C₁₀), is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed therein, such as C₂-C₉, C₃-C₈, C₁-C₅, etc.

The term “derivative” and “derivatives” can be used to indicate compounds that are similar to a parent compound but different from it with respect to functional groups, atoms, etc. They may obey a specific chemical structure. A series of chemical structures of derivatives can be generated by the replacement of one or more functional groups, the introduction, or the removal of one or more substituents of the hydrogen atoms of the parent compound.

The term “halogen” refers to fluorine (F), chlorine (Cl), bromine (Br), iodine (I), and astatine (At).

The term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, a cyclic C₁-C₃₀ alkyl, a linear or branched C₁-C₃₀ alkyl, a linear or cyclic C₁-C₃₀ alkyl, a branched or cyclic C₁-C₃₀ alkyl, or a linear, branched, or cyclic C₁-C₃₀ alkyl.

The term “heteroalkyl” refers to alkyl groups where one or more carbon atoms are replaced with a heteroatom, such as, O, N, or S.

Heteroalkyl groups can be linear, branched, or cyclic (either monocyclic or polycyclic). Preferred heteroalkyl groups have one to 30 carbon atoms, i.e., C₁-C₃₀ heteroalkyl. In some forms, a C₁-C₃₀ heteroalkyl can be a linear C₁-C₃₀ heteroalkyl, a branched C₁-C₃₀ heteroalkyl, a cyclic C₁-C₃₀ heteroalkyl, a linear or branched C₁-C₃₀ heteroalkyl, a linear or cyclic C₁-C₃₀ heteroalkyl, a branched or cyclic C₁-C₃₀ heteroalkyl, or a linear, branched, or cyclic C₁-C₃₀ heteroalkyl.

The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic (either monocyclic or polycyclic). Preferred alkenyl groups have one to 30 carbon atoms, i.e., C₂-C₃₀ alkenyl. In some forms, a C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl.

The term “heteroalkenyl” refers to alkenyl groups in which one or more doubly bonded carbon atoms are replaced by a heteroatom. Heteroalkenyl groups can be linear, branched, or cyclic (either monocyclic or polycyclic). Preferred heteroalkenyl groups have one to 30 carbon atoms, i.e., C₁-C₃₀ heteroalkenyl. In some forms, a C₁-C₃₀ alkenyl can be a linear C₁-C₃₀ heteroalkenyl, a branched C₁-C₃₀ heteroalkenyl, a cyclic C₁-C₃₀ heteroalkenyl, a linear or branched C₁-C₃₀ heteroalkenyl, a linear or cyclic C₁-C₃₀ heteroalkenyl, a branched or cyclic C₁-C₃₀ heteroalkenyl, or a linear, branched, or cyclic C₁-C₃₀ heteroalkenyl.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic (either monocyclic or polycyclic). Preferred alkynyl groups have one to 30 carbon atoms, i.e., C₂-C₃₀ alkynyl. In some forms, a C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl.

The term “heteroalkynyl” refers to alkynyl groups in which one or more triply bonded carbon atoms are replaced by a heteroatom. Heteroalkynyl groups can be linear, branched, or cyclic (either monocyclic or polycyclic). Preferred heteroalkynyl groups have one to 30 carbon atoms, i.e., C₂-C₃₀ heteroalkynyl. In some forms, a C₂-C₃₀ alkynyl can be a linear C₂-C₃₀ heteroalkynyl, a branched C₂-C₃₀ heteroalkynyl, a cyclic C₂-C₃₀ heteroalkynyl, a linear or branched C₂-C₃₀ heteroalkynyl, a linear or cyclic C₂-C₃₀ heteroalkynyl, a branched or cyclic C₂-C₃₀ heteroalkynyl, or a linear, branched, or cyclic C₂-C₃₀ heteroalkynyl.

The term “aryl” refers to univalent groups derived from arenes by removal of a hydrogen atom from ring atom. Arenes are monocyclic or polycyclic aromatic hydrocarbons. In polycyclic arenes, the rings can be attached together in a pendant manner or can be fused. Preferred arenes have 6 to 50 carbon atoms, i.e., C₆-C₅₀ arenes. In some forms, a C₆-C₅₀ alkynyl can be a linear C₆-C₅₀ arenes, a branched C₆-C₅₀ arenes, a cyclic C₆-C₅₀ arenes, a linear or branched C₆-C₅₀ arenes, a linear or cyclic C₆-C₅₀ arenes, a branched or cyclic C₆-C₅₀ arenes, or a linear, branched, or cyclic C₆-C₅₀ arenes. Accordingly, in polycyclic aryl groups, the rings can be attached together in a pendant manner or can be fused. Preferred aryl groups have 6 to 50 carbon atoms, i.e., C₆-C₅₀ aryl. In some forms, a C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl.

The term “heteroaryl” refers to univalent groups derived from heteroarenes by removal of a hydrogen atom from a ring atom. Heteroarenes are heterocyclic compounds derived from arenes by replacement of one or more methine (—HC═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). Heteroarenes can be monocyclic or polycyclic. In polycyclic heteroarenes, the rings can be attached together in a pendant manner or can be fused. Preferred heteroarenes have 3 to 50 carbon atoms, i.e., C₃-C₅₀ heteroarenes. In some forms, a C₃-C₅₀ heteroarenes can be a branched C₃-C₅₀ heteroarenes, a monocyclic C₃-C₅₀ heteroarenes, a polycyclic C₃-C₅₀ heteroarenes, a branched polycyclic C₃-C₅₀ heteroarenes, a fused polycyclic C₃-C₅₀ heteroarenes, or a branched fused polycyclic C₃-C₅₀ heteroarenes.

Accordingly, in polycyclic heteroaryl groups, the rings can be attached together in a pendant manner or can be fused. Preferred heteroaryl groups have 3 to 50 carbon atoms, i.e., C₃-C₅₀ heteroaryl. In some forms, a C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl.

The term “arylene” refers to divalent groups derived from arenes by removal of a hydrogen atom from two ring carbon atoms. In polycyclic arylene groups, the rings can be attached together in a pendant manner or can be fused. Preferred arylene groups have 6 to 50 carbon atoms, i.e., C₆-C₅₀ arylene. In some forms, a C₆-C₅₀ arylene can be a branched C₆-C₅₀ arylene, a monocyclic C₆-C₅₀ arylene, a polycyclic C₆-C₅₀ arylene, a branched polycyclic C₆-C₅₀ arylene, a fused polycyclic C₆-C₅₀ arylene, or a branched fused polycyclic C₆-C₅₀ arylene.

The term “heteroarylene” refers to divalent groups derived from heteroarenes by removal of a hydrogen atom from two ring atoms. In polycyclic heteroarylene groups, the rings can be attached together in a pendant manner or can be fused. Preferred heteroarylene groups have 3 to 50 carbon atoms, i.e., C₃-C₅₀ heteroalkenyl. In some forms, a C₃-C₅₀ heteroarylene can be a branched C₃-C₅₀ heteroarylene, a monocyclic C₃-C₅₀ heteroarylene, a polycyclic C₃-C₅₀ heteroarylene, a branched polycyclic C₃-C₅₀ heteroarylene, a fused polycyclic C₃-C₅₀ heteroarylene, or a branched fused polycyclic C₃-C₅₀ heteroarylene.

The term “aminooxy” refers to —O—NH₂, wherein the hydrogen atoms can be substituted with substituents.

The term “hydroxyamino” refers to —NH—OH, wherein the hydrogen atoms can be substituted with substituents.

The term “hydroxamate” refers to —(C═O)—NH—OH, wherein the hydrogen atoms can be substituted with substituents.

The term “conjugated system” refers to a molecular entity whose structure can be represented as a system of alternating single and multiple bonds, e.g., —CH₂═CH— —CH═CH₂, —CH₂═CH—C≡N—. In such systems, conjugation is the interaction of one p-orbital with another across an intervening σ-bond in such structures. Conjugated systems can be or contain arene and/or heteroarene moieties.

The term “substituted,” as used herein, means that the chemical group or moiety contains one or more substituents replacing the hydrogen atoms in the chemical group or moiety. The substituents include, but not limited to: a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —NCS, —CF₃, —CH₂CF₃, —CH₂Cl, —COF, —COBr, —COOH, —S₃H, —CH₂SO₂CH₃, —PO₃H₂, —OPO₃H₂, —P(═O)(OR^{G1′})(OR^{G2′}), —OP(═O)(OR^{G1′})(OR^{G2′}), BR^{G1′}(OR^{G2′}), —B(OR^{G1′})(OR^{G2′}), or -G′R^{G1′} in which -G′ is —O—, —S—, —NR^{G2′}—, —C(═O)O—, —C(═O)—, —S(═O)—, —SO₂—, —C(═O)O—, —C(═O)NR^{G2′}—, —NR^{G2′}C(O)—, —NR^{G2′}C(═O)NR^{G3′}—, —C(═S)—, —C(═S)S—, —SC(═S)—, —SC(═S)S—, —C(═NR^{G2′})—, —C(═NR^{G2′})O—, —C(═NR^{G2′})R^{G3′}—, —OC(═NR^{G2′})—, —NR^{G2′}C(═NR^{G3′})—, —NR^{G2′}SO₂—, —C(═NR^{G2′})NR^{G′})—, —OC(═NR^{G2′})—, —NR^{G2′}SO₂NR^{G3′}—, —NR^{G2′}C(═S)—, —SC(═S)NR^{G2′}—, —NR^{G2′}C(═S)S—, —NR^{G2′}C(═S)NR^{G3′}—, —SC(═NR^{G2′})—C(═S)NR^{G2′}—, —OC(═S)NR^{G2′}—, NR^{G2′}C(═S)O—, —SC(═O)NR^{G2′}—, —NR^{G2′}C(O)S—, —C(O)S—, —SC(O)—, —SC(O)S—, —C(═S)O—, —OC(═S)—, —OC(═S)O—, —SO₂NR^{G2′}—, —BR^{G2′}—, or —PR^{G2′}—.

Wherein each occurrence of R^{G1′}, R^{G2′}, and R^{G3′} is independently a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

The terms “d⁸ or d¹⁰ metal complex” and “d⁸ or d¹⁰ metal complexes” employed in this application can be any complex containing at least one metal center with a d⁸ or d¹⁰ electronic configuration. The term “d⁸ or d¹⁰ metal complex aggregate” refers to local concentration enrichment of the d⁸ or d¹⁰ metal complex in the vicinity of an analyte. The analyte can be sialic acids or polysialic acids, glycans, or cancer cells. The local concentration enrichment can be caused by noncovalent metal-metal interactions between molecules of the d⁸ or d¹⁰ metal complex. Noncovalent interactions, such as π-π stacking interactions, electrostatic interactions, hydrogen bonding interactions, and hydrophobic interactions, and combinations thereof can contribute to the binding between the analyte and the d⁸ or d¹⁰ metal complex and between different molecules of the d⁸ or d¹⁰ metal complex. In some forms, the d⁸ or d¹⁰ metal complex aggregate can be formed via aggregation and supramolecular self-assembly of the d⁸ or d¹⁰ metal complex after binding to the analyte. The metal center can be selected from the group consisting of Pt(II), Pd(II), Ni(II), Ir(I), Rh(I), Au(III), Ag(III), Cu(III), Ni(O), Pd(O), Pt(O), Cu(I), Ag(I), Au(I), Zn(II), Cd(II), and Hg(II).

The terms “ligand” and “ligands” refer to ions or molecules that bind to a metal center via one or more donor atoms, thereby forming a metal complex. The nature of the metal-ligand bonding can range from covalent to ionic. The metal-ligand bond order can range from one to three. The bonding with the metal center generally involves formal donation of one or more electron pairs from the donor atoms. The donor atoms can be carbon (C), nitrogen (N), oxygen (O), phosphorus (P), sulfur (S), arsenic (As), and selenium (Se).

The term “coordination number” refers to the total number of donor atom.

The term “sialic acid” employed in this application can be a member from a class of alpha-keto acid sugars sharing a nine-carbon backbone.

The term “polysialic acids” employed in this application can be a polymer of linearly repeating monomer units of α-2,8 and α-2,9 glycosidic linked sialic acid residues.

The term “luminescence” refers to emission of light by a substance not resulting from heat. It can be caused by chemical reactions, electrical energy, subatomic motions or stress on a crystal, which all are ultimately caused by spontaneous emission. It can refer to chemiluminescence, i.e., the emission of light as a result of a chemical reaction. It can also refer to photoluminescence, i.e., the emission of light as a result of absorption of photons. The photoluminescence includes fluorescence, and phosphorescence, thermally activated delayed fluorescence (TADF), thermally stimulated delayed phosphorescence (TSDP), upconversion luminescence and other forms of luminescence on light excitation.

The terms “carrier” and “carriers” refer to all components present in a formulation or composition other than the active ingredient or ingredients.

They can include but are not limited to, diluents, binders, lubricants, disintegrators, fillers, plasticizers, pigments, colorants, stabilizing agents, and glidants.

As used herein, “subject” includes, but is not limited to, human or non-human mammals. The term does not denote a particular age or sex.

Thus, adult and newborn subjects, as well as fetuses, whether male or female, or bisexual, are intended to be covered.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

It is also to be understood that when a certain geometry is specified for the compounds, e.g., linear, trigonal planar, and square planar, the specified geometry can be ascertained from the atom(s) in a ligand(s) involved in bonding with the central metal atom.

### II. Compositions

Disclosed are compounds that can be utilized to detect and/or visualize an analyte or test the efficacy of inhibitors. In some forms, the analyte can be sialic acids, polysialic acids, glycans, and cancer cells. The disclosed compound can be used to sense the analytes and differentiate cancer cell from normal cells. The compound can be used for detection or diagnosis of signs of cancer. The compound can also be utilized to screen inhibitors that can remove glycans, such as sialic acids, polysialic acids, etc., on cellular surfaces for the therapy. Some examples will be provided in this application. In some forms, the compounds are metal complexes with d⁸ or d¹⁰ electronic configuration composed of at least one metal center and at least one coordinating ligand:
(a) a metal center with a coordination number of 2, 3, or 4, selected from Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), and Hg(II); and/or
one or more ligands with donor atoms selected from the group containing carbon (C), nitrogen (N), oxygen (O), phosphorus (P), sulfur (S), arsenic (As), and selenium (Se).

### A. Metal Complexes or Compounds

The d⁸ or d¹⁰ metal complexes exhibit square-planar, trigonal-planar, partially planar, or linear structures and the geometry facilitates stacking of the metal complexes with each other, thereby generating the aggregates. The self-assembled structures and/or aggregates can form highly ordered linear structures, oligomers, or hierarchical superstructures. Noncovalent interactions induced by the introduction of specific functional groups endow metal complexes with the ability to bind to the analytes of interest. Non-limiting examples of functional groups that drive this specificity are listed as follows.

They can be selected from the positively charged functional groups:

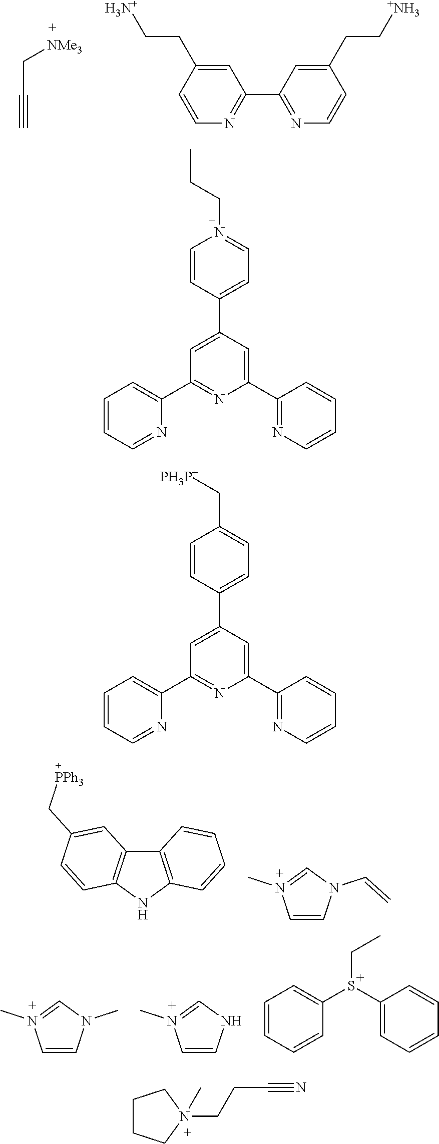

They can be selected from amino acids and/or their derivatives, or combination of amino acids and/or their derivatives:

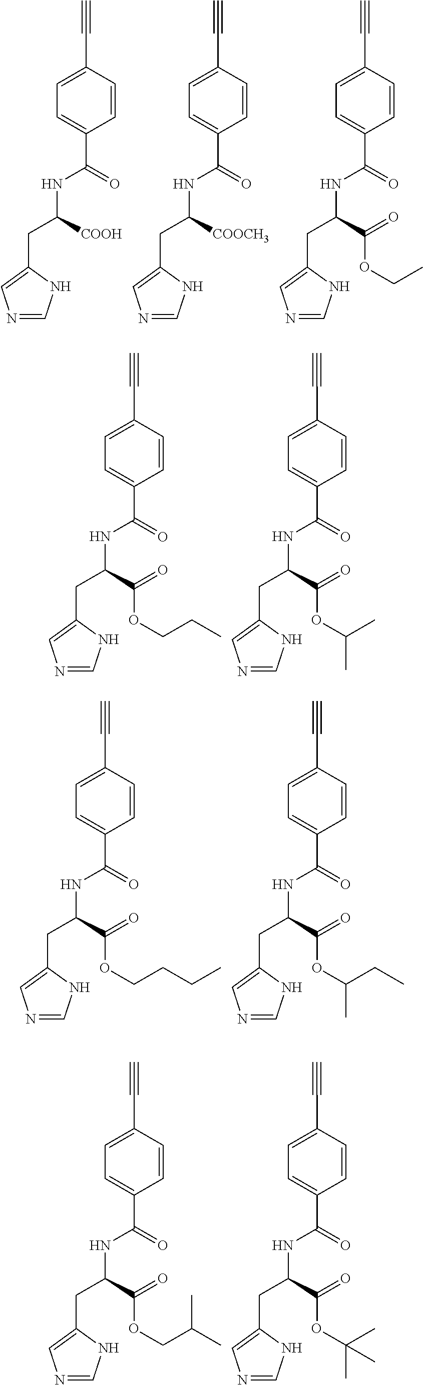

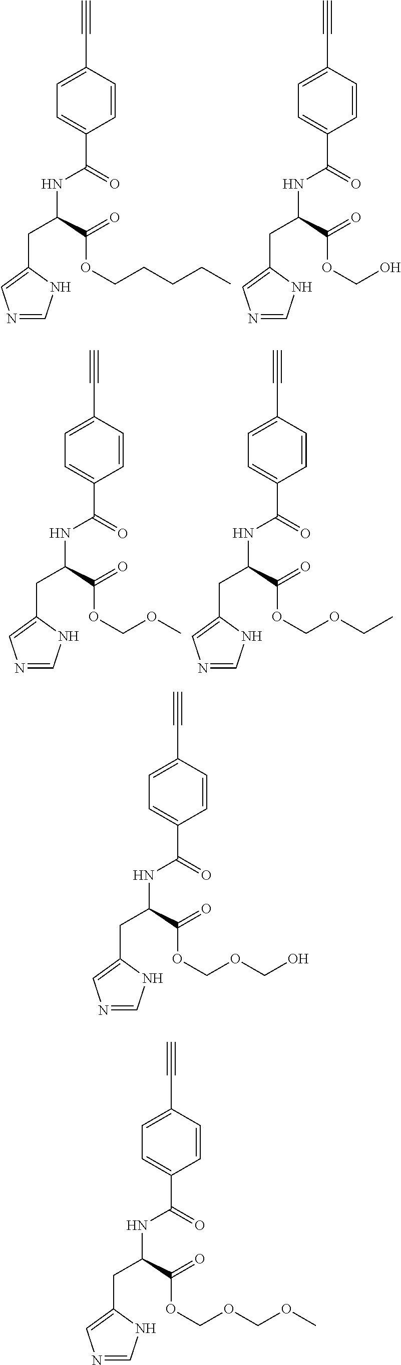

Noncovalent interactions may include electrostatic interactions, hydrogen bonding interactions, hydrophobic interaction, and combinations thereof. The specific binding interactions between the metal complex and the analyte facilitate the neighboring complex molecules to come into proximity, leading to the supramolecular self-assembly of the metal complexes. The electrostatically and/or noncovalent interaction bound metal complex-analyte adducts or ensembles are further stabilized by the noncovalent metal-metal interactions and/or π-π stacking interactions arising from the self-assembly and/or aggregation of the metal complexes and are not easily disrupted by non-specific interactions.

In some forms, the analytes which can induce the assembly/disassembly of metal complexes can be sialic acids or polysialic acids, or glycans, or cancer cells. Sialic acids share a nine-carbon backbone and are usually located at the outermost end of the glycan chains of all cell types. The analyte also includes the dimer, trimer, or polymer containing sialic acids.

In some forms, the metal complexes can bind to some kinds of monosaccharides, including but not limited to monosaccharides, disaccharides, and polysaccharides. Monosaccharides have the formula of CₙH₂ₙOₙ. Examples of monosaccharides can be but not limited to glucose, galactose, fructose, etc. Examples of disaccharides can be but not limited to sucrose, lactose, etc. Examples of polysaccharides can be but not limited to cellulose, starch, etc. The specific binding interactions between compounds and the analytes facilitate self-assembly and aggregation of the metal complexes. The self-assembly and/or aggregation of the metal complexes induces changes in the photophysical properties of metal complexes. Photophysical properties can be but not limited to absorbance, luminescence, circular dichroism, circularly polarized luminescence, or combinations thereof.

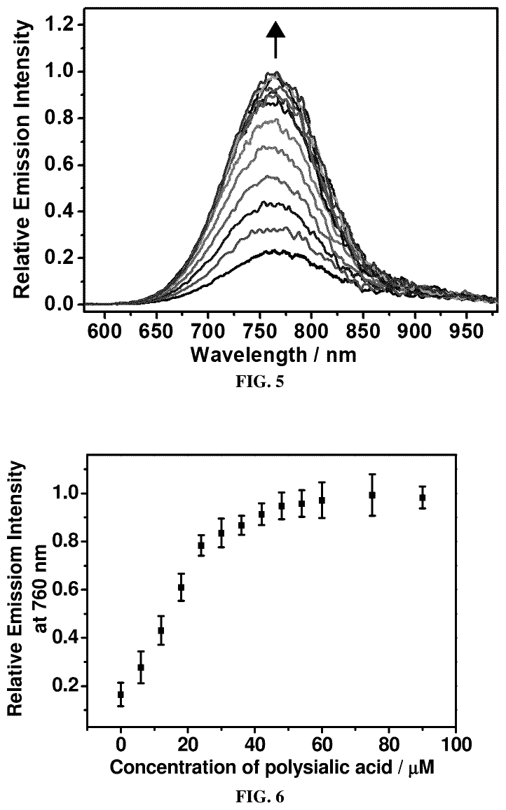

In some forms, the variation of luminescence intensity, upon addition of an analyte, can be observed as shown in FIG. 5. Preferably, an apparent difference of emission wavelength or energy in the absence and presence of an analyte can be observed. Noncovalent interactions, including electrostatic interactions, hydrogen bonding interactions, hydrophobic interaction, and combinations thereof, facilitate the interaction between the metal complex and the analyte, leading to the supramolecular self-assembly of the metal complex. The variation in photophysical properties can be or include the change in a luminescence band in the red to near-infrared (NIR I and NIR II) emission, for example, in the range from about 600 nm to about 1700 nm. The changes include a blue shift or a red shift of the emission energy or wavelength, or emission intensity, resulting from the variation of supramolecular self-assembly of the metal complexes. In some forms, a large Stokes shifts can also be observed. In some forms, the Stokes shift is larger than 100 nm, larger than 150 nm, larger than 200 nm, larger than 250 nm, larger than 300 nm, larger than 350 nm, or larger than 400 nm. More preferably, the Stokes shift is larger than 400 nm.

In some forms, the specificity of the metal complexes toward an analyte is due to one or more specific noncovalent interactions. The design strategies can be used to introduce more than one noncovalent interaction for better specificity. Then the planar, trigonal planar, partially planar, or linear geometry endows the metal complex with the tendency to form a highly ordered structure. The binding interaction between the metal complex and the analyte would make the metal complex molecules come into proximity, favoring the formation of self-assembly and/or aggregation. The metal complex can be designed to bind an analyte of interest by selecting the metal center and or the coordination ligands, particularly the functional groups on the ligands of the metal complexes. The self-assembly of the metal complex can be known, greatly prompting the design of these detection methods.

In some forms, d⁸ or d¹⁰ metal complexes would be selected as a sensor to bind with an analyte. The introduction of one or more specific functional groups to the ligands gives rise to a high tendency of the metal complex to form strong binding interactions with the analyte, increasing the specificity. The combination of different metal centers and ligands will generate useful sensors to bind with an analyte.

In some forms, the repeatedly high-order structure of an analyte facilitates the supramolecular self-assembly of the metal complex after binding. In some forms, electrostatic interactions between a charged metal complex and an analyte with electrostatically opposite charge can facilitate the binding between them. In some forms, the hydrogen bonding between a metal complex and an analyte can benefit the strong binding interaction. In some forms, other noncovalent interactions, including but not limited to, hydrophobic interaction, and π-π stacking interaction can assist the binding interaction between the metal complex and the analyte. The electrostatically and/or noncovalently bound metal complex-analyte adducts or ensembles are further stabilized by the noncovalent metal-metal interactions and/or π-π stacking interactions arising from the self-assembly and/or aggregation of the metal complexes and are not easily disrupted by non-specific interactions.

### B. Ligands of the Metal Complexes or Compounds

There are four types of ligands that can be included in these complexes: monodentate, bidentate, tridentate, and tetradentate ligands. The bonding between the ligands to the metal centers in the metal complexes generally involves formal donation of one or more electron pairs from the donor atoms of the ligands. The donor atoms can be carbon (C), nitrogen (N), oxygen (O), phosphorus (P), sulfur (S), arsenic (As), and selenium (Se).

In some forms, the ligand has a chemical structure shown by a generic Formula IV:

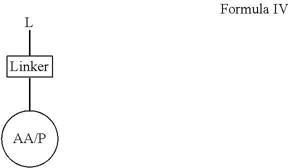

### Wherein:

L stands for chemical moieties containing one or more donor atoms, preferably one donor atom, for coordination to the metal center of the metal complexes. Exemplary chemical moieties include, but are not limited to, five-membered arenes and their derivatives, such as furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; six-membered arenes and their derivatives, such as benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene; Exemplary ligands also include, but are not limited to, halide ions, SCN⁻ (donor atom: S), O—NO₂⁻ (donor atom: O), N₃⁻, O₂⁻, S₂⁻, H₂O, O—NO⁻ (donor atom: O), NCS⁻ (donor atom: N), NH₃, NO₂⁻ (donor atom: N), N≡C⁻ (donor atom: N), C≡N⁻ (donor atom: C), CO (donor atom: C), R—C≡C⁻, RO⁻—, RS⁻—, RSe⁻—, N═N═N—R, N≡C—R (donor atom: N), C≡N—R (donor atom: C), NR¹R²R³, PR¹R²R³, and AsR¹R²R³.

In some forms of these ligands, R, R¹, R², and R³ are independently: a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group; a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group; an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group; an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof, or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,

In some forms, R, R¹, R², R³, and their organic substituents are optionally and independently substituted with one or more groups, wherein each such group is independently:
a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —NCS, —CF₃, —CH₂CF₃, —CH₂Cl, —COF, —COBr, —COOH, —S₃H, —CH₂SO₂CH₃, —PO₃H₂, —OPO₃H₂, —P(═O)(OR^{G1′})(OR^{G2′}), —OP(═O)(OR^{G1′})(OR^{G2′}), —BR^{G1′}(OR^{G2′}), B(OR^{G1′})(OR^{G2′}), or -G′R^{G1′} in which -G′ is —O—, —S—, —NR^{G2′}—, —C(═O)O—, —C(═O)—, —S═O—, —SO₂—, —C(═O)O—, —C(═O)NR^{G2′}, —NR^{G2′}C(O)—, NR^{G2′}C(═O)NR^{G3′}—, —C(═S)—, —C(═S)—, —SC(═S)—, —SC(═S)S—, —C(═NR^{G2′})—, —C(═NR^{G2′})O—, —C(═NR^{G2′})R^{G3′}—, —OC(═NR^{G2′})—, NR^{G2′}C(═NR^{G3′})—, —NR^{G2′}SO₂—, —C(═NR^{G2′})NR^{G′})—, —OC(═NR^{G2′})—, —NR^{G2′}SO₂NR^{G3′}—, —NR^{G2′}C(═S)—, —SC(═S)NR^{G2′}—, —NR^{G2′}C(═S)S—, —NR^{G2′}C(═S)NR^{G3′}—, —SC(═NR^{G2′})—, —C(═S)NR^{G2′}—, —OC(═S)NR^{G2′}—, NR^{G2′}C(═S)O—, —SC(═O)NR^{G2′}—, —NR^{G2′}C(═O)S, —C(═O)S—, —SC(═O)—, —SC(═O)S—, —C(═S)O—, —OC(═S)—, —OC(═S)O—, —SO₂NR^{G2′}—, —BR^{G2′}—, or —PR^{G2′}—.

Wherein each occurrence of R^{G1′}, R^{G2′}, and R^{G3′} is independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

Linker stands for structures that facilitate the optional covalent linking moieties between ligands and amino acids/positive charge-containing structures. Linker can be selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and combination thereof.

AA are selected from the following structures of amino acids, or their derivatives, or combination of amino acid and/or their derivatives.

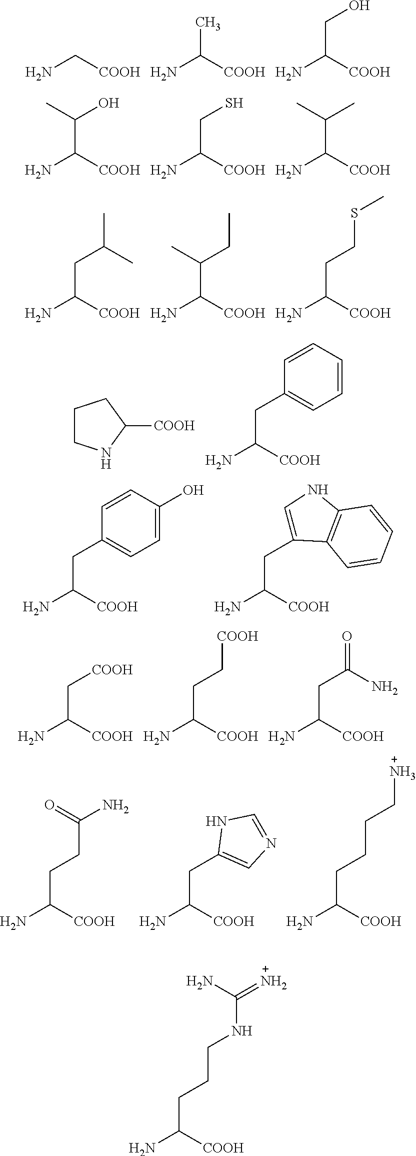

The exemplary structures of ligands in the Formula IV are shown below using one of these amino acids, histidine as examples. Histidine can be replaced with other amino acids or their derivatives, or combination of amino acids and/or their derivatives.

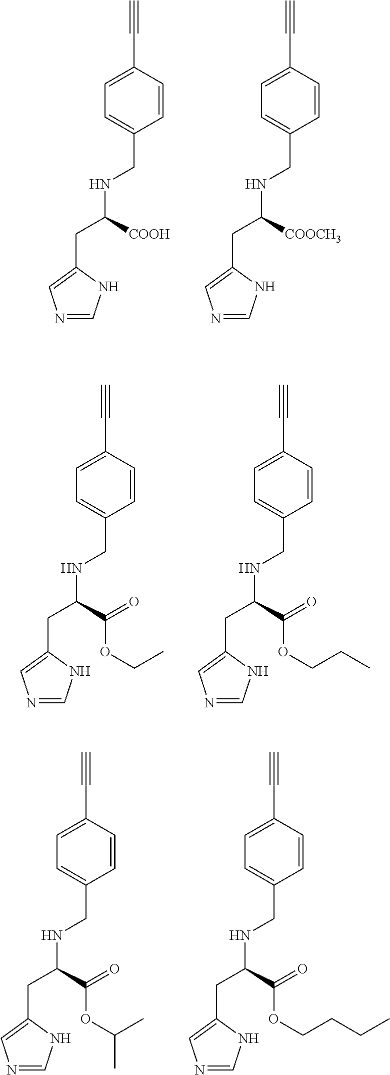

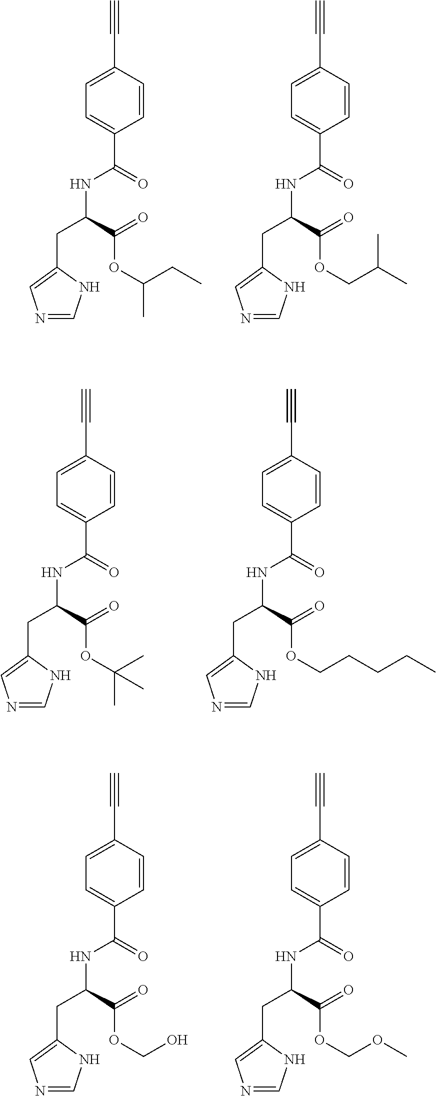

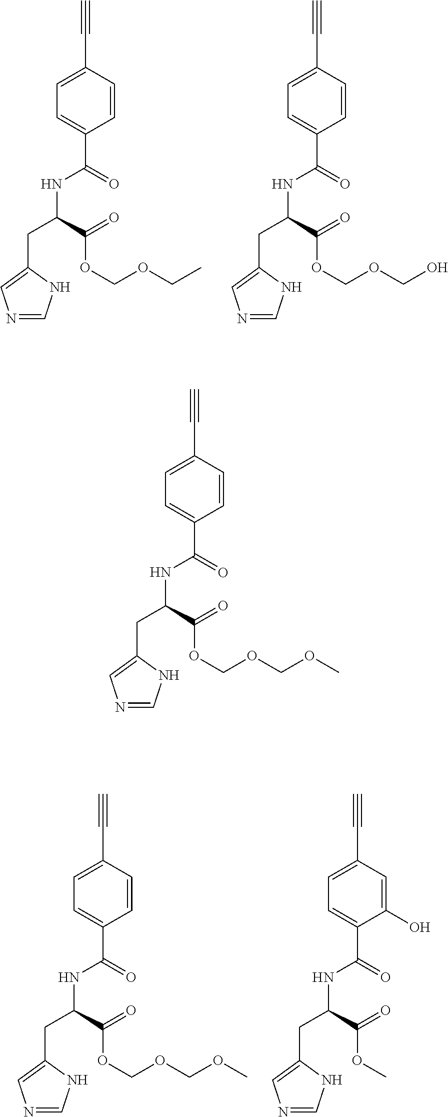

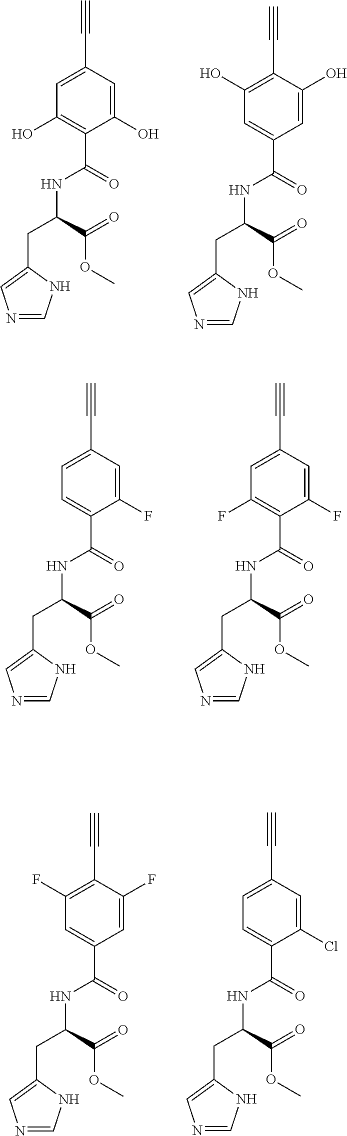 [SMILES: C#Cc1cc(O)c(C(=O)N[C@H](Cc2cnc[nH]2)C(=O)OC)c(O)c1 ; C#Cc1c(O)cc(C(=O)N[C@H](Cc2cnc[nH]2)C(=O)OC)cc1O ; C#Cc1ccc(C(=O)N[C@H](Cc2cnc[nH]2)C(=O)OC)c(F)c1 ; C#Cc1cc(F)c(C(=O)N[C@H](Cc2cnc[nH]2)C(=O)OC)c(F)c1 ; C#Cc1c(F)cc(C(=O)N[C@H](Cc2cnc[nH]2)C(=O)OC)cc1F ; C#Cc1ccc(C(=O)N[C@H](Cc2cnc[nH]2)C(=O)OC)c(Cl)c1]

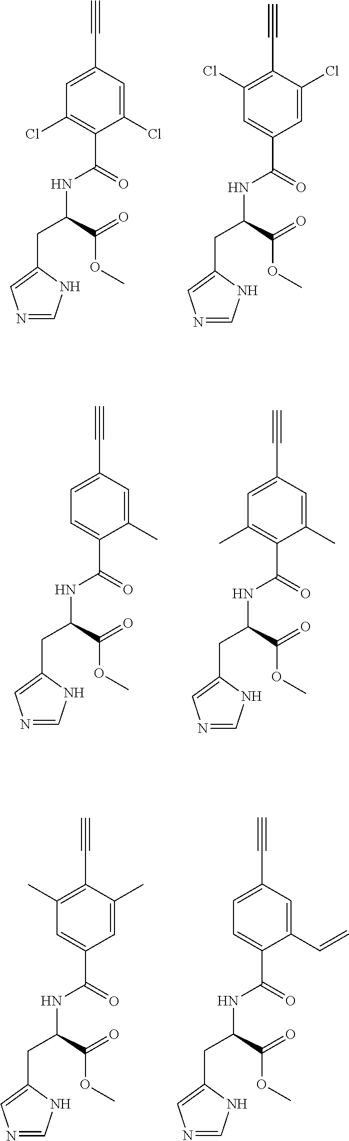 [SMILES: C#Cc1cc(Cl)c(C(=O)N[C@H](Cc2cnc[nH]2)C(=O)OC)c(Cl)c1 ; C#Cc1c(Cl)cc(C(=O)N[C@H](Cc2cnc[nH]2)C(=O)OC)cc1Cl ; C#Cc1ccc(C(=O)N[C@H](Cc2cnc[nH]2)C(=O)OC)c(C)c1 ; C#Cc1cc(C)c(C(=O)N[C@H](Cc2cnc[nH]2)C(=O)OC)c(C)c1 ; C#Cc1c(C)cc(C(=O)N[C@H](Cc2cnc[nH]2)C(=O)OC)cc1C ; C#Cc1ccc(C(=O)N[C@H](Cc2cnc[nH]2)C(=O)OC)c(C=C)c1]

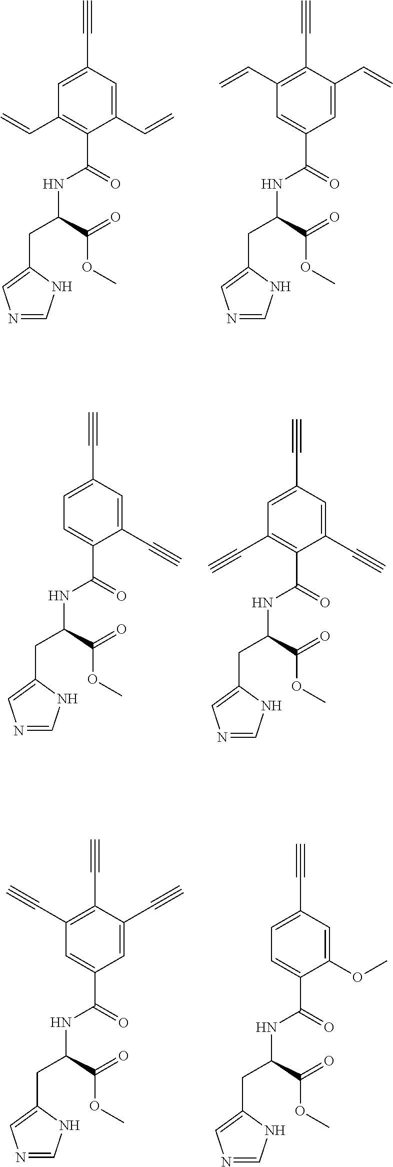

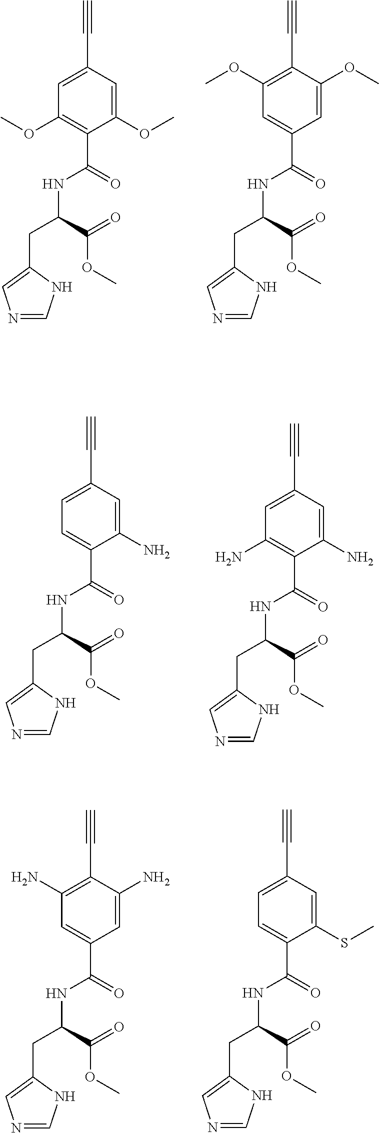

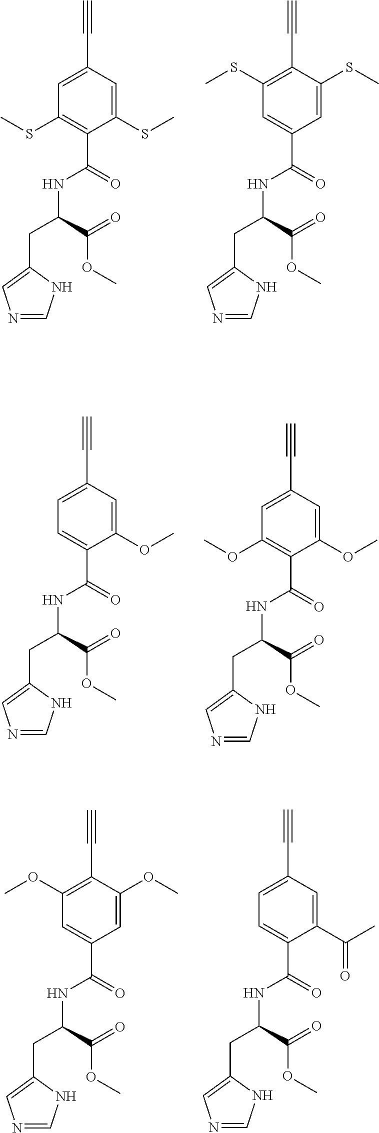

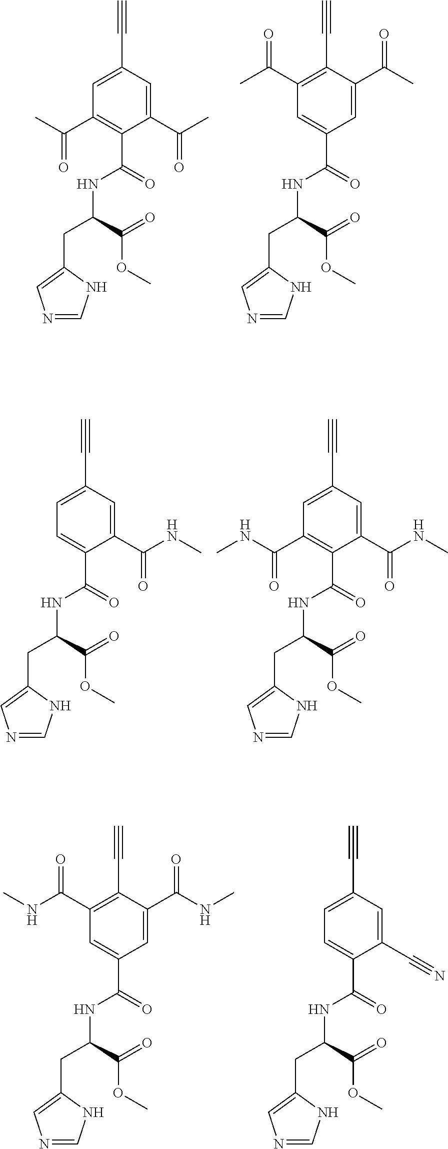

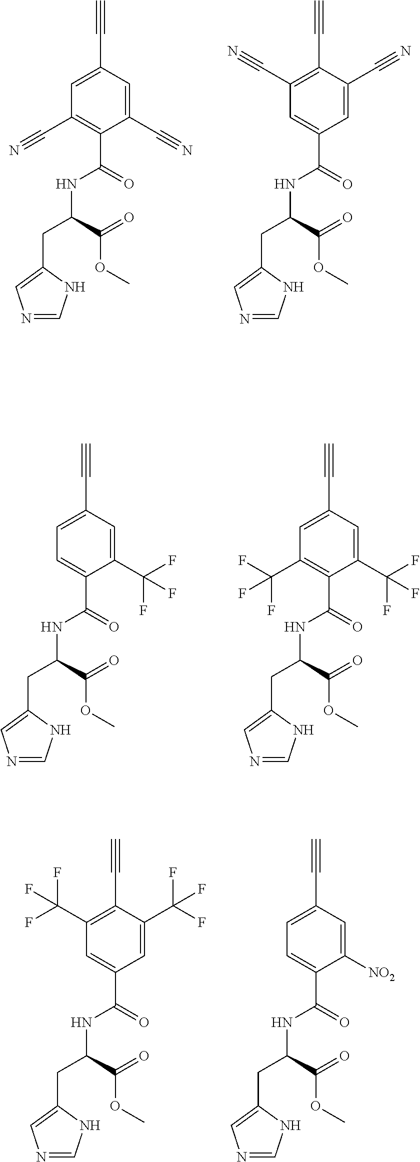 [SMILES: C#Cc1cc(C#N)c(C(=O)N[C@H](Cc2cnc[nH]2)C(=O)OC)c(C#N)c1 ; C#Cc1c(C#N)cc(C(=O)N[C@H](Cc2cnc[nH]2)C(=O)OC)cc1C#N ; C#Cc1ccc(C(=O)N[C@H](Cc2cnc[nH]2)C(=O)OC)c(C(F)(F)F)c1 ; C#Cc1cc(C(F)(F)F)c(C(=O)N[C@H](Cc2cnc[nH]2)C(=O)OC)c(C(F)(F)F)c1 ; C#Cc1c(C(F)(F)F)cc(C(=O)N[C@H](Cc2cnc[nH]2)C(=O)OC)cc1C(F)(F)F ; C#Cc1ccc(C(=O)N[C@H](Cc2cnc[nH]2)C(=O)OC)c([N+](=O)[O-])c1]

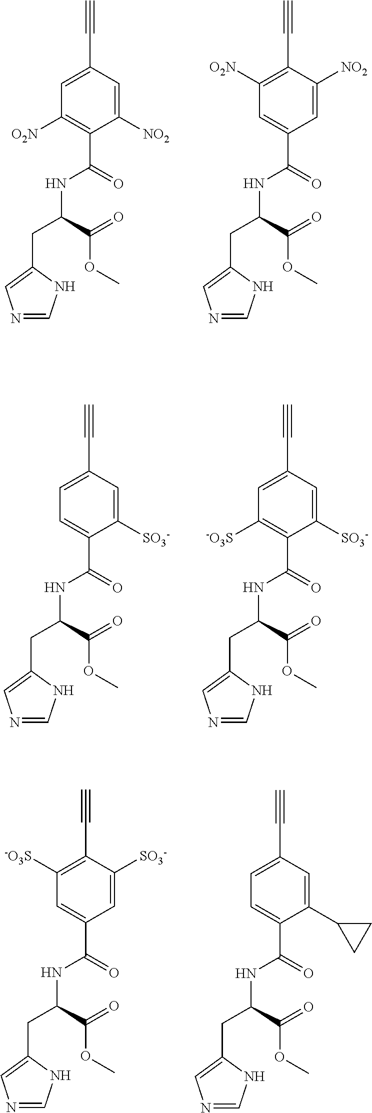

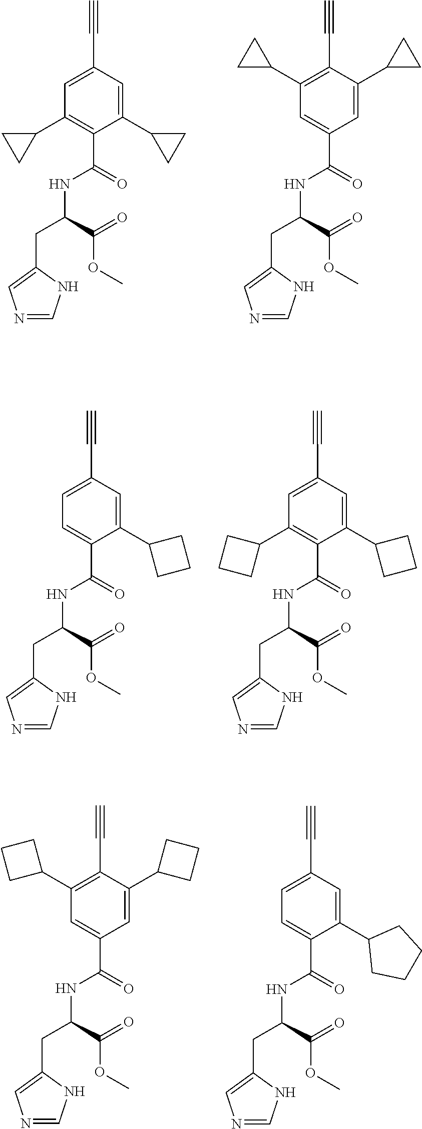

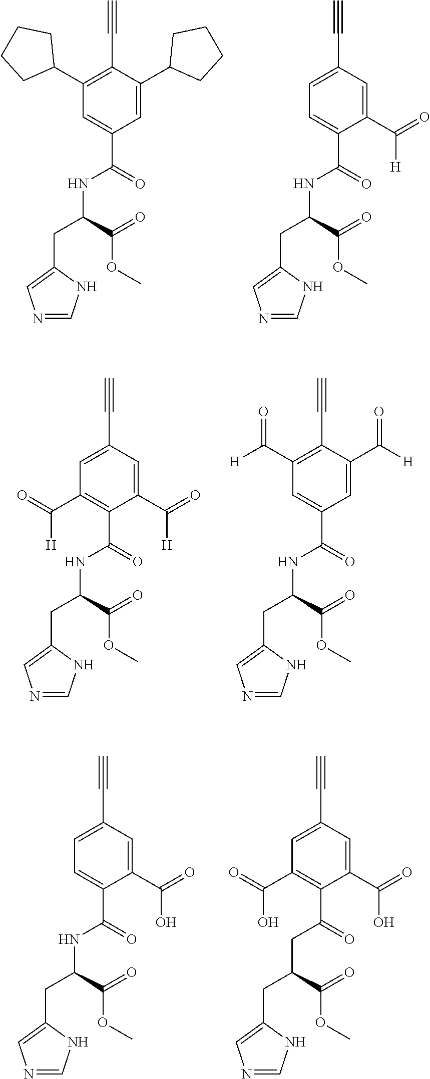

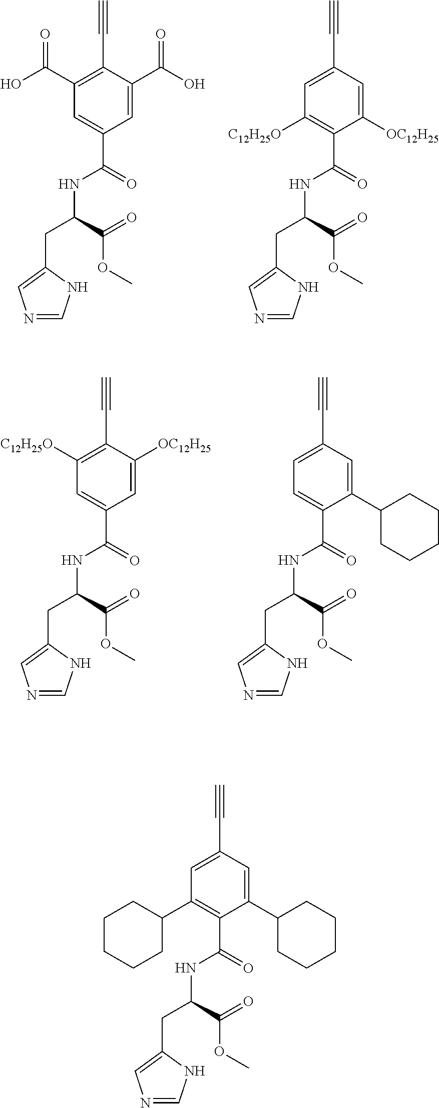

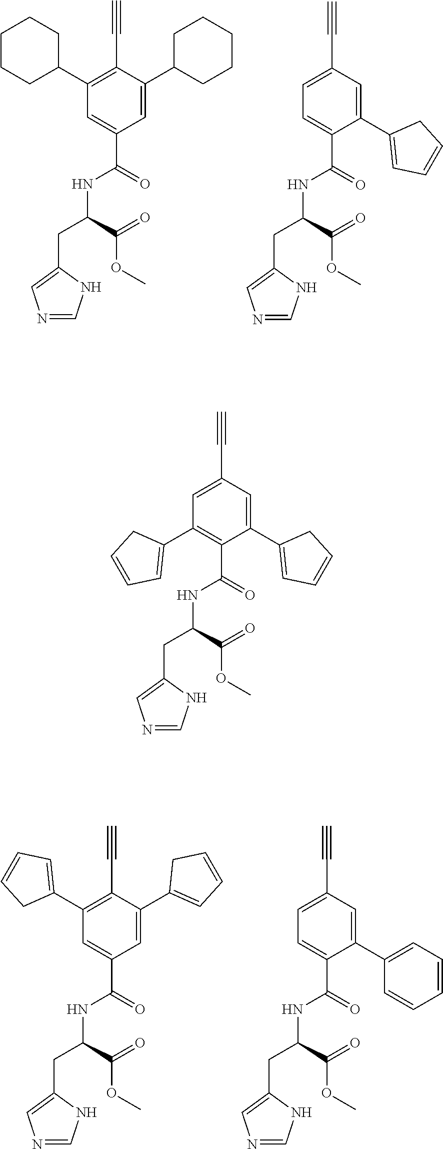 [SMILES: C#Cc1c(C2CCCCC2)cc(C(=O)N[C@H](Cc2cnc[nH]2)C(=O)OC)cc1C1CCCCC1 ; C#Cc1ccc(C(=O)N[C@H](Cc2cnc[nH]2)C(=O)OC)c(C2=CC=CC2)c1 ; C#Cc1cc(C2=CC=CC2)c(C(=O)N[C@H](Cc2cnc[nH]2)C(=O)OC)c(C2=CC=CC2)c1 ; C#Cc1c(C2=CC=CC2)cc(C(=O)N[C@H](Cc2cnc[nH]2)C(=O)OC)cc1C1=CC=CC1 ; C#Cc1ccc(C(=O)N[C@H](Cc2cnc[nH]2)C(=O)OC)c(-c2ccccc2)c1]

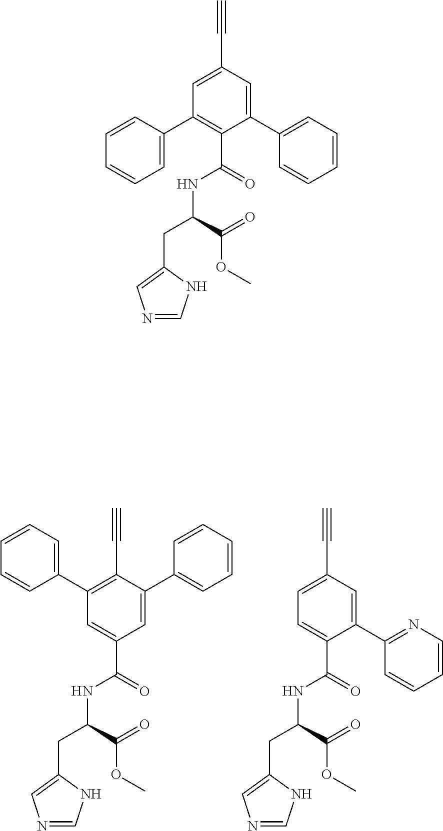 [SMILES: C#Cc1cc(-c2ccccc2)c(C(=O)N[C@H](Cc2cnc[nH]2)C(=O)OC)c(-c2ccccc2)c1 ; C#Cc1c(-c2ccccc2)cc(C(=O)N[C@H](Cc2cnc[nH]2)C(=O)OC)cc1-c1ccccc1 ; C#Cc1ccc(C(=O)N[C@H](Cc2cnc[nH]2)C(=O)OC)c(-c2ccccn2)c1]

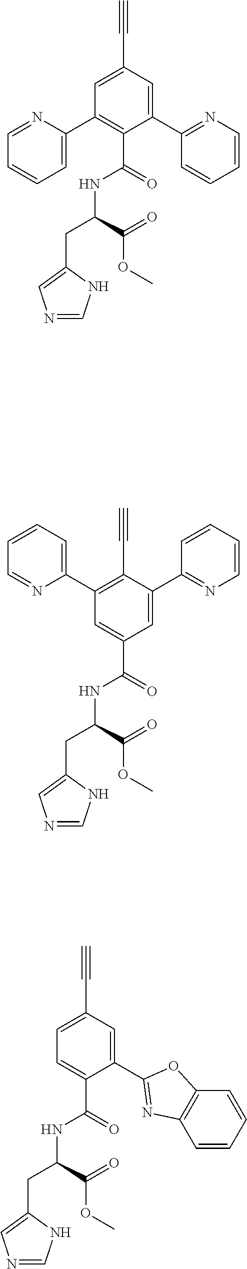 [SMILES: C#Cc1cc(-c2ccccn2)c(C(=O)N[C@H](Cc2cnc[nH]2)C(=O)OC)c(-c2ccccn2)c1 ; C#Cc1c(-c2ccccn2)cc(C(=O)N[C@H](Cc2cnc[nH]2)C(=O)OC)cc1-c1ccccn1 ; C#Cc1ccc(C(=O)N[C@H](Cc2cnc[nH]2)C(=O)OC)c(-c2nc3ccccc3o2)c1]

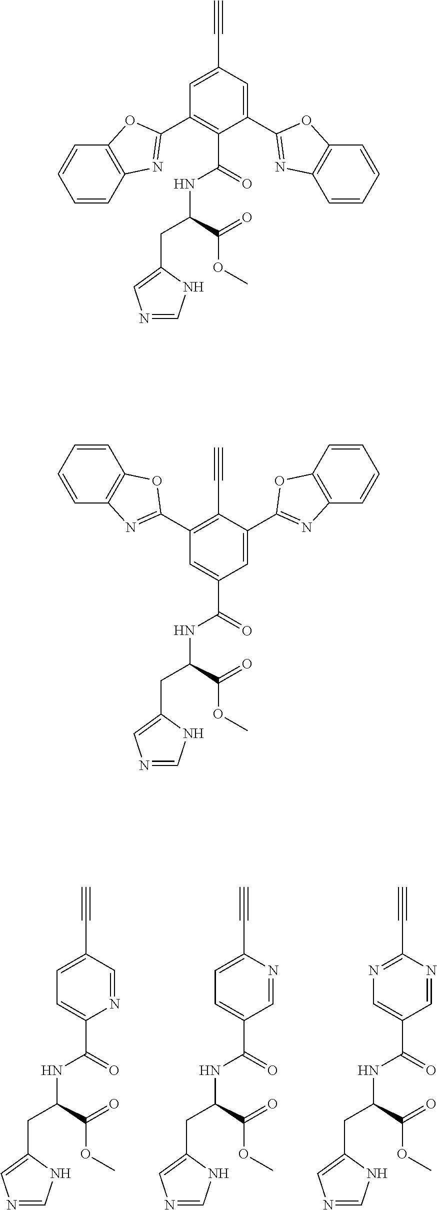

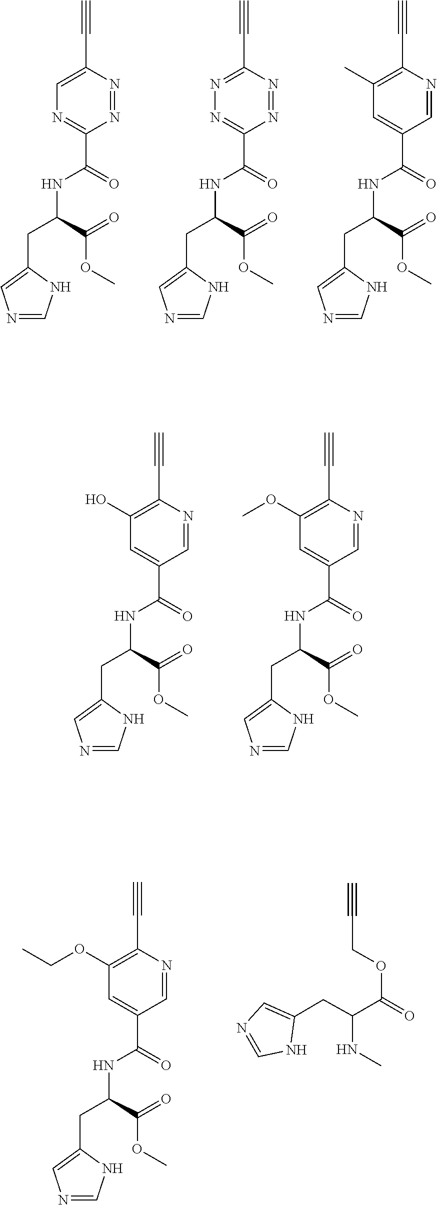

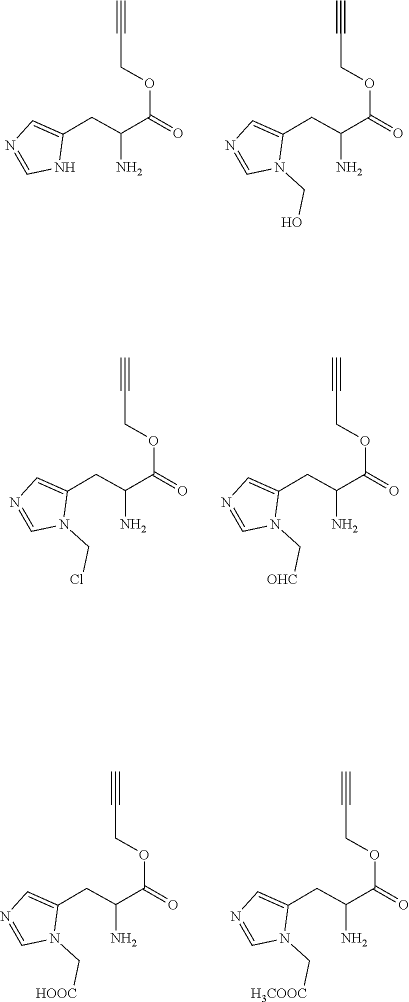

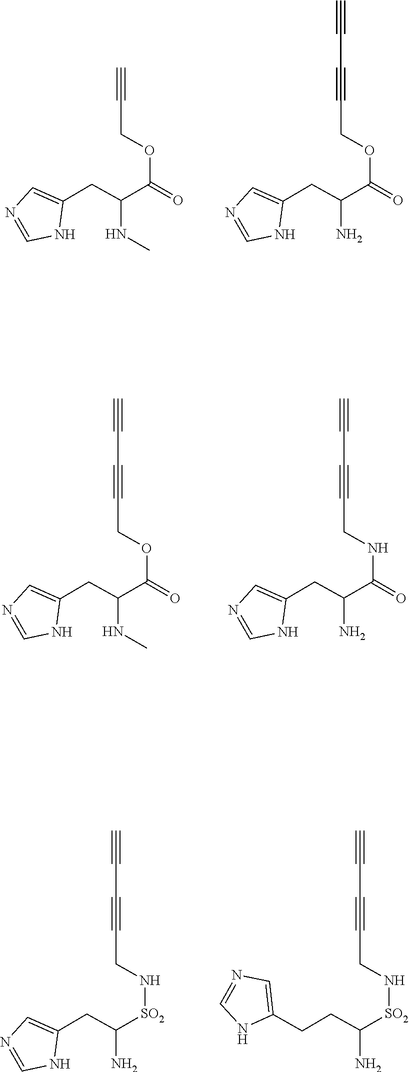 [SMILES: C#CCOC(=O)C(Cc1cnc[nH]1)NC ; C#CC#CCOC(=O)C(N)Cc1cnc[nH]1 ; C#CC#CCOC(=O)C(Cc1cnc[nH]1)NC ; C#CC#CCNC(=O)C(N)Cc1cnc[nH]1 ; C#CC#CCNS(=O)(=O)C(N)Cc1cnc[nH]1 ; C#CC#CCNS(=O)(=O)C(N)CCc1cnc[nH]1]

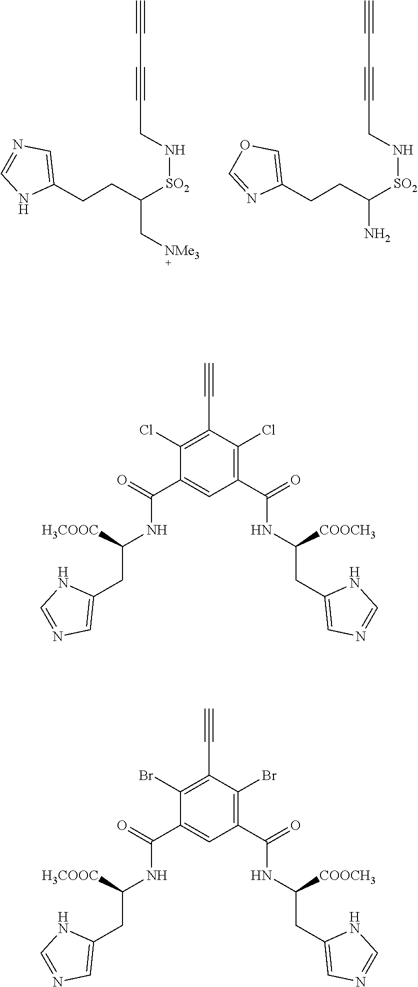

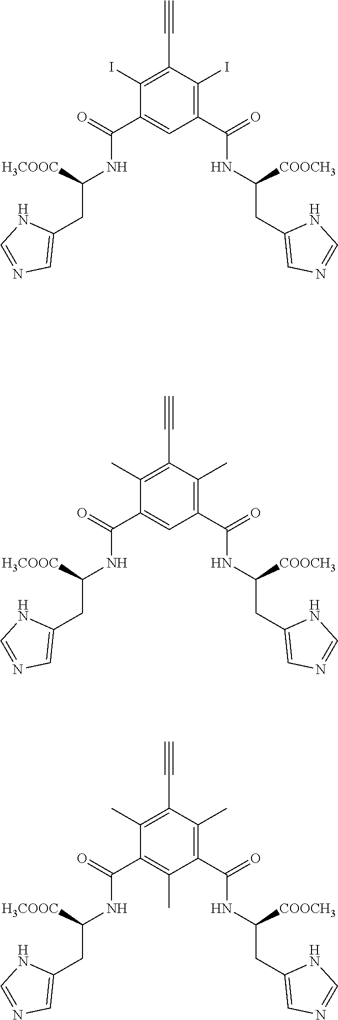

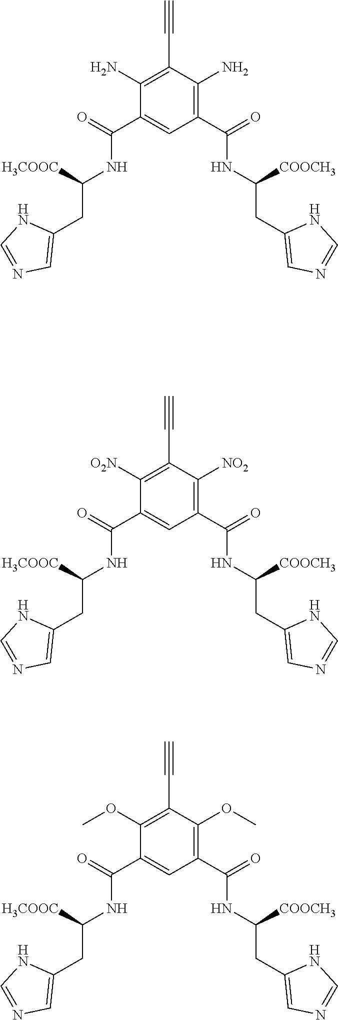

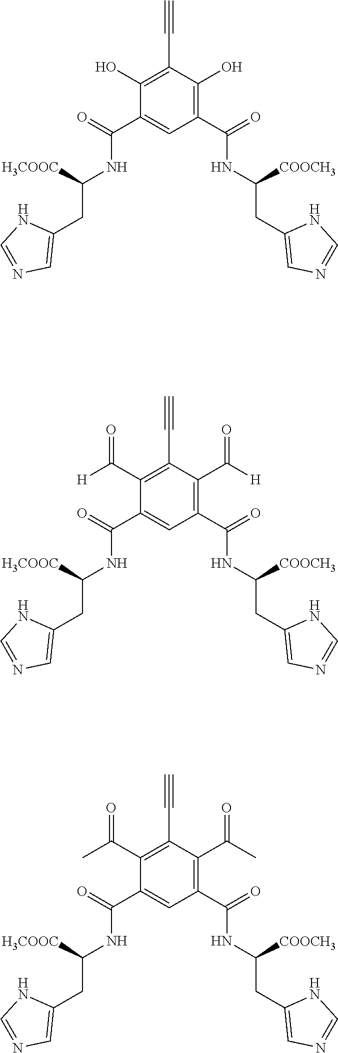

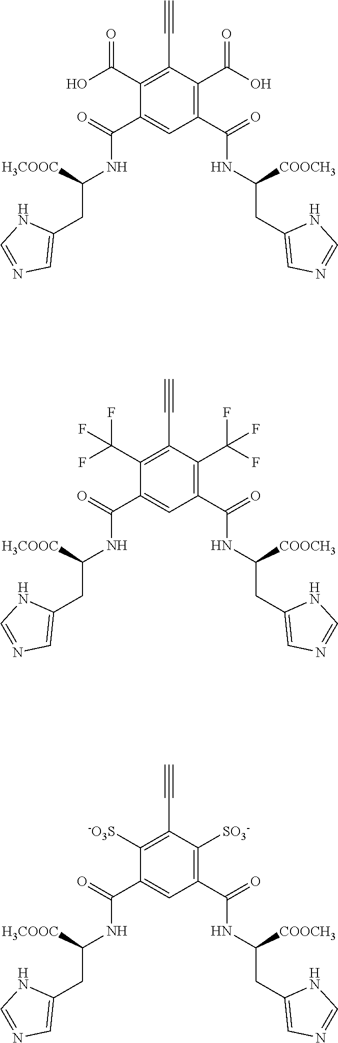

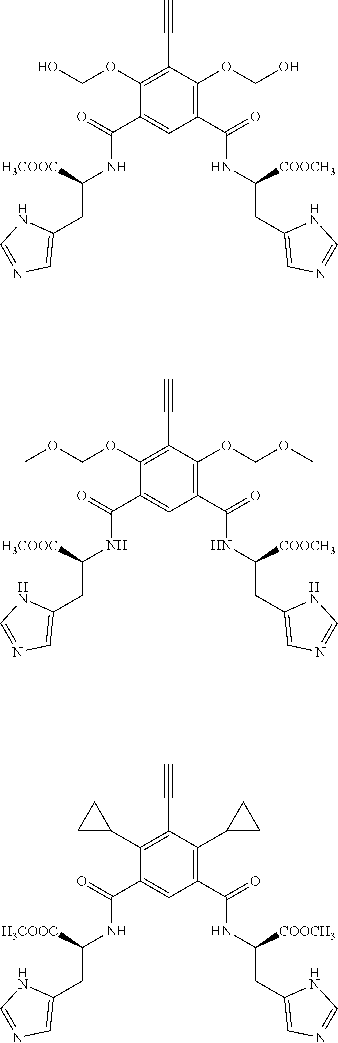

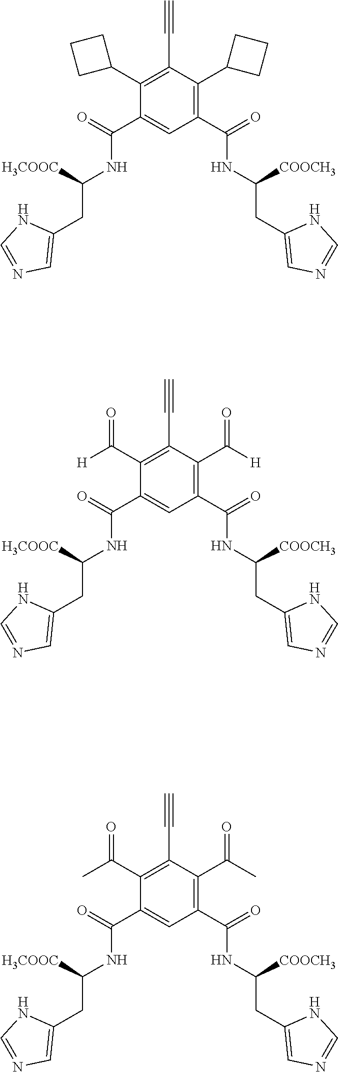

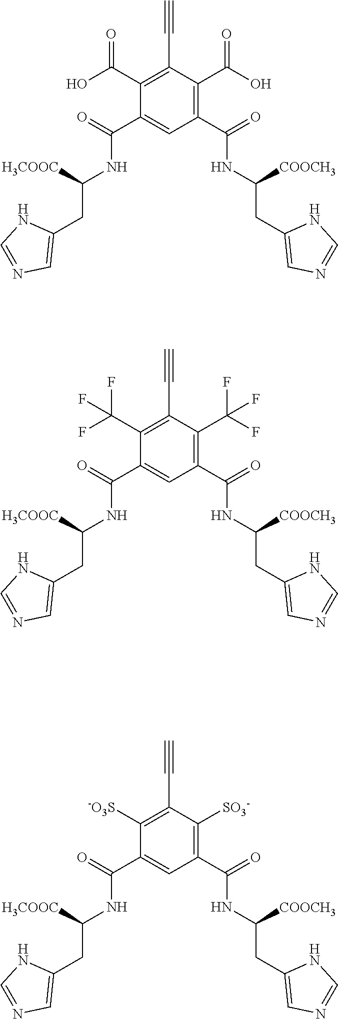

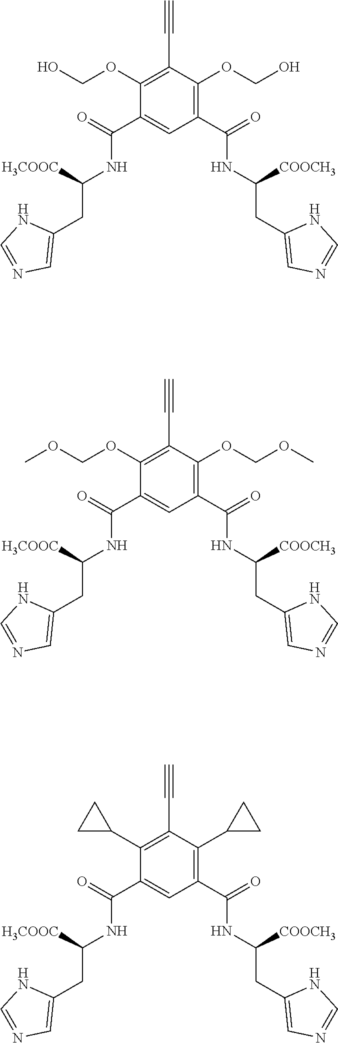

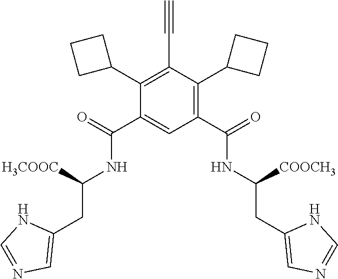

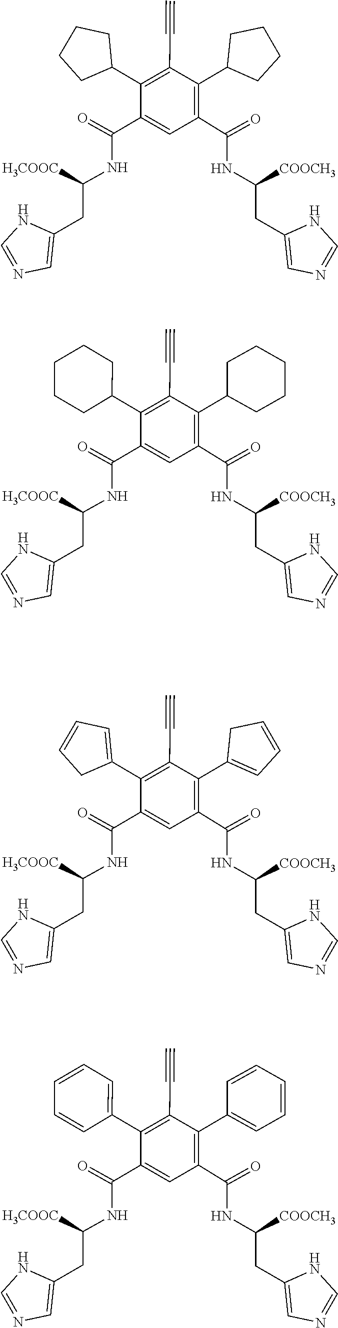

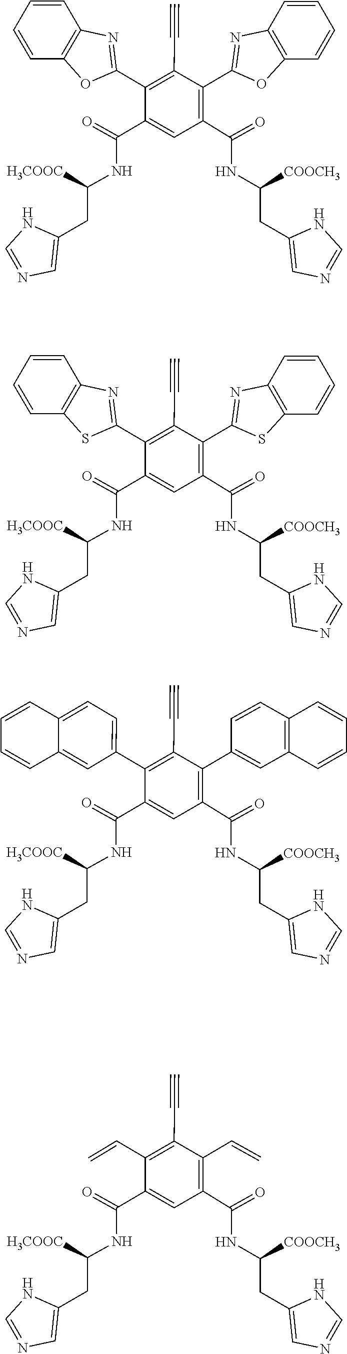

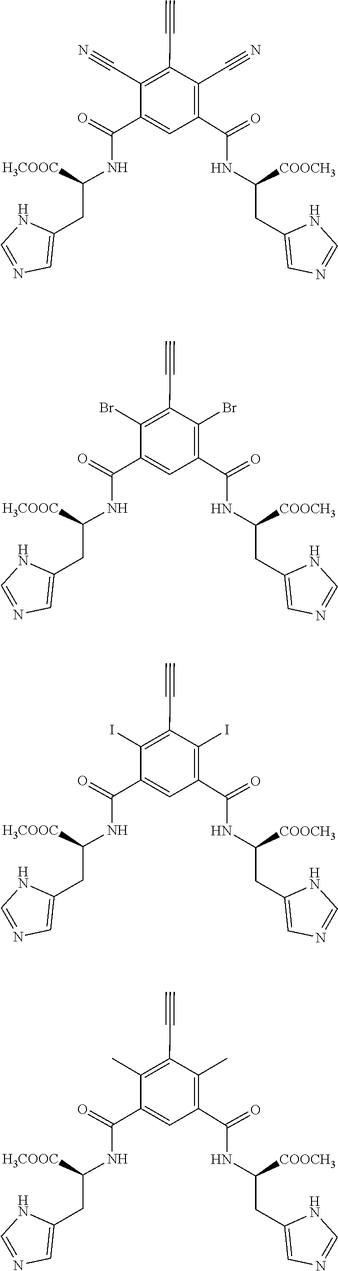

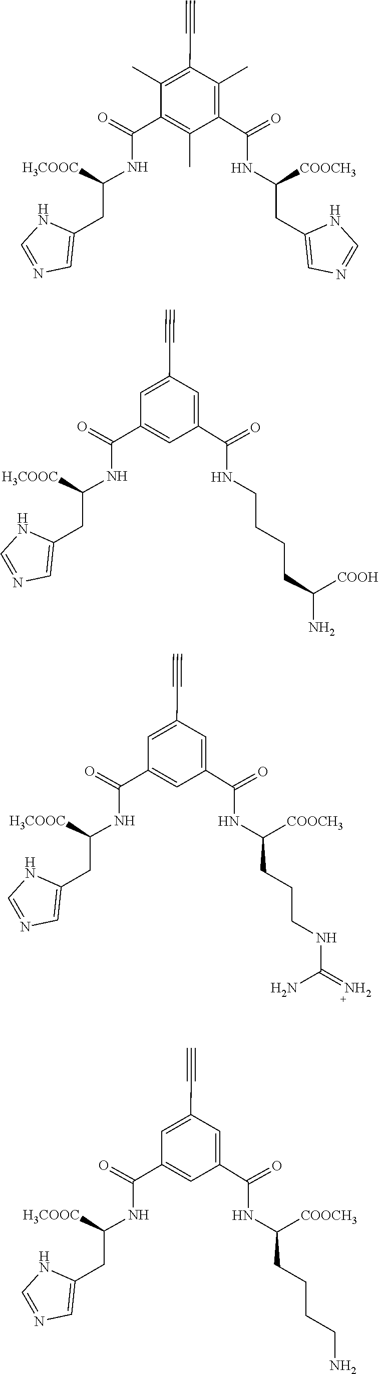

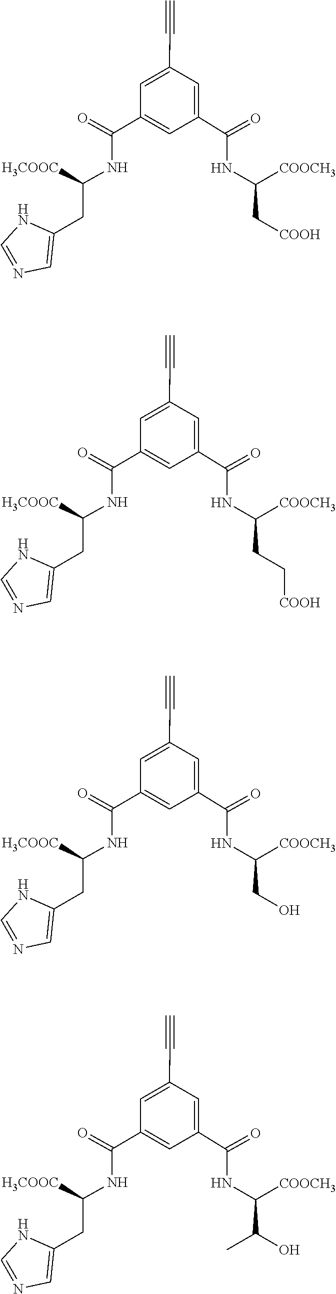

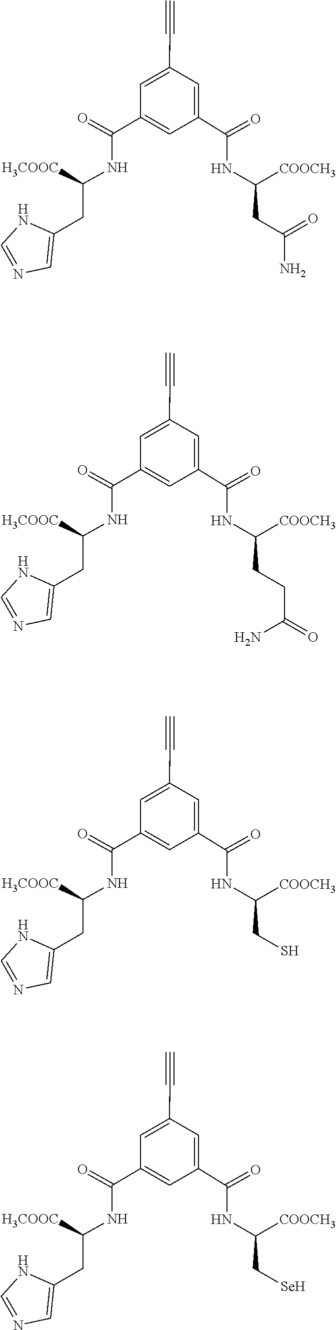

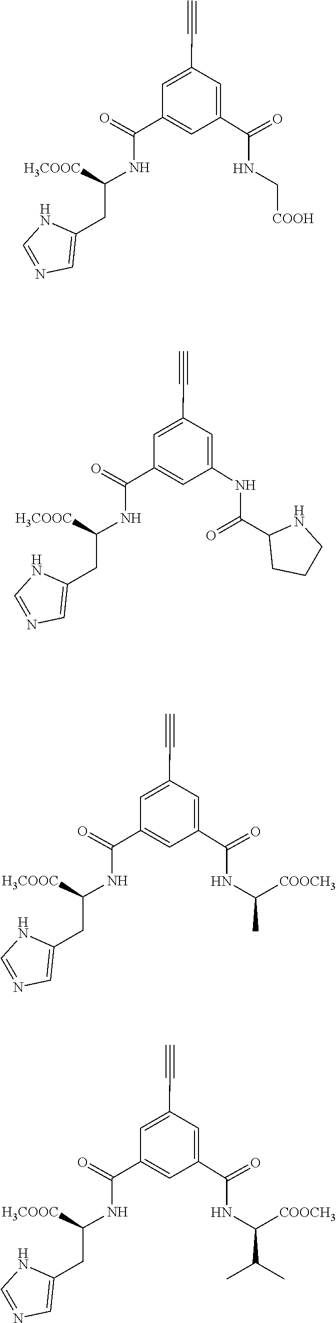

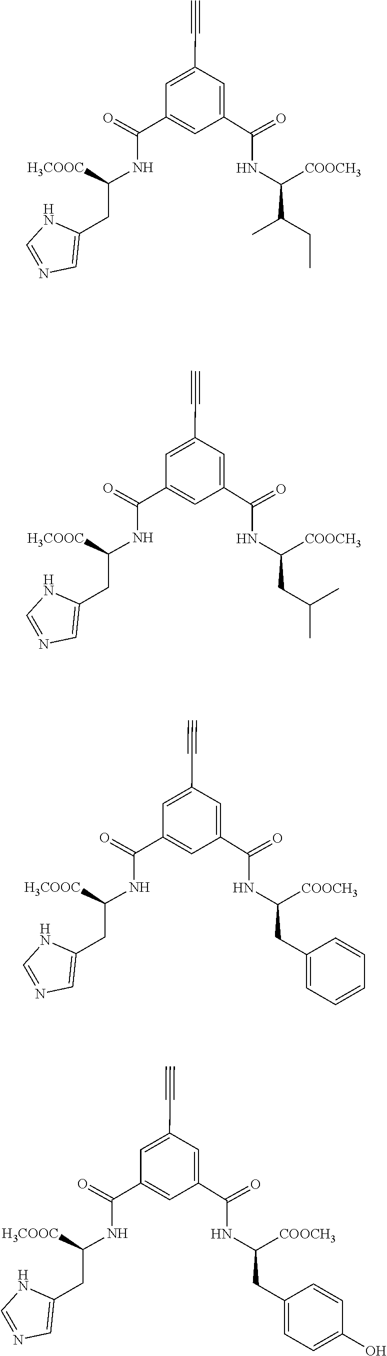

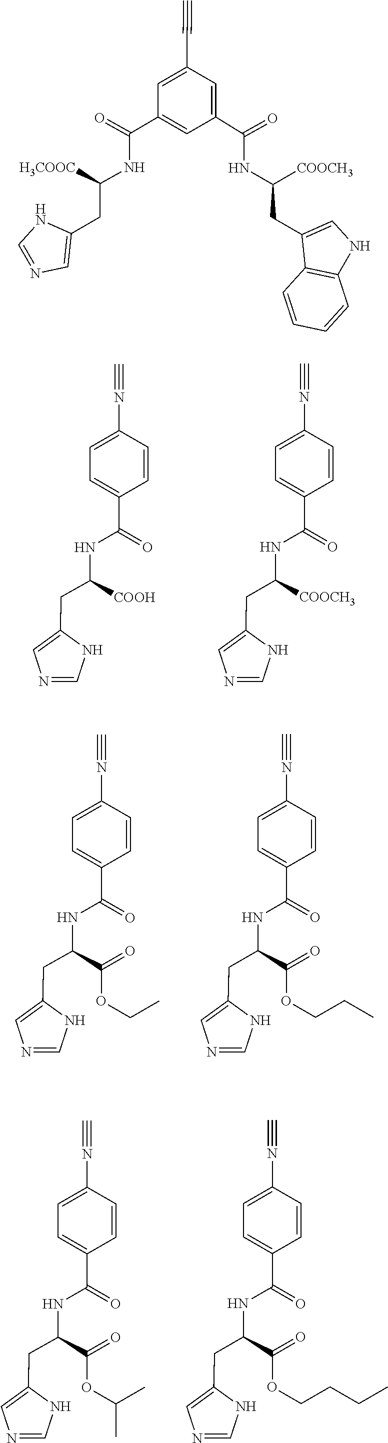

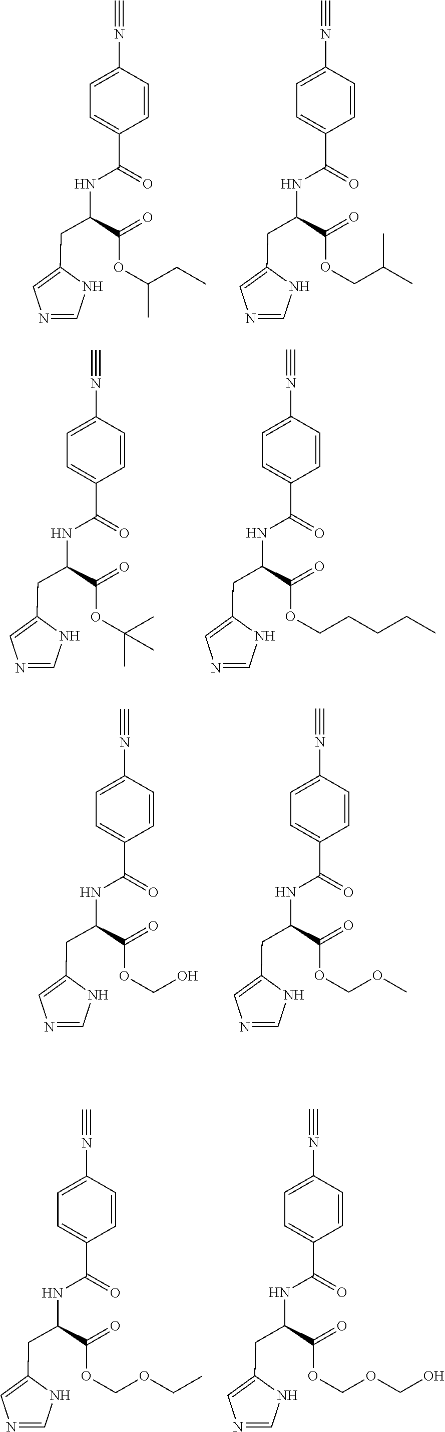

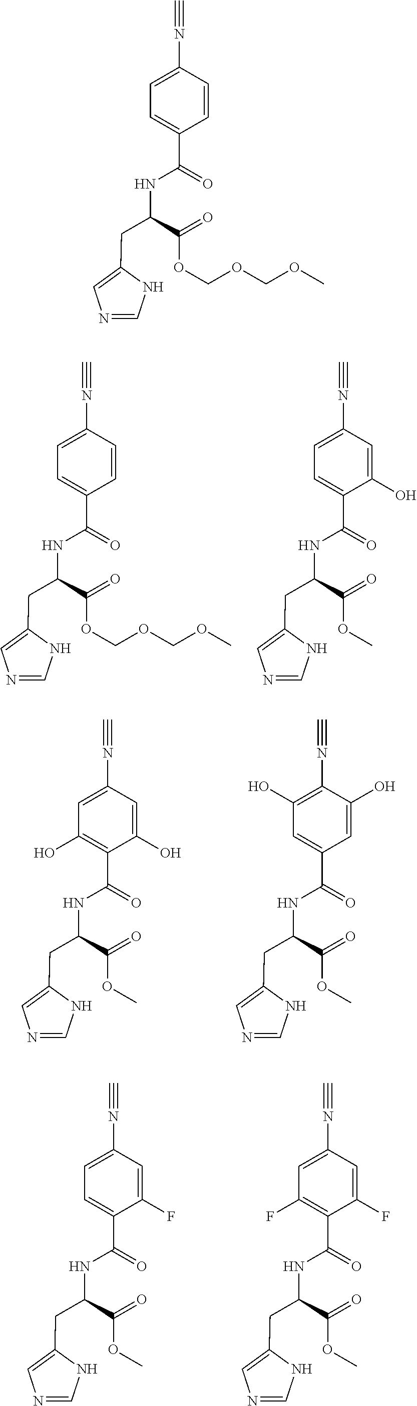

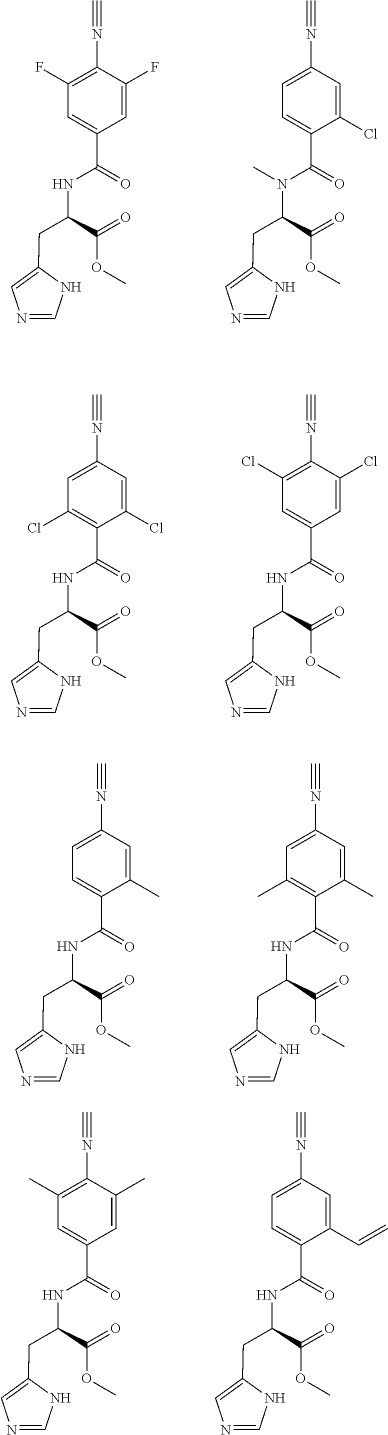

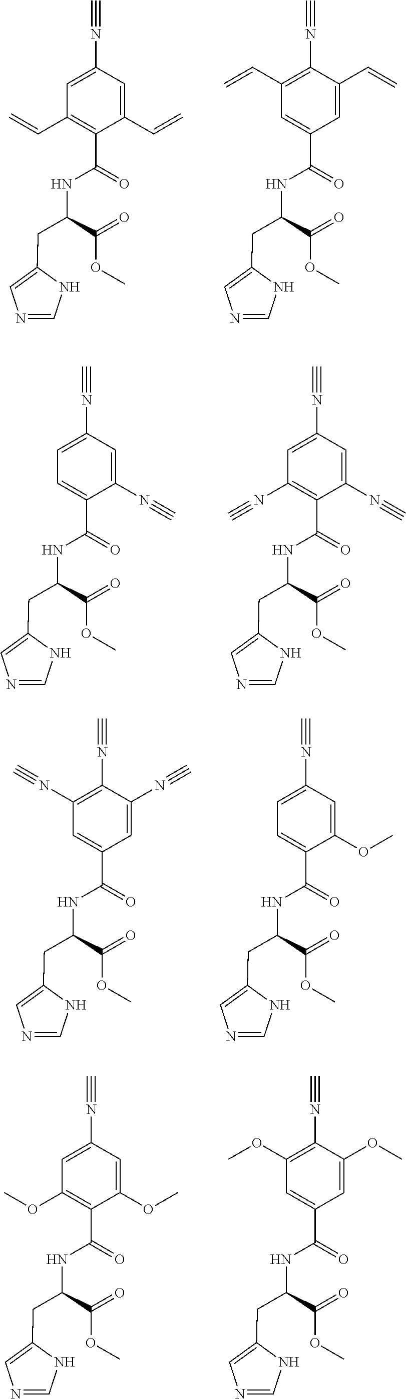

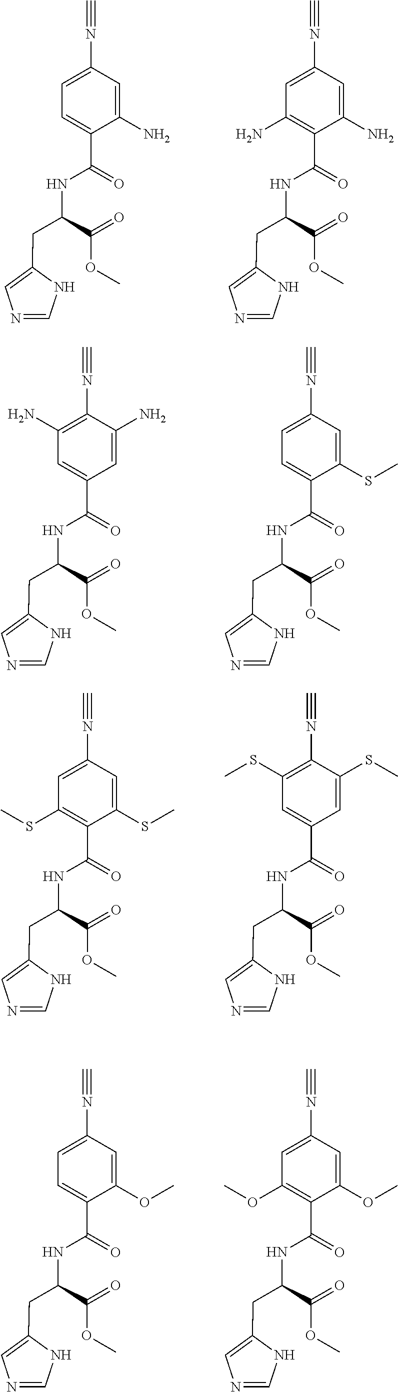

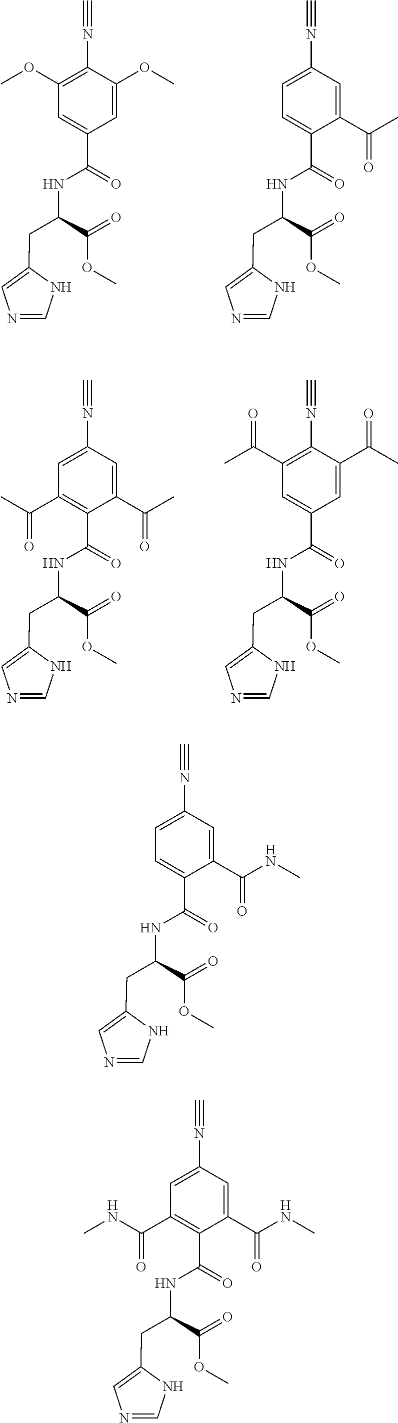

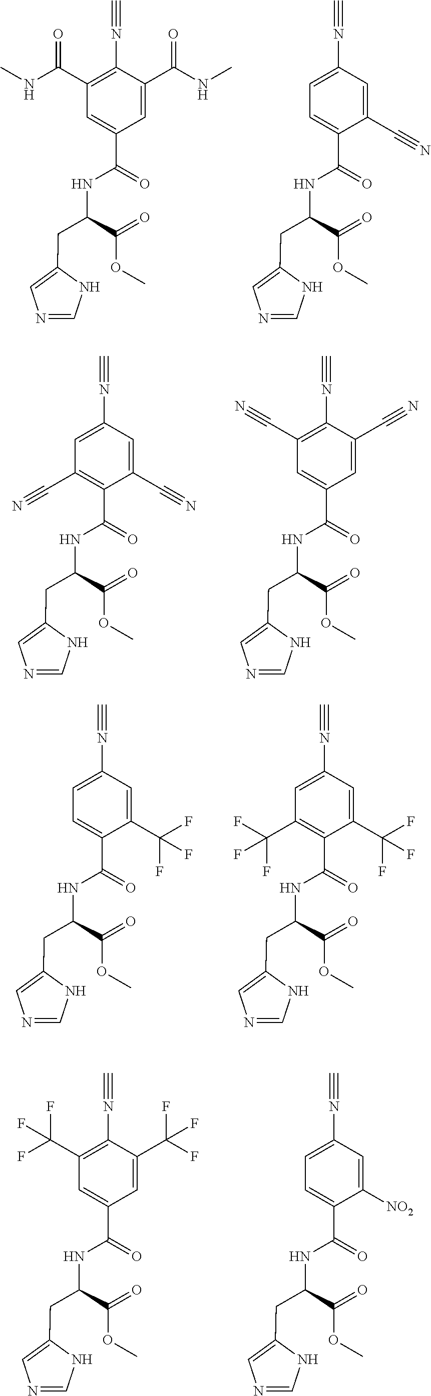

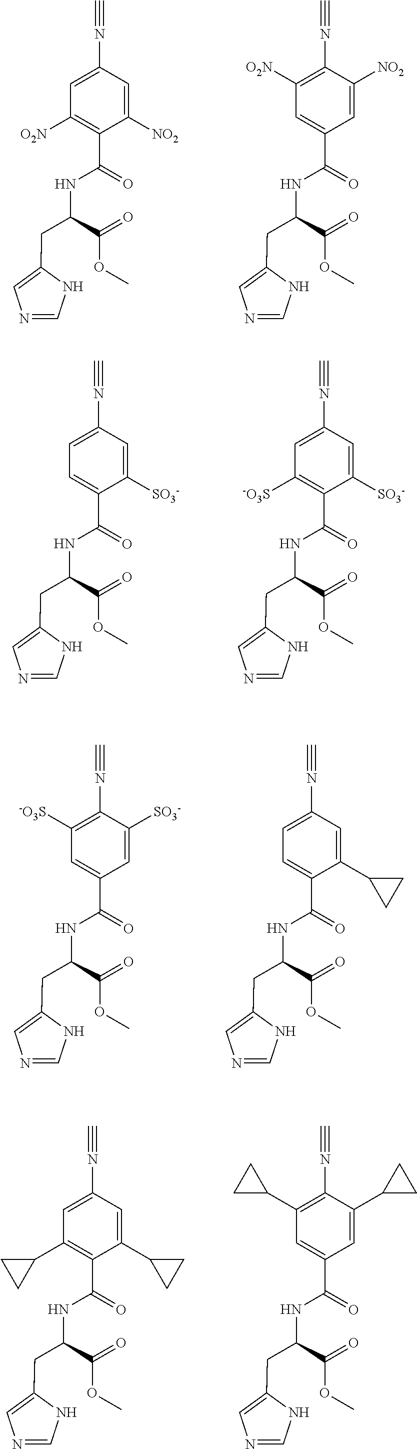

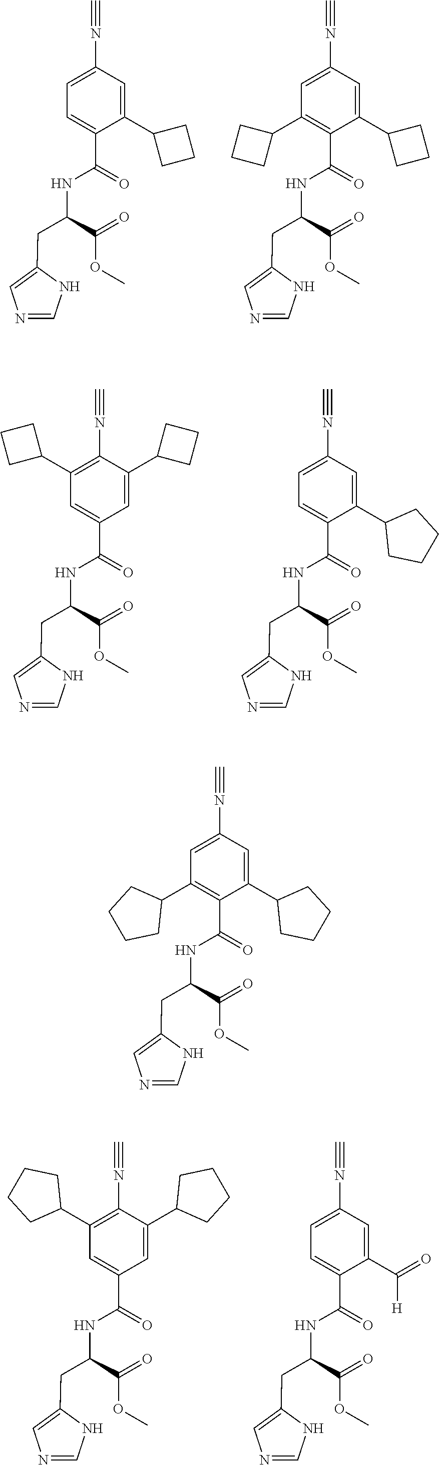

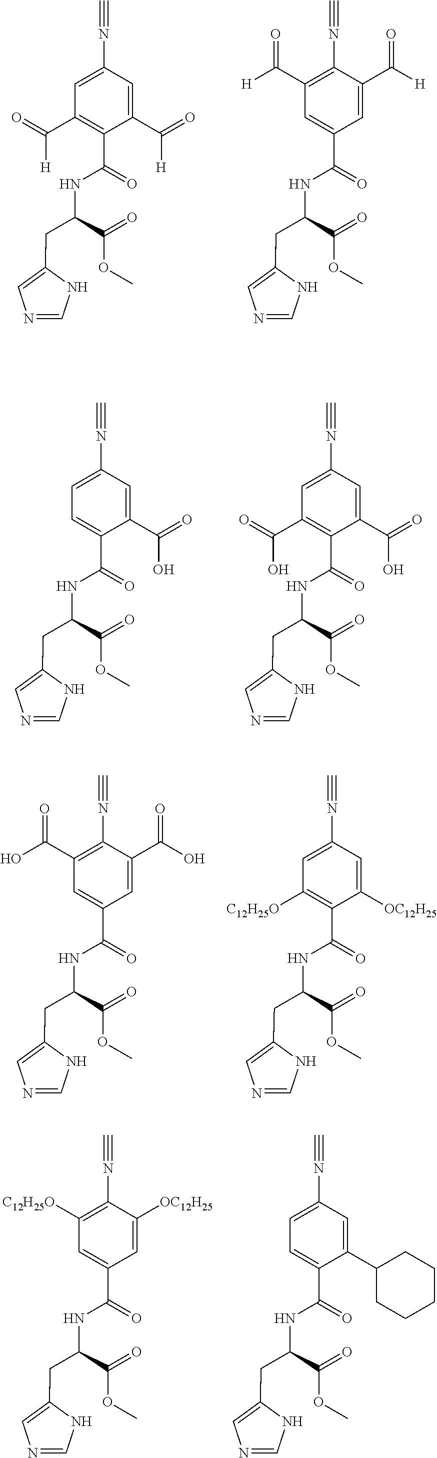

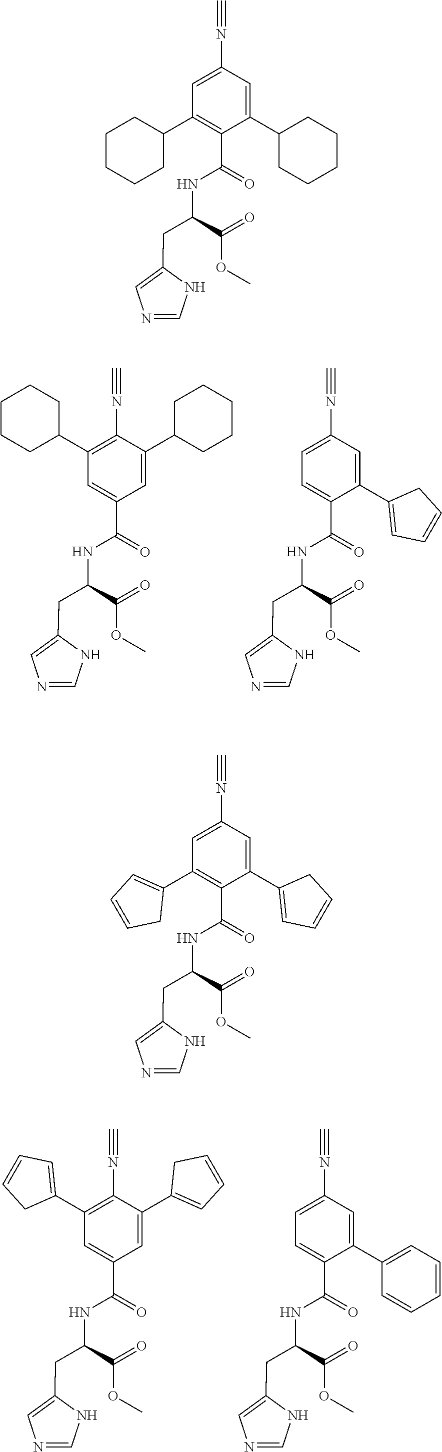

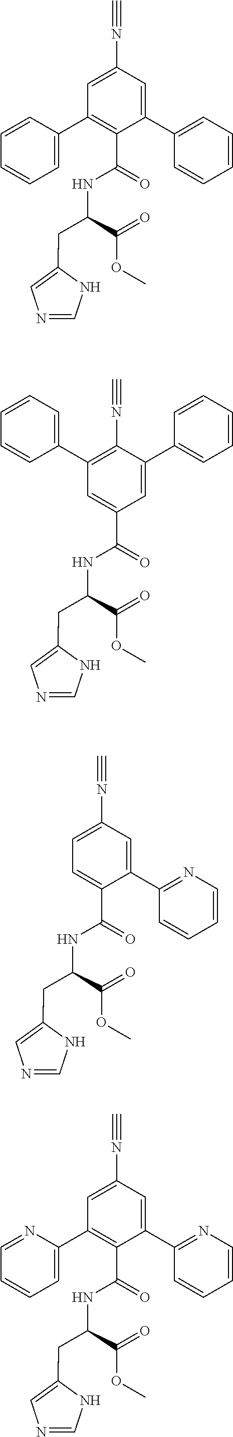

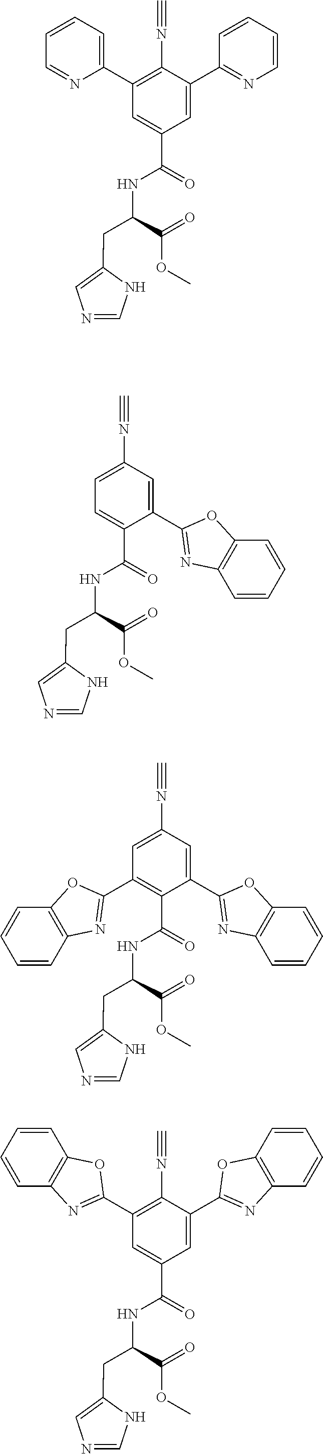

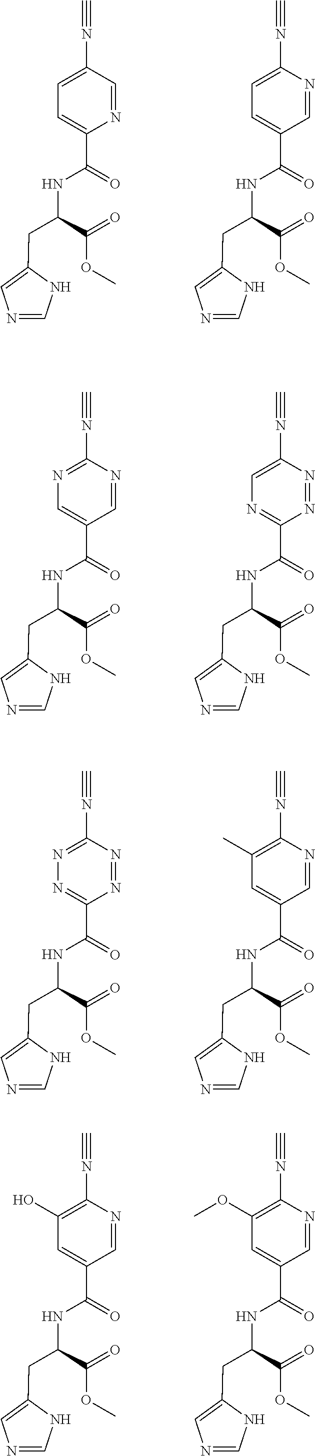

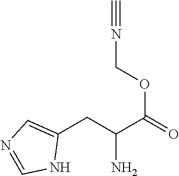

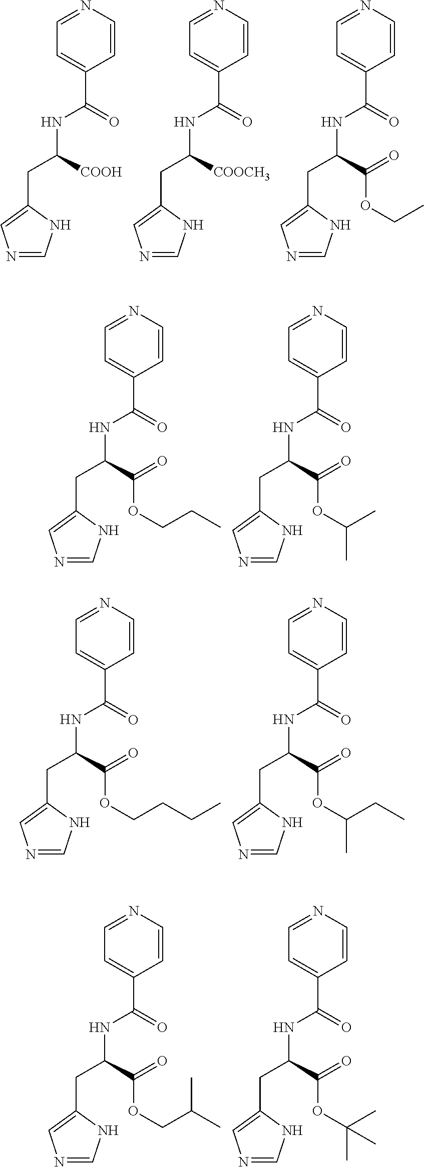

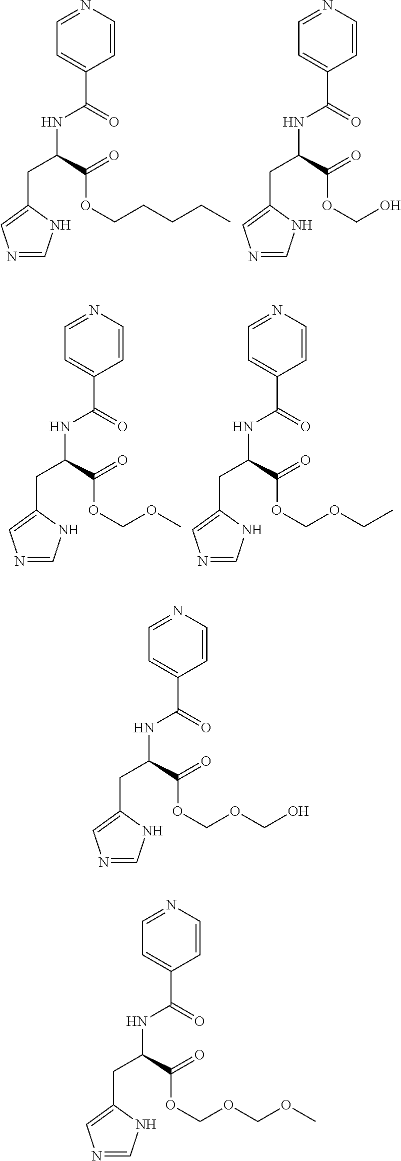

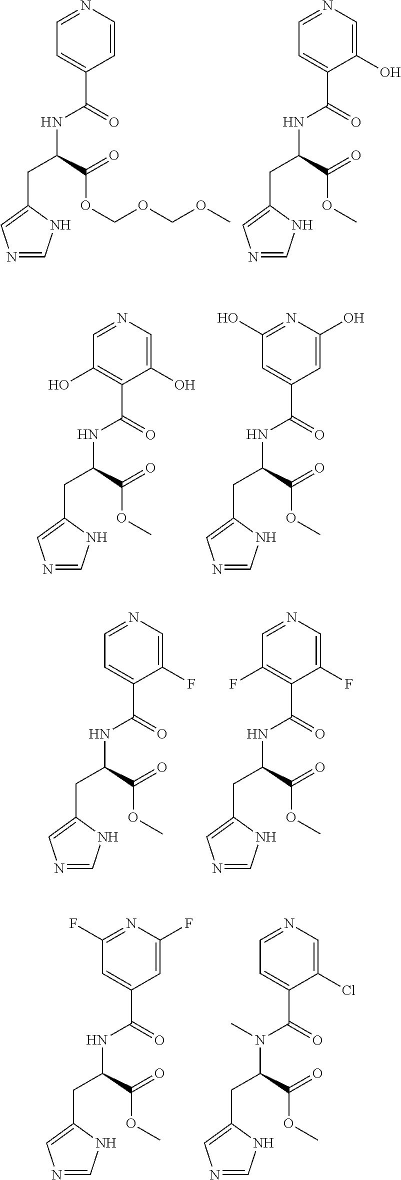

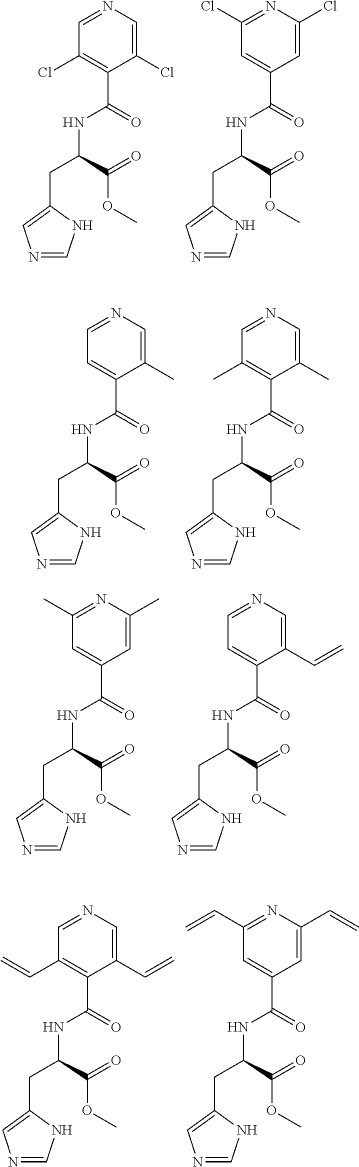

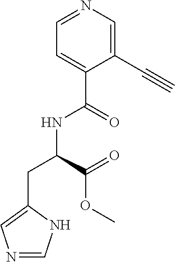 [SMILES: C#Cc1cnccc1C(=O)N[C@H](Cc1cnc[nH]1)C(=O)OC]

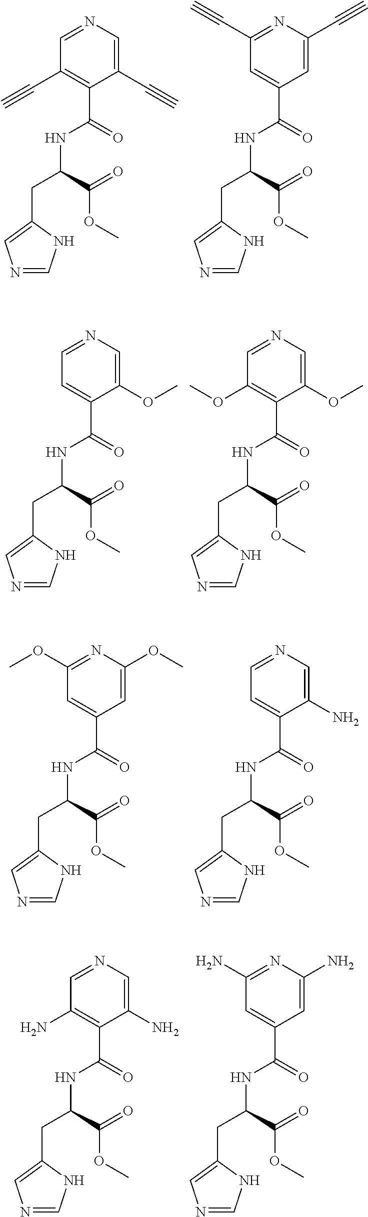

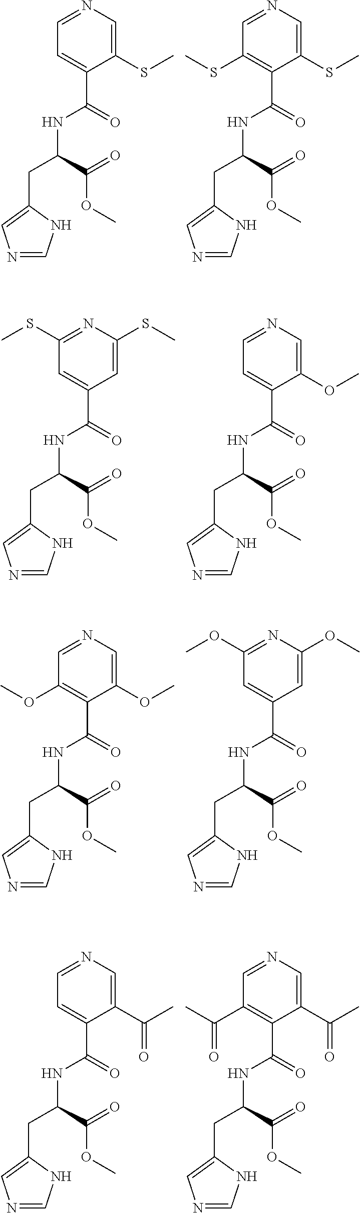

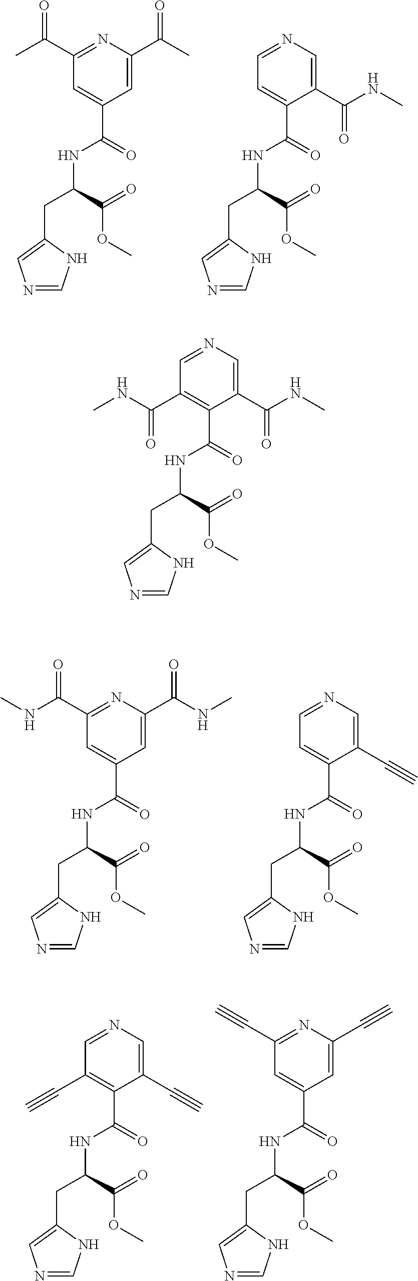

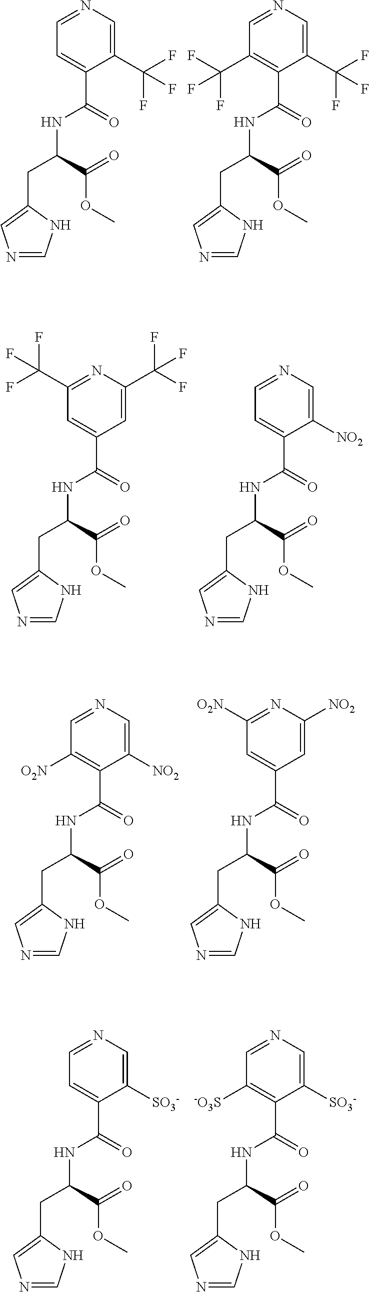

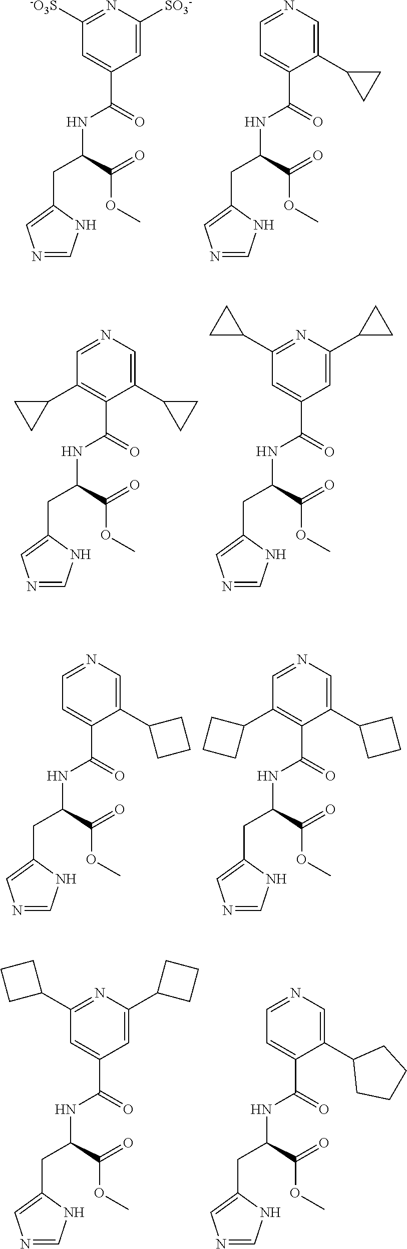

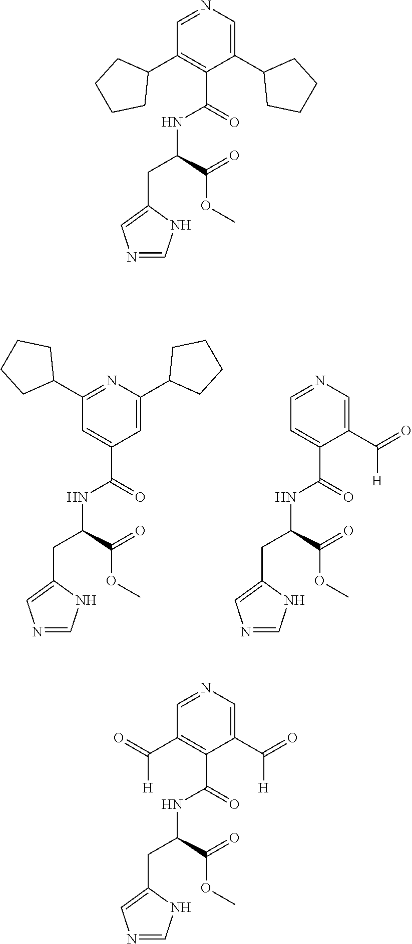 [SMILES: COC(=O)[C@@H](Cc1cnc[nH]1)NC(=O)c1c(C2CCCC2)cncc1C1CCCC1 ; COC(=O)[C@@H](Cc1cnc[nH]1)NC(=O)c1cc(C2CCCC2)nc(C2CCCC2)c1 ; COC(=O)[C@@H](Cc1cnc[nH]1)NC(=O)c1ccncc1C=O ; COC(=O)[C@@H](Cc1cnc[nH]1)NC(=O)c1c(C=O)cncc1C=O]

P represents positive charge-containing structure(s). The structures bearing positive charge can be selected from but not limited to substituted and unsubstituted amine, ammonium, pyridinium, pyrrodinium, phosphonium, imidazolium, sulfonium, and their derivatives. The exemplary structures of ligands in the Formula IV are shown below:

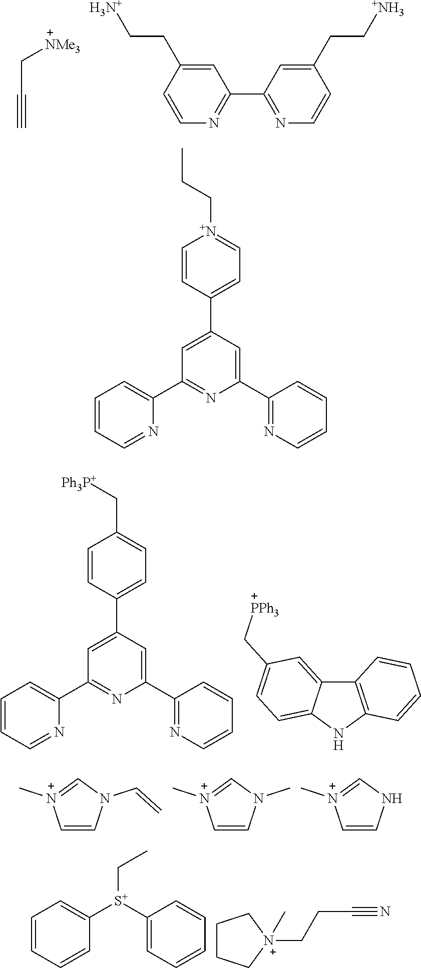

### C. Exemplary Formulae and Chemical Structures of the Compounds

Preferably, the metal complexes have a square-planar or partially planar geometry. The structure can be classified according to the type of ligands: monodentate, bidentate, tridentate, and tetradentate ligands. In some forms, the metal complex has a chemical structure shown by a generic Formula I:

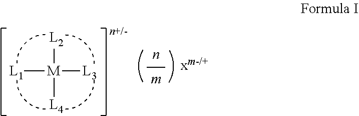

### Wherein:

(a) M is a metal atom (also known as metal center). It can be Pt(II), Pd(II), Ni(II), Ir(I), Rh(I), Au(III), Ag(III), and Cu(III).
(b) L₁, L₂, L₃, L₄ stand for ligands. Each ligand can offer one donor atom for coordination to the metal center.
(c) n+/− is the number of charge (positive or negative) which the metal complexes carry. n can be zero or a positive integer, for example, 1, 2, 3, 4, and 5.
(d) X is a counterion for charge neutrality of the compound. When X^{-/+} is an anion, for example, X^{m-}, it can be selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof.
   When X^{m-/+} is a cation, for example, X^{m+}, it can be selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof.
(e) m−/+ is the number of charge (positive or negative) which the counterions carry. X should carry a charge opposite to the charge of the metal complex. m can be zero or a positive integer, for example, 1, 2, 3, 4, and 5.
(f) n/m represents the stoichiometry of the counterions in the formula.
(g) Four dashed lines represent the optional independent covalent linking between two ligands, optional independent fusion of ring moieties from two ligands, or a combination thereof.
   The examples of Formula I are shown as follows:

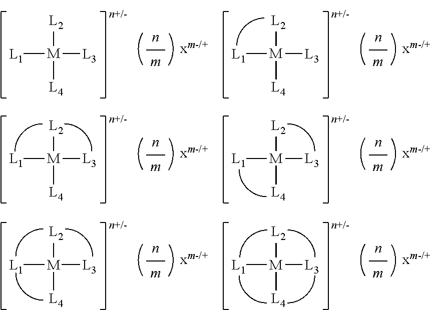

### Wherein:

(a) The lines stand for the optional linking between two ligands. They also represent the optional fusion of rings from different ligands.

In some forms, L₁, L₂, L₃, and L₄ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻—, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³. (R, R¹, R², and R³ would be defined above). For example, R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties).

In some forms, L₁, L₂, L₃ can be independently selected from C₆-C₅₀ arenes or C₃-C₅₀ heteroarenes. The structures include, but not limit to, five-membered arenes and their derivatives, such as furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; six-membered arenes and their derivatives, such as benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine.

In some forms, L₄ can be selected from five-membered arenes and their derivatives, such as furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; six-membered arenes and their derivatives, such as benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine; carbazole, dibenzothiophene, dibenzofuran, fluorene, halide, alkylamine, arylamine, alkylphosphine, arylphosphine, alkylarsine, arylarsine, SCN⁻, NO₃⁻, N₃⁻, O₂⁻, S₂⁻, H₂O, NO₂⁻, NCS⁻, NH₃, NO₂⁻, CN⁻, CO, R—C≡C—, RO⁻—, RS⁻—, RSe⁻—, and derivatives (wherein R is defined above). For example, R is selected from substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties).

In some forms, L₁ and L₂ are connected by covalent linkages, fusion of ring moieties from the two ligands, or a combination thereof. In some forms, L₂ and L₃ are further connected by covalent linkages, fusion of ring moieties from the two ligands, or a combination thereof. In some forms, L₁ and L₄ are further connected by covalent linkages, fusion of ring moieties from the two ligands, or a combination thereof. In some forms, L₃ and L₄ are further connected by covalent linkages, fusion of ring moieties from the two ligands, or a combination thereof.

In some forms, the metal complex has a chemical structure shown by a generic Formula II:

$${[ {L}_{5} — {M}^{′} — {L}_{6} ]}^{n + / -} ( \frac{n}{m} ) {X}^{m - / +} \qquad Formula II$$

### Wherein:

(a) M′ is a metal center screened from Ni(0), Pd(0), Pt(0), Cu(I), Ag(I), Au(I), Zn(II), Cd(II) and Hg(II).
(b) L₅ and L₆ stand for ligands. Each ligand can offer one donor atom for coordination to the metal center.

In some forms, the metal complex has a chemical structure shown by a generic Formula III, which exhibit a trigonal-planar geometry:

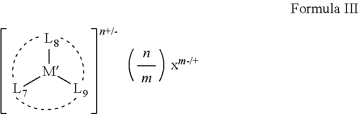

### Wherein:

(a) L₇, L₈, and L₉ stand for ligands. Each ligand can offer one donor atom for coordination to the metal center.

The examples of Formula III are shown as follows:

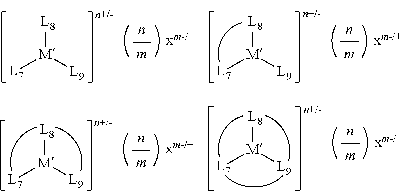

### Wherein:

(a) The lines stand for the optional linking between two ligands. They also represent the optional fusion of rings from different ligands, or a combination thereof.

In some forms, the metal complex has a chemical structure shown by a generic Formula V:

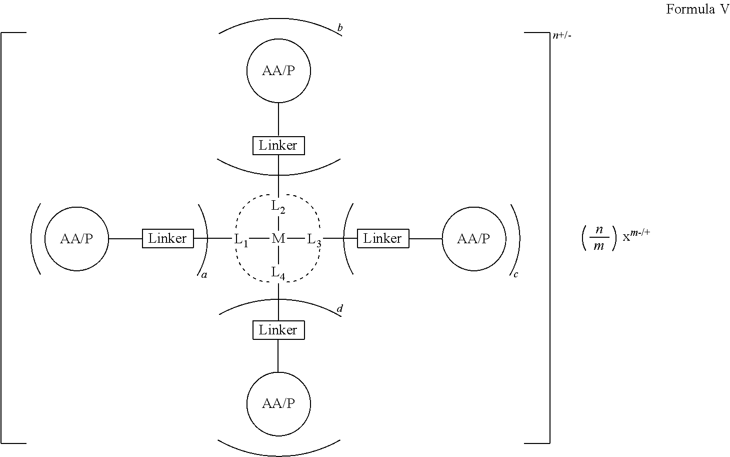

### Wherein:

(a) a, b, c, d are independently 0 or a positive integer, such as 1, and a+b+c+d>0.
(b) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and combination thereof.
(c) AA are selected from amino acids, their derivatives, or combination of amino acids and/or their derivatives, preferably AA are selected from:

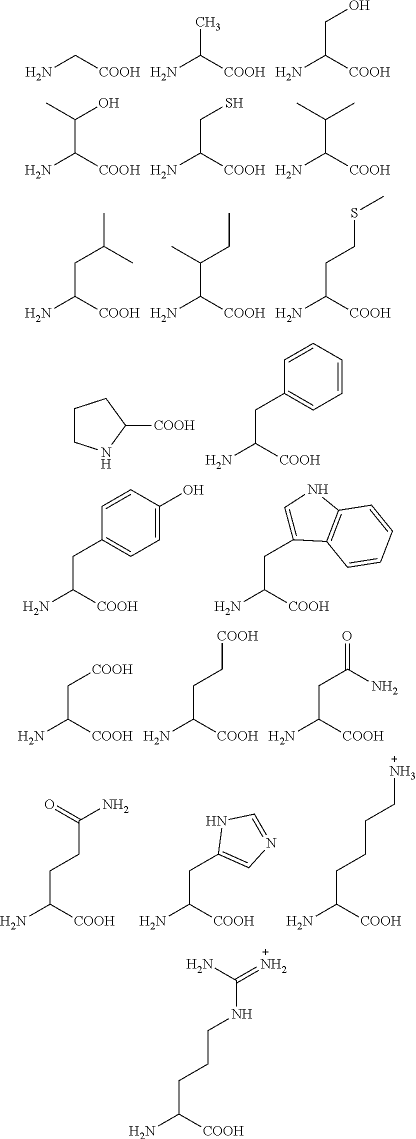

(d) P represents positive charge-containing structure(s), preferably substituted and unsubstituted amine, ammonium, pyridinium, pyrrodinium, phosphonium, imidazolium, sulfonium, and their derivatives.

In some forms, the metal complex is as described above, except that the metal complex has a chemical structure shown by a generic Formula VI:

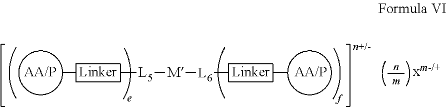

### Wherein:

(a) e, f are independently 0 or a positive integer, such as 1, and e+f>0.

In some forms, the metal complex is as described above, except that the metal complex has a chemical structure shown by a generic Formula VII:

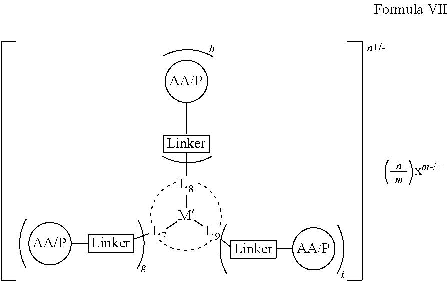

wherein:
(a) g, h, i are independently 0 or a positive integer, such as 1, and g+h+i>0.
   Examples of metal complexes designed as formulae I, II, III, V, VI, and VII are shown below.

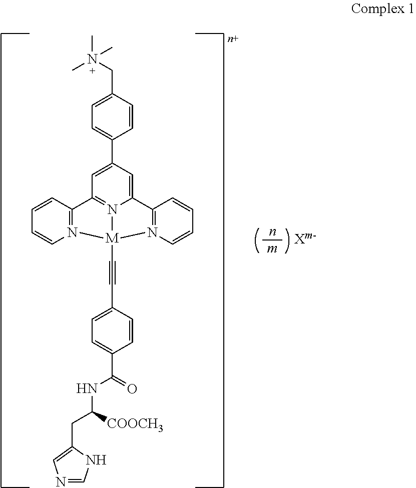

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n stands for the stoichiometry of the counterions in the formula.

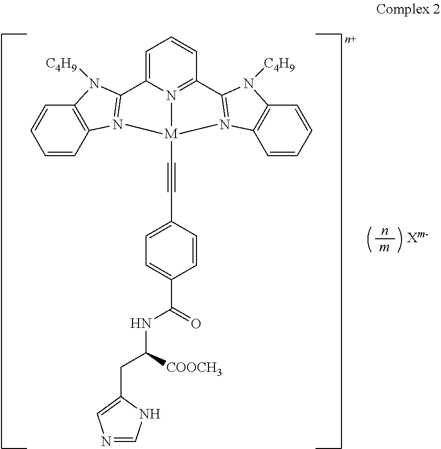

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

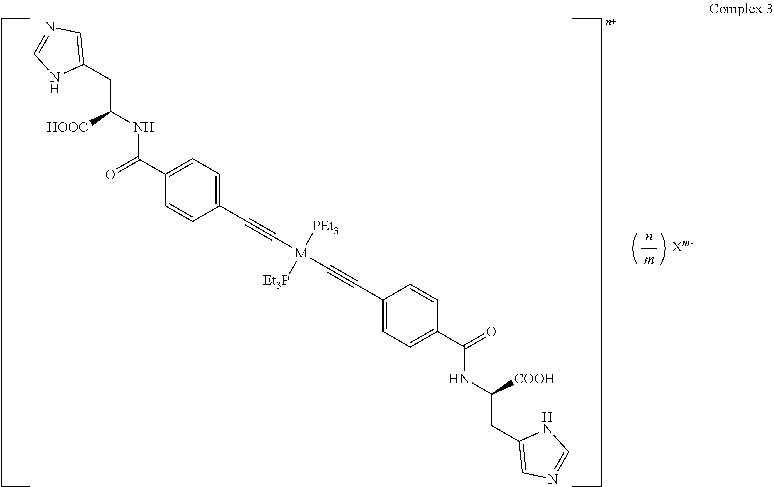

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

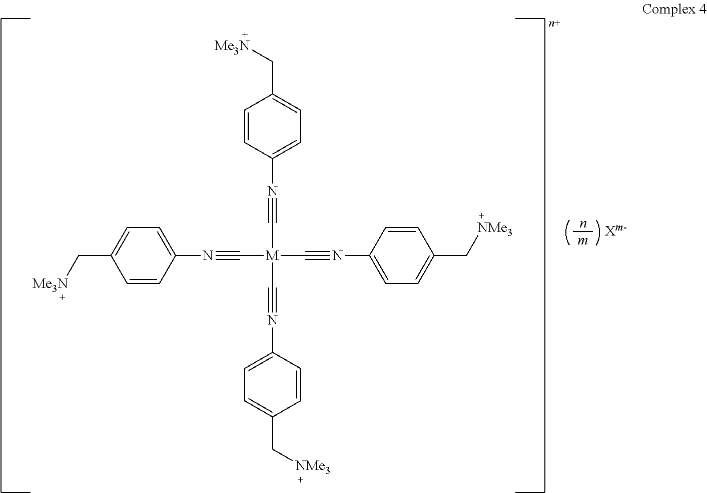

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

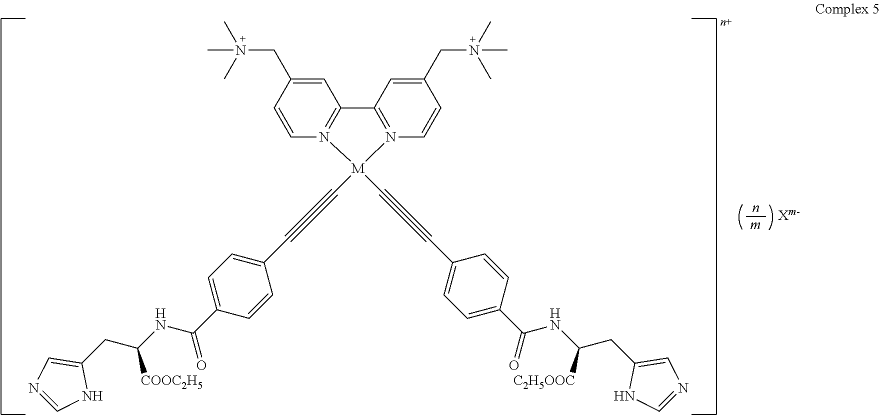

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

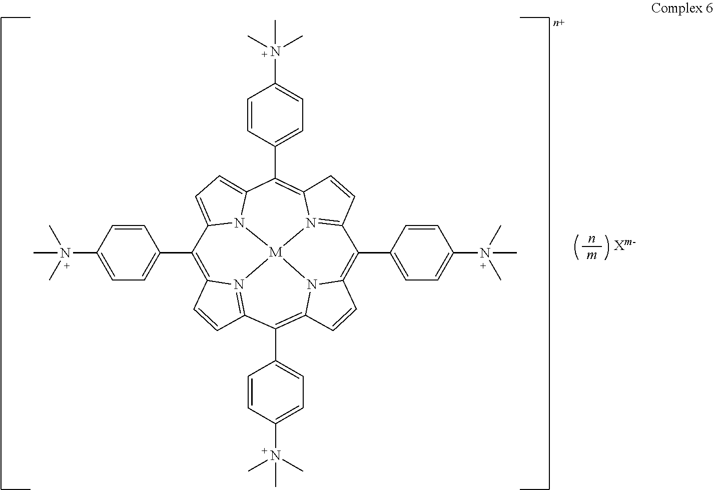

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

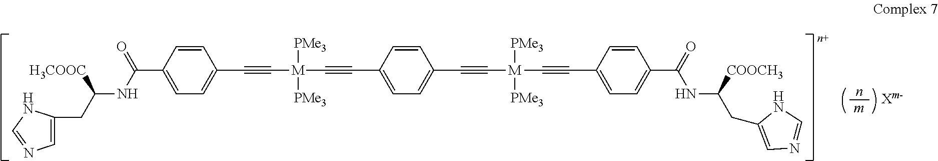

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

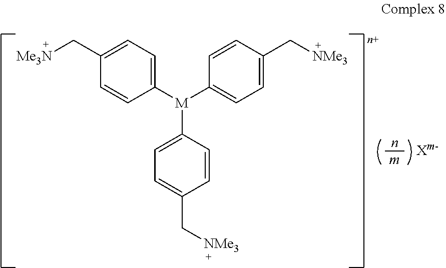

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

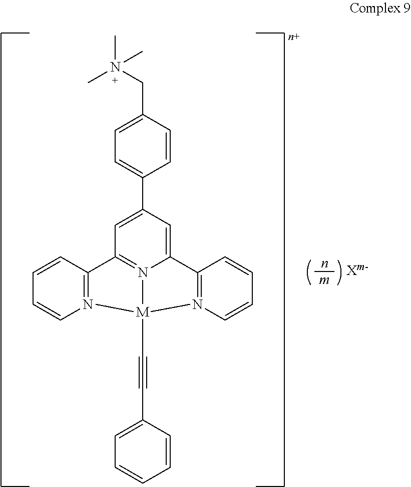

Wherein:
(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

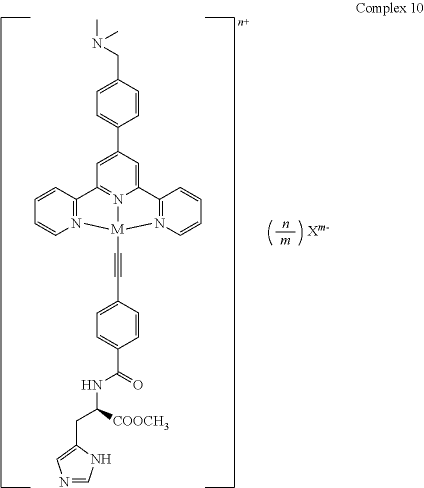

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

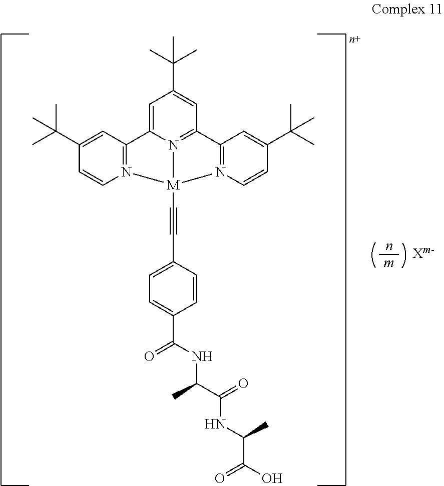

### Wherein:

(a) M stands for metal center, which can be selected from Ir(I), Rh(I), Pt(II), Pd(II), Ni(II), Au(III), Ag(III), or Cu(III).
(b) n represents the number of charges of the metal complex in the formula, wherein n is zero or a positive integer.
(c) X represents the counterion for charge neutrality, wherein n is zero or a positive integer, m=n or m≠n.
(d) n/m stands for the stoichiometry of the counterions in the formula.

### D. Mixtures and Compositions

Also disclosed are mixtures containing a series of compounds. The mixtures can be used to detect and/or image the analyte and/or differentiate cancer cells from normal cells, and/or screen the efficacy of inhibitors. For example, the analyte can be sialic acids, polysialic acids, glycans and cancer cells.

In some forms, the chemical structures of compounds can be exhibited using Formulae I, II, III, VI, V, VI, or VII.

In some forms, the photophysical properties of the compounds exhibit clear changes upon addition of sialic acids, polysialic acids, glycans, or cancer cells. In some forms, the compounds would not be interfered with in the presence of other monosaccharides. In some forms, the compounds can image sialic acids on the surface of cancer cells and exhibit different luminescence signals on the surface of cancer cells and normal cells, realizing the differentiation of different types of cells. In some forms, the compounds can be used for early diagnosis of cancer and/or diseases and anticancer therapies.

### E. Kits

The kits can include the above compounds, mixtures, and compositions.

The kits include one or more containers, one or more disclosed compounds, mixtures, and compositions. The kits contain other components, such as compounds, solutions, materials, and carriers. The materials of carriers would not interfere with the effectiveness of the disclosed compounds. The kits can include instructions for use.

The kits can be used to detect the analyte and/or differentiate cancer cells from normal cells and screen the efficacy of inhibitors.

The kits can also include one or more positive controls and negative controls.

### III. Methods of Making and Reagents Therefor

The compounds of Formulae I, II, III, VI, V, VI, or VII can be readily synthesized using techniques generally known to synthetic organic and inorganic chemists. Exemplary methods to synthesize a specific compound of Formulae I, i.e., complex 1-Pt, are described in the disclosed examples.

### IV. Methods of Using

The disclosed compounds can be used in a method to sense and/or image an analyte, differentiate cancer cells from normal cells, detect or diagnose cancer and/or diseases and screen and test the inhibitors.

In some forms, the analytes can be glycans, such as sialic acids, polysialic acids, etc., and cancer cells.

A method for sensing and/or imaging analyte in a sample (preferably a biological sample) can be: (1) adding one or more compounds into a container, and then adding the sample or vice versa, followed by mixing the complex with the sample; (2) measuring the changes in the photophysical properties of the complex to check whether assembly/disassembly of the complex occurs, which was induced by combining the sample and the one or more compounds in the container. The variation in the photophysical properties indicates the presence of the analyte of interest.

A method for testing the efficacy of inhibitors to remove analytes or inhibit the generation of analytes can be: (1) mixing the compounds disclosed herein, such as compounds of Formulae I, II, and/or III with the inhibitor-treated samples (preferably biological samples), and then mixing the compounds with the corresponding samples (preferably the corresponding biological sample) that are not to be treated with the inhibitor; (2) measuring the changes in the photophysical properties of the complex to check whether assembly/disassembly of the complex occurs, which was induced by combining the sample and the one or more compounds. The variation in the photophysical properties indicates the efficacy of inhibitors. Weak inhibitors do not significantly reduce the level of sialic acids, resulting in a relatively high concentration of sialic acids on the surface of cancer cells. Such high levels of sialic acid can still induce self-assembly and aggregation, causing dramatic changes in photophysical properties. In contrast, strong inhibitors that facilitate the reduction of sialic acid on the cellular surface can prevent self-assembly and aggregation of compounds and even lead to deaggregation of compounds. The distinct variation in photophysical properties resulting from the use of inhibitors with different efficiency can be utilized to evaluate the efficiency of inhibitors. This can be used to screen highly effective inhibitors for therapies or other applications. The kits are not only confined to solution kits used in spectroscopic measurements or observation of color changes or luminescence changes in the solution state but can also be modified or extended to indicator test on a solid substrate indicator plate or an indicator paper.

### A. Sensing or Visualizing Sialic Acids, Polysialic Acids, Glycans or Cancer Cells.

Also disclosed are methods to detect and/or image glycans, such as sialic acids, polysialic acids, etc., and cancer cells. The methods can be: (1) adding one or more of the disclosed compounds into a container, and then adding a sample (preferably biological sample), or vice versa, followed by mixing the compound with the sample; (2) measuring the changes in the photophysical properties (such as color, absorbance, luminescence intensity, circular dichroism, circularly polarized luminescence, or combinations thereof) of the complex to check whether assembly/disassembly of the complex occurs, which was induced by combining the sample and one or more compounds in the container. The variation in the photophysical properties indicates the changes of the self-assembly behavior of the complex, suggesting the presence of sialic acids, polysialic acids, glycans, or cancer cells in the sample.

According to the types of photophysical properties, the measurement methods can be luminescence emission assays, UV-vis absorption assays, luminescence lifetime assays, circular dichroism, circularly polarized luminescence, or combinations thereof.

For example, the detection toward polysialic acids can be conducted via a luminescence assay as shown in FIG. 5. Upon addition of polysialic acids, the increasing level of self-assembly and/or aggregation of the metal complex can be indicated by an increase or growth of an emission band. The metal complex can bind to polysialic acids via electrostatic interaction and hydrogen bonding, resulting in the supramolecular self-assembly of the metal complex. The changes in the color and/or luminescence of the metal complex can be used as an indicator of polysialic acids.

The detection can be conducted in cuvettes, multi-well plates using a non-imaging spectrometer. The detection can also be conducted in cell dishes, multi-well plates using a imaging spectrometer. The kits are not only confined to solution kits used in spectroscopic measurements or observation of color changes or luminescence changes in the solution state but can also be modified or extended to indicator test on a solid substrate indicator plate or an indicator paper.

B. Differentiating Cancer Cells from Normal Cells

Also disclosed are methods to differentiate cancer cells from normal cells. The methods can be: (1) mixing one or more of the compounds with a sample (preferably biological sample), (2) imaging the changes in the photophysical properties of the complex which is related to the changes in the degree of supramolecular self-assembly of the complex. The variation in the photophysical properties indicates the presence of sialic acids on the cellular surface. The high levels of sialic acids on the surface of cancer cells can facilitate the differentiation of cancer cells and detection and/or diagnosis of cancers/tumors and/or diseases, and for imaging-guided surgery. The number of sialic acid molecules on the cellular surface of cancer cells can range from 10⁴-10¹¹, 10⁵-10¹¹, 10⁶-10¹¹, or 10⁷-10¹¹ molecules, particularly 10⁷-10¹¹ molecules, which can be regarded as a high level. The reported luminescent assays have reported higher levels of sialic acids in cancer cells compared to normal cells. The high density of these sialic acids leads to the self-assembly and aggregation of compounds that target them in cancer cells (Wang, et al., Anal. Chem., 2017, 89, 538-543; Xu, et al., Talanta, 2020, 209, 120579).

### C. Screening the Inhibitors Against Sialic Acids, Polysialic Acids, and Glycans

Also disclosed is a method for testing the efficacy of inhibitors to remove analytes or inhibit the generation of analytes. It can be: (1) mixing one or more of the disclosed compounds with the inhibitor-treated samples (preferably biological samples), and then mixing the compounds with the corresponding samples (preferably the corresponding biological samples) that are not treated as a control group; (2) measuring the changes in the photophysical properties of the complex to check whether the supramolecular self-assembly degree of the complex changes. The variation in the photophysical properties indicates the changes of the self-assembly behavior of the complex, suggesting the efficacy of inhibitors. This can be used to screen useful inhibitors to remove sialic acids, which can be used for anticancer therapy. The kits are not only confined to solution kits used in spectroscopic measurements or observation of color changes or luminescence changes in the solution state but can also be modified or extended to indicator test on a solid substrate indicator plate or an indicator paper.

### D. Combinational Use

Also disclosed is a combinational use of more than one of the disclosed compounds. Mixtures containing different compounds can exhibit separate specificities toward various glycans, including sialic acids, polysialic acids, etc. The different variation in the photophysical properties from different compounds in the mixtures can be utilized to sense different components in samples (preferably biological samples). Also, the mixtures can be used to image or visualize, and/or monitor the level of glycans, such as sialic acids, polysialic acids, etc.

### E. Samples

In some forms, the samples (preferably biological samples) contain glycans, such as sialic acids, polysialic acids, and/or other monosaccharides, etc. The samples can be body fluid (such as blood, plasma, serum), cells (such as eukaryotic cells optionally selected from 3T3 cells, HeLa cells, HepG2 cells, MCF7 cells, HEK293T cells, Chinese hamster ovary (CHO) cells, and other cells), tissues (such as brain tissue, heart tissue, liver tissue, kidney tissue, spleen tissue, lung tissue, etc.), and animals.

The disclosed compositions and methods can be further understood through the following paragraphs.
1. A compound for detecting and/or imaging an analyte, wherein the compound is a d⁸ or d¹⁰ metal complex or a salt thereof, comprising: (a) a metal atom (also known as metal center) with a coordination number of 2, 3, or 4, selected from the group composed of Pt(II), Pd(II), Ni(II), Ir(I), Rh(I), Au(III), Ag(III), Cu(III), Ni(O), Pd(O), Pt(O), Cu(I), Ag(I), Au(I), Zn(II), Cd(II), and Hg(II); and
(b) one or more ligands with donor atoms independently selected from the group consisting of carbon (C), nitrogen (N), oxygen (O), phosphorus (P), sulfur (S), arsenic (As), and selenium (Se),
   wherein the metal complex binds to the analyte, wherein binding of the metal complex to the analyte induces supramolecular self-assembly and/or aggregation of the metal complex through noncovalent metal-metal and/or π-π interactions.
2. The compound of paragraph 1, wherein the compound has a structure of Formula I:

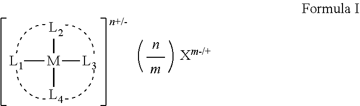

Wherein:
   (a) M is a metal atom (also known as metal center). It can be Pt(II), Pd(II), Ni(II), Ir(I), Rh(I), Au(III), Ag(III), and Cu(III);
   (b) L₁, L₂, L₃, L₄ stand for ligands. Each ligand can offer one donor atom for coordination to the metal center;
   (c) n+/− is the number of charge (positive or negative) which the metal complexes carry, where n can be zero or a positive integer, for example, 1, 2, 3, 4, and 5;
   (d) X is a counterion for charge neutrality of the compound. When X^{m-/+} is an anion, for example, X^{m-}, it can be selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄⁻²), hydrogenphosphate (H₂PO₄²⁻), phosphate (PO₄³⁻), and derivatives thereof. When X^{m-/+} is a cation, for example, X^{m+}, it can be selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
   (e) m−/+ is the number of charge (positive or negative) which the counterions carry. X should carry a charge opposite to the charge of the metal complex. m can be zero or a positive integer, for example, 1, 2, 3, 4, and 5;
   (f) n/m represents the stoichiometry of the counterions n in the formula; and
   (g) Four dashed lines represent the optional independent covalent linking between two ligands, optional independent fusion of ring moieties from two ligands, or a combination thereof.
3. The compound of paragraph 2, wherein L₁, L₂, L₃, and L₄ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene; halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻, RO⁻, RS⁻, RSe⁻, N═N═N—R, N≡C—R wherein N is a donor atom, C≡N—R wherein C is a donor atom, NR¹R²R³, PR¹R²R³, and AsR¹R²R³. (R, R¹, R², and R³ would be defined above). For example, R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties).
4. The compound of paragraph 2 or paragraph 3 wherein L₁ and L₂ are connected by covalent linkages, fusion of ring moieties from the two ligands, or a combination thereof.
5. The compound of paragraph 1, wherein the compound has a structure of Formula II:

$${[ {L}_{5} — {M}^{′} — {L}_{6} ]}^{n + / -} ( \frac{n}{m} ) {X}^{m - / +} \qquad Formula II$$

Wherein:
   (a) M′ is a metal center selected from Ni(0), Pd(0), Pt(0), Cu(I), Ag(I), Au(I), Zn(II), Cd(II) and Hg(II); and
   (b) L₅ and L₆ stand for ligands. Each ligand can offer one donor atom for coordination to the metal center.
6. The compound of paragraph 1, wherein the compound has a structure of Formula III:

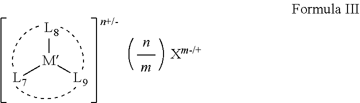

Wherein:
   (a) L₇, L₈, and L₉ stand for ligands. Each ligand can offer one donor atom for coordination to the metal center.
7. The compound of any one of paragraphs 1-6, wherein the ligand has a structure of Formula IV:

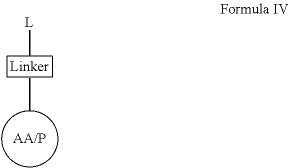

Wherein:
   (a) L stands for chemical moieties containing one or more donor atoms, preferably one donor atom, for coordination to the metal center of the metal complexes;
   (b) Linker stands for structures that facilitate the optional covalent linking moieties between L and amino acid/positive-charge containing structure(s);
   (c) AA stands for amino acids, or their derivatives, or combination of amino acids and/or their derivatives. thereof; and
   (d) P represents positive charge-containing structure(s), preferably substituted and unsubstituted amine, ammonium, pyridinium, pyrrodinium, phosphonium, imidazolium, sulfonium, and their derivatives.
8. The compound of any one of paragraphs 1-7, wherein the compound has a structure of Formula V:

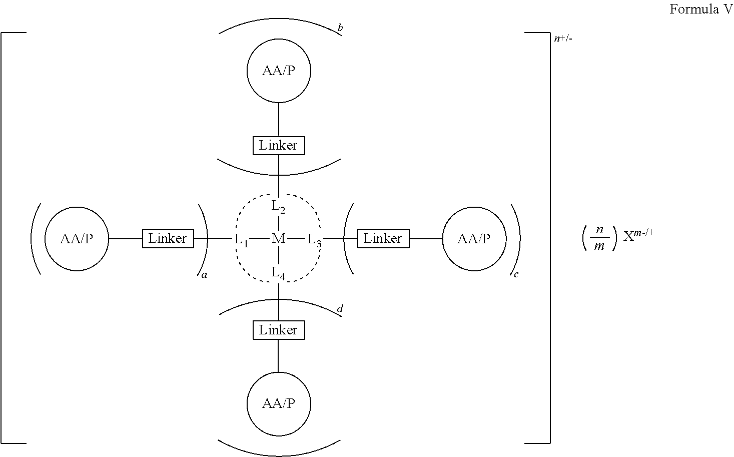

Wherein:
   (a) M is a metal center selected from Pt(II), Pd(II), Ni(II), Ir(I), Rh(I), Au(III), Ag(III), and Cu(III);
   (b) L₁, L₂, L₃, and L₄ represent the one or more coordinating ligands, wherein each ligand can offer at least one donor atom for coordination to the metal center;
   (c) n+/− is a charged state of the compound, wherein n is zero or a positive integer, such as 1, 2, 3, 4, and 5;
   (d) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
   (e) m−/+ is a charged state of the counterion, wherein m is zero or a positive integer, such as 1, 2, 3, 4, and 5, wherein m=n or m≠n;
   (f) n/m represents a stoichiometry of the counterions in Formula V;
   (g) the four dashed lines represent an optional independent covalent linking between two ligands, an optional independent fusion of ring moieties from two ligands, or a combination thereof,
   (h) L₁, L₂, L₃, and L₄ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻, RO⁻, RS⁻, RSe⁻, N═N═N—R, N≡C—R wherein N is a donor atom, C≡N—R wherein C is a donor atom, NR¹R²R³, PR¹R²R³, and AsR¹R²R³. (R, R¹, R², and R³ would be defined above). For example, R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
   (i) a, b, c, d are independently 0 or a positive integer, such as 1, and a+b+c+d>0;
   (j) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and combination thereof;
   (k) AA are selected from amino acids, their derivatives, or combination of amino acids and/or their derivatives, preferably AA are selected from:

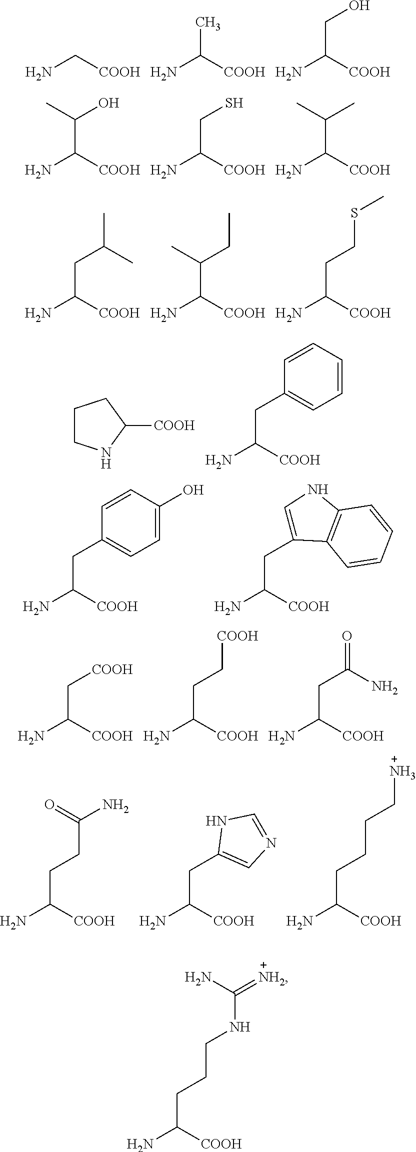

and
   (l) P represents positive charge-containing structure(s), preferably substituted and unsubstituted amine, ammonium, pyridinium, pyrrodinium, phosphonium, imidazolium, sulfonium, and their derivatives.
9. The compound of any of the preceding paragraphs, wherein the compound has a structure of Formula VI:

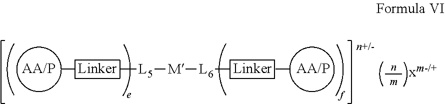

Wherein:
   (a) M′ is a metal center selected from Ni(0), Pd(0), Pt(0), Cu(I), Ag(I), Au(I), Zn(II), Cd(II) and Hg(II);
   (b) L₅ and L₆ represent the one or more coordinating ligands, wherein each ligand can offer at least one donor atom for coordination to the metal center; and
   (c) e and f are independently 0 or a positive integer, such as 1, and e+f>0.
10. The compound of any of the preceding paragraphs, wherein the compound has a structure of Formula VII:

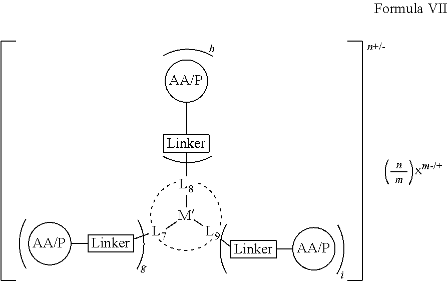

Wherein:
   (a) L₇, L₈, and L₉ represent the one or more coordinating ligands, wherein each ligand can offer at least one donor atom for coordination to the metal center;
   (b) g, h and i are independently 0 or a positive integer, such as 1, and g+h+i>0.
11. The compound of any of the preceding paragraphs, wherein the metal complex binds to the analyte via noncovalent interactions, wherein the noncovalent interactions comprise electrostatic interactions, hydrogen bonding interactions, hydrophobic interactions, or combinations thereof.
12. The compound of any of the preceding paragraphs, wherein the metal complex has a planar structure or a partially planar structure.
13. The compound of any of the preceding paragraphs, wherein the aggregation and supramolecular self-assembly of the metal complex creates one or more changes in the photophysical properties of the metal complex.
14. The compound of paragraph 13, wherein the one or more changes in the photophysical properties comprise a change in optical absorbance, luminescence, or combinations thereof.
15. The compound of paragraph 14, wherein the change in luminescence comprises an increase in the luminescence quantum yield and/or emission intensity, and/or a shift in emission energy or wavelength.
16. The compound of any of the preceding paragraphs, wherein the compound is selected from.

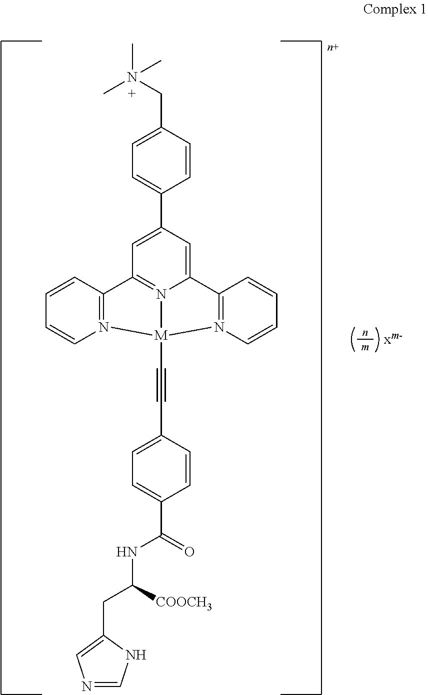

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

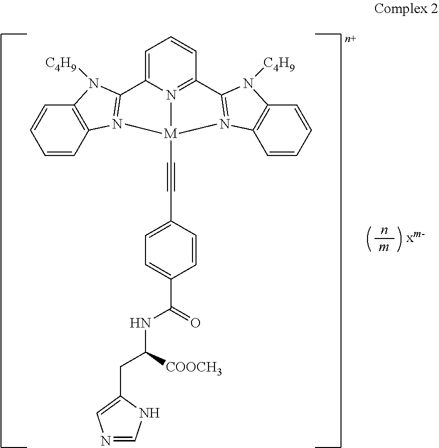

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

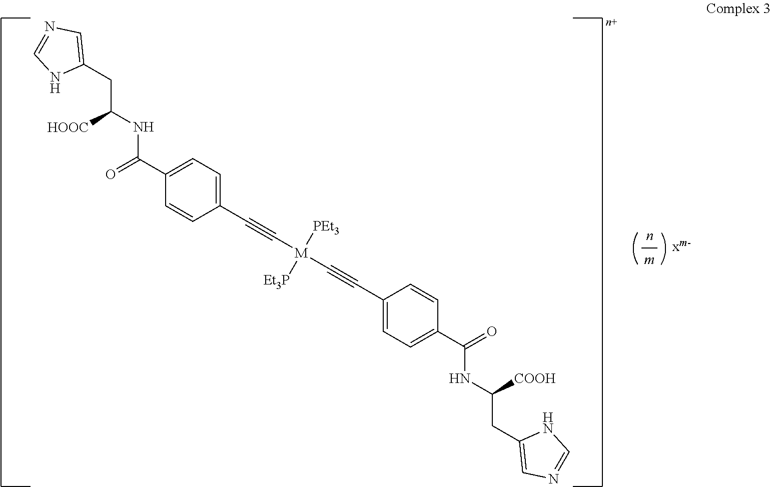

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

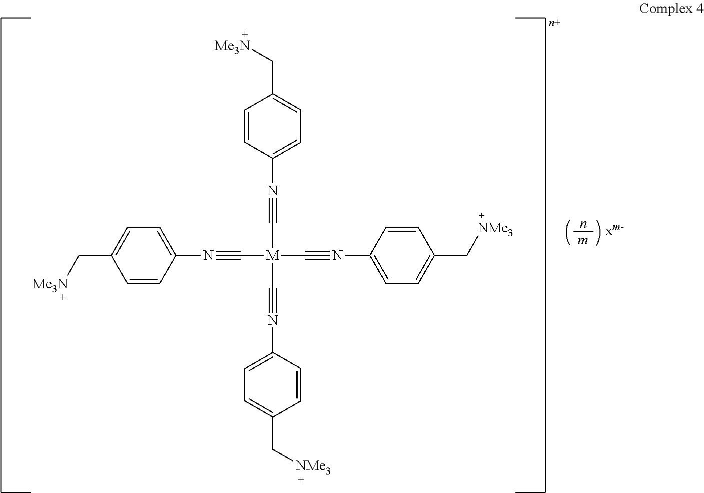

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

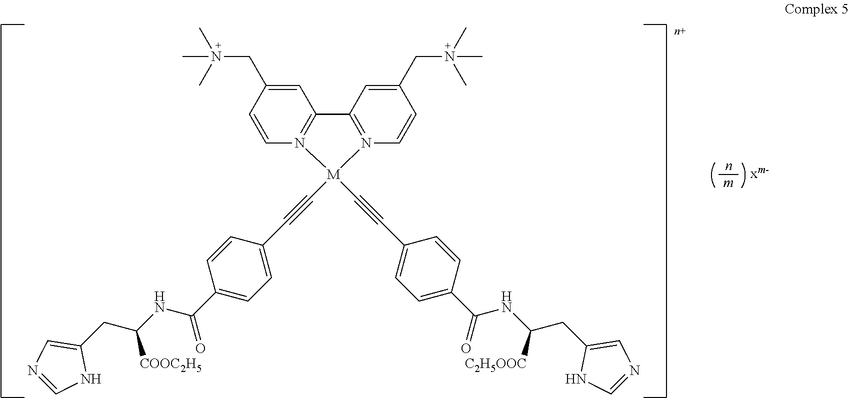

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

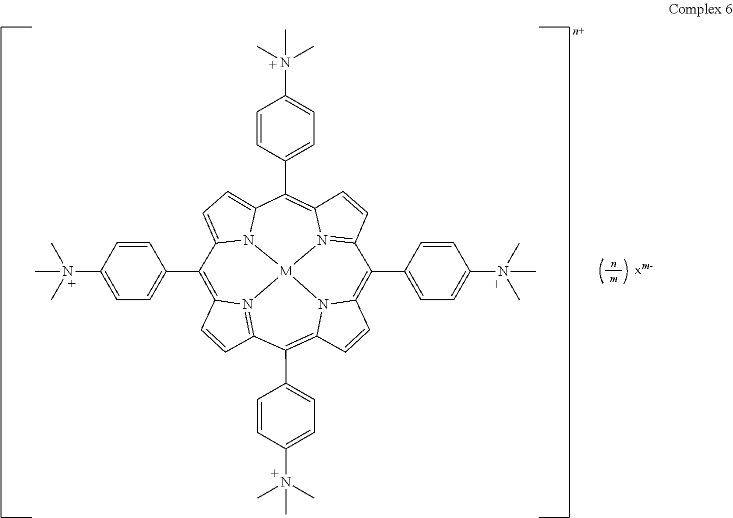

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

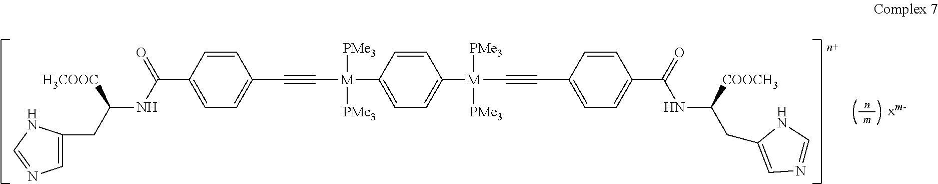

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

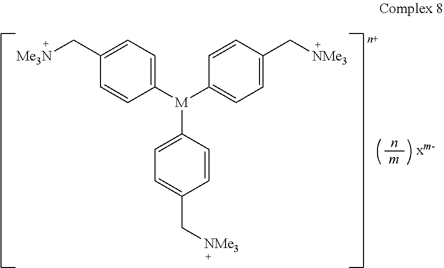

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

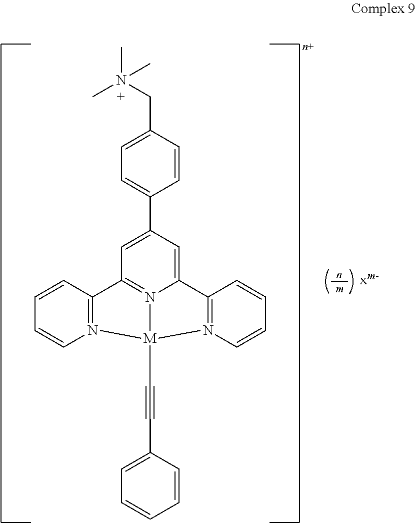

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

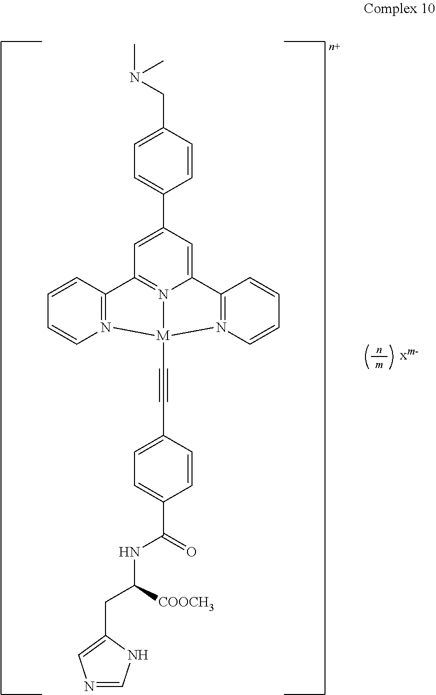

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula;

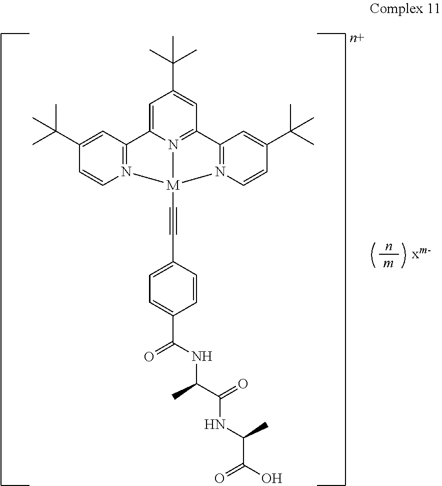

Wherein M stands for metal center, which can be selected from Ir(I), Rh(I), Pt(II), Pd(II), Ni(II), Au(III), Ag(III), or Cu(III). n represents the number of charges of the metal complex in the formula, wherein n is zero or a positive integer. X represents the counterion for charge neutrality, wherein n is zero or a positive integer, m=n or m≠n. n/m stands for the stoichiometry of the counterions in the formula.
17. The compound of any one of the preceding paragraphs, wherein the analyte can be glycans, such as sialic acids, polysialic acids, etc., or cancer cells.
18. A method for detecting an analyte in a sample (preferably a biological sample), the method involving: (a) combining the compound of any one of the preceding paragraphs with the sample, (b) detecting changes in the photophysical properties of the metal complex, wherein detection of changes in the photophysical properties of the metal complex indicates the presence of aggregation and supramolecular self-assembly of the metal complex, wherein the presence of aggregation and supramolecular self-assembly of the metal complex indicates the presence of the analyte in the sample.
19. The method of paragraph 18, wherein the analyte is selected from glycans, such as sialic acids, polysialic acids, etc., or cancer cells.
20. The method of paragraph 18 or paragraph 19, wherein the sample comprises a human or non-human animal bodily fluid, a human or non-human animal tissue, or a combination thereof.
21. A method for testing the efficacy of an inhibitor to remove sialic acids on the cellular surface, the method involving:
   (a) combining the compound of any one of the preceding paragraphs with an inhibitor-treated sample containing a protein or peptide and, separately, with a corresponding untreated sample containing the protein or peptide; and
   (b) comparing the photophysical properties of the metal complex between the inhibitor-treated sample and the corresponding untreated sample;
      wherein the magnitude of the difference in the photophysical properties of the metal complex between the two samples indicates the extent of change in the state of aggregation and supramolecular self-assembly of the metal complex, wherein the extent of change in the state of aggregation and supramolecular self-assembly of the metal complex indicates the efficacy of the inhibitor.
22. A method for imaging an analyte in a sample, the method comprising:
   (a) combining the compound of any one of the preceding paragraphs with the sample under conditions to allow for binding of the metal complex of the compound with the analyte and subsequent aggregation and supramolecular self-assembly of the metal complex, wherein aggregation and supramolecular self-assembly of the metal complex generates changes in the photophysical properties of the metal complex; and
   (b) imaging the analyte based on one or more photophysical properties that are specific for the metal complex after aggregation and supramolecular self-assembly.
23. The method of paragraph 22, wherein the analyte is selected from glycans, such as sialic acids, polysialic acids, etc., or cancer cells.
24. The method of paragraph 22 or paragraph 23, wherein the sample contains eukaryotic cells optionally selected from the groups consisting of 3T3 cells, A549 cells, Chinese hamster ovary (CHO) cells, HEK293 cells, HeLa cells, HepG2 cells, and HT1080 cells.
25. A kit containing, in one or more containers, one or more compounds of any one of the preceding paragraphs and optionally instructions for use, preferably wherein the kit is for use in detecting and/or imaging an analyte.
26. The kit of paragraph 25, wherein the analyte is selected from glycans, such as sialic acids, polysialic acids, etc., or cancer cells.
27. The kit of paragraph 25 or paragraph 26, further comprising a carrier.
28. The kit of any one of the preceding paragraphs, wherein the presence of the analyte induces aggregation and supramolecular self-assembly of the metal complex thereon after binding, wherein the aggregation and supramolecular self-assembly of the metal complex can be detected by changes in the photophysical properties of the metal complex.

The methods, compounds, and compositions herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. All parts or amounts, unless otherwise specified, are by weight.

### EXAMPLES

### Example 1: Synthesis and Characterization of Complex 1-Pt

### Materials and Methods

Complex 1-Pt was prepared by stirring [Pt{tpy-(C₆H₄CH₂NMe₃-4)-4′}Cl](PF₆)₂ (0.12 g, 0.17 mmol) triethylamine (1 mL), ligand HHIS (0.15 g, 0.51 mmol) and CuI (catalytic amount) in degassed DMF (6 mL) under nitrogen at room temperature. The obtained precipitation after pouring mixture into diethyl ether was collected by centrifuge and subsequent recrystallization by diffusion of diethyl ether vapor into the methanol-acetonitrile solution of the solid gave a dark-brown solid. The final water-soluble complex was then prepared by salt metathesis reaction with LiCl. The product was obtained as a dark-brown solid.

Proton nuclear magnetic resonance (¹H NMR) spectra were recorded on a Bruker AVANCE 500 Fourier Transform NMR Spectrometer (500 MHz) with tetramethylsilane as an internal standard. Negative fast atom bombardment (FAB) mass spectra were recorded on a Thermo Fisher Scientific DFS High Resolution Magnetic Sector Mass Spectrometer. Elemental analyses were carried out on a Thermo Fisher Scientific Flash EA 1112 Elemental Analyzer at the Institute of Chemistry, Chinese Academy of Sciences.

### Results

### (i) Characterization of Complex 1-Pt

Yield: 0.036 g (35%). ¹H NMR (500 MHz, DMSO-d₆, 298 K, δ/ppm) δ 9.27 (d, J=9.3 Hz, 2H, terpyridine H), 9.14 (s, 2H, terpyridine H), 8.92 (d, J=8.9 Hz, 2H, terpyridine H), 8.92 (m, 1H, imidazole H), 8.63 (t, J=8.6 Hz, 2H, terpyridine H), 8.36 (d, J=8.4 Hz, 2H, phenyl H), 8.01 (t, 2H, J=8.0 Hz, terpyridine H), 7.87 (m, 4H, phenyl H), 7.87 (m, 1H, imidazole H), 7.65 (m, 2H, phenyl H), 4.65 (s, 1H, —CH(NH)—), 4.65 (s, 2H, —CH₂N⁺(CH₃)₃), 3.63 (s, 3H, —CH₃), 3.11 (s, 9H, —CH₂N⁺(CH₃)₃). HRMS (positive-ion ESI) cald. For C₄₁H₃₉N₇O₃Pt m=436.1377; found: 436.1368 [M−2Cl]⁺. Elemental analysis calcd. (%) for C₄₁H₃₉N₇O₃Cl₂Pt·2CH₂Cl₂·H₂O: C, 45.60; H, 3.98; N, 8.66. found: C, 45.13, H, 4.12, N, 8.69.

### Example 2: Photophysical Properties of Complex 1-Pt

### Materials and Methods

The photophysical properties of complex 1-Pt were measured at a concentration of 30 μM.

### Results

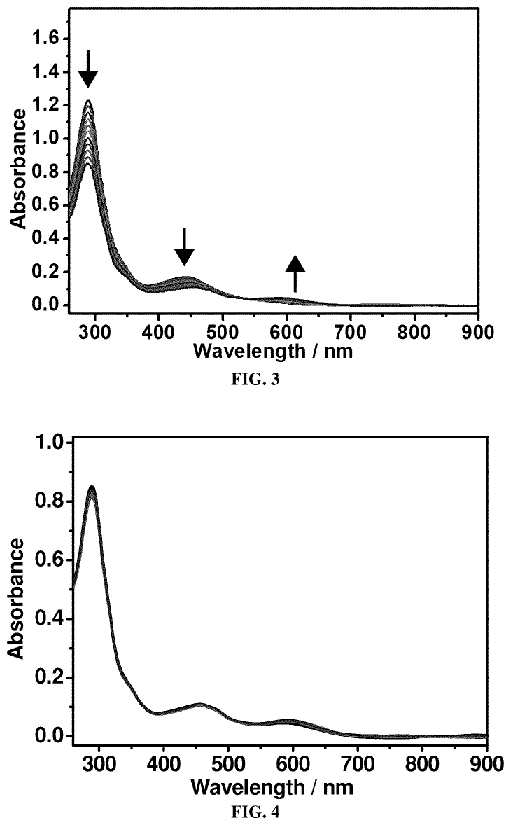

UV-Vis spectra of complex 1-Pt in aqueous solution at 298K displayed high-energy absorption bands at 260-290 nm and low-energy bands at 440-470 nm (FIG. 3). The high-energy absorption bands have been ascribed to intraligand π→π* transitions of terpyridine and alkynyl ligand.

Meanwhile, the lower-energy absorption bands have been assigned as an admixture of metal-to-ligand charge transfer (MLCT) [dπ(Pt)→π*(tpy)] and ligand-to-ligand charge transfer (LLCT) [π(C≡C)→π*(tpy)] transitions

The occurrence of the absorption shoulder band at 600 nm upon addition of polysialic acid which could be ascribed to the metal-metal-to-ligand charge transfer (MMLCT) transition demonstrated the self-assembly of the metal complex. The emission band at 760 nm was typical of the triplet metal-metal-to-ligand charge transfer (³MMLCT) excited state.

### Example 3: Detection for Polysialic Acids Via UV-Vis and Emission Spectroscopy

### Materials and Methods

Different concentrations (0-90 μM) of polysialic acids were added to a solution of complex 1-Pt (30 μM) in Tris-HCl buffer (10 mM Tris, 10 mM NaCl, pH=8.0). UV-Vis absorption spectra and emission spectra were recorded with the increasing concentration of polysialic acids at 25° C. The emission spectra were recorded at an excitation wavelength of 530 nm.

### Results

FIG. 3 demonstrated UV-vis spectra of complex 1-Pt and the gradual changes upon addition of increasing concentration of polysialic acids (0-114 M). Addition of polysialic acids (0-2 equiv.) to an aqueous buffer solution of complex1-Pt gave rise to the decline and a slight red shift of the low-energy band at 445 nm and the apparent growth of the lower-energy absorption shoulder band at 600 nm, which was due to supramolecular self-assembly and aggregation of the metal complex.

FIG. 4 demonstrated that the subsequent addition of polysialic acids (2-3.8 equiv.) made no difference to the absorption band of complex 1-Pt, suggesting that the binding might have reached an equilibrium.

FIG. 5 showed the emission spectra of complex 1-Pt and the gradual changes upon addition of increasing concentration of polysialic acids (0-90 μM). Addition of polysialic acids (0-3 equiv.) to an aqueous buffer solution of complex1-Pt gave rise to a luminescence turn-on at 760 nm, which was due to supramolecular self-assembly and aggregation of the metal complex.

FIG. 6 exhibited a clear increase of the emission band at 760 nm. The remarkable enhancement of the emission band suggested the formation of aggregates.

### Example 4: High Binding Affinity of Complex 1-Pt Toward Polysialic Acids

### Materials and Methods

To a solution of complex 1-Pt in Tris-HCl buffer (10 mM Tris, 10 mM NaCl, pH=8.0), different concentrations of polysialic acids were added. Emission spectra were recorded at 25° C. at an excitation wavelength of 530 nm. The emission intensity at 800 nm was fit using the Hill equation ((see similar examples of data fitting in Donabedian et al., ACS Chem. Neurosci., 6, 1526-1535 (2015)); Goutelle et al., Fundam. Clin. Pharmacol., 22, 633-648 (2008); and Gesztelyi et al., Arch. Hist. Exact Sci., 66, 427-438 (2012))

$$y = \frac{{x}^{n}}{{K}_{d} + {x}^{n}}$$

### Wherein:

y stands for the corrected emission intensity; x stands for the concentration of polysialic acids; n stands for the Hill coefficient, which can be used to indicate the cooperativity of binding to polysialic acids; K_{d} stands for the apparent dissociation constant.

### Results

The apparent dissociation constant between complex 1-Pt and polysialic acids has been calculated as 9.52×10⁻⁹ M, demonstrating its high affinity toward polysialic acid.

### Example 5. Differentiation of Polysialic Acid from Sialic Acid by Complex 1-Pt

### Materials and Methods

Different concentrations (0-90 μM) of sialic acids (Neu5Ac) were added to a solution of complex 1-Pt (30 μM) in aqueous solution. Emission spectra were recorded with the increasing concentration of sialic acid at 25° C. The emission spectra were recorded at an excitation wavelength of 530 nm.

### Results

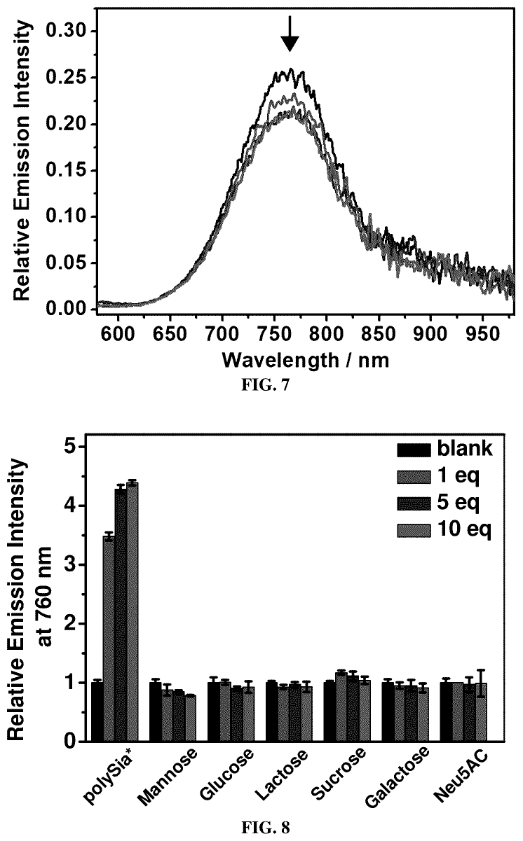

FIG. 7 showed the emission spectra of complex 1-Pt and the gradual changes upon addition of increasing concentrations of sialic acid (0-90 μM). Addition of Neu5Ac (0-3 equiv.) to an aqueous buffer solution of complex1-Pt gave rise to a decreasing luminescence at 760 nm, which was due to disassembly of the metal complex. The results showed that different responding behavior of complex 1-Pt toward polysialic acid and sialic acid can induce different changes of luminescence.

### Example 6. High Selectivity of Complex 1-Pt Toward Polysialic Acid Over Other Monosaccharides

### Materials and Methods

Different monosaccharides (1, 5, 10 equiv. of mannose, glucose, lactose, sucrose, galactose) were individually mixed with complex 1-Pt (30 μM) in Tris-HCl buffer (10 mM Tris, 10 mM NaCl, pH=8.0). Emission spectra were recorded at 25° C. The emission spectra were recorded at an excitation wavelength of 530 nm.

### Results

FIG. 8 showed that the emission spectra of complex 1-Pt upon addition of different monosaccharides (1, 5, 10 equiv. of mannose, glucose, lactose, sucrose, galactose) had been measured to study the selectivity of complex 1-Pt. The results showed that complex 1-Pt showed no clear spectroscopic response toward other monosaccharides, demonstrating that the presence of other monosaccharides would not affect the sensing ability of complex 1-Pt toward polysialic acids.

### Example 7. Complex 1-Pt, Complex 9-Pt, and Complex 10-Pt can be Used to Visualize Sialic Acids on the Surface of Cancer Cells

### Materials and Methods

HepG2 cells were cultured with DMEM culture medium supplemented with 10% FBS in a humidified incubator at 37° C. where the level of CO₂ was kept constant at 5%. HeLa cells were cultured with DMEM culture medium supplemented with 10% FBS in a humidified incubator at 37° C. where the level of CO₂ was kept constant at 5%. Then the cells were adhered onto a sterile coverslip in a 35-mm cell culture dish and then were cultured for 48 hours in a humidified incubator. After incubation, the cells were washed 3 times using PBS buffer.

In the time-dependent cell imaging experiments, complex 1-Pt (10 μM) was added to the cell dish and the cells were incubated in an incubator for different times (0.5 h, 1 h, 2 h, 6 h, 8 h). Confocal imaging assays were conducted on a Leica TCS SPE Confocal Scanning Microscope. The confocal images were obtained at an excitation wavelength of 405 nm and the emission was collected at 700-800 nm, using the 63× oil objective.

In the concentration-dependent cell imaging experiments, complex 1-Pt (10 μM, 20 μM, 30 μM) was added to the cell dish and the cells were incubated in an incubator for 0.5 h. Confocal imaging assays were conducted on a Leica TCS SPE Confocal Scanning Microscope. The confocal images were obtained at an excitation wavelength of 405 nm and the emission was collected at 700-800 nm, using the 63× oil objective.

### Results

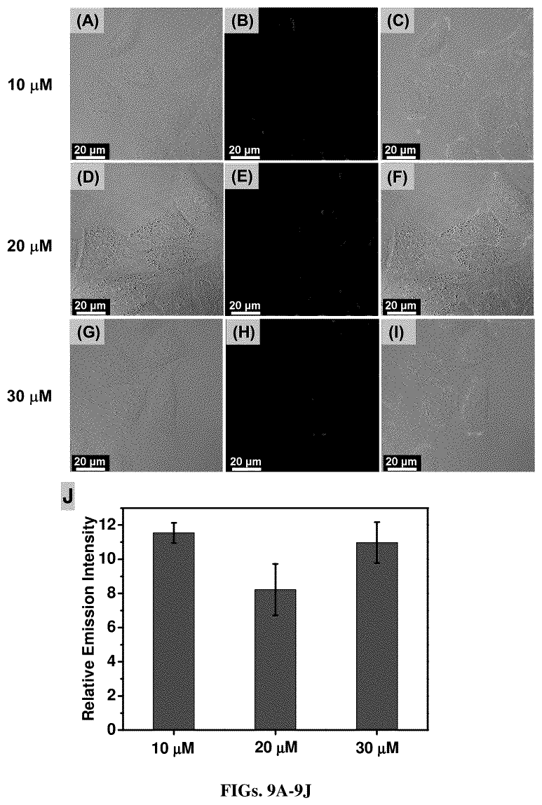

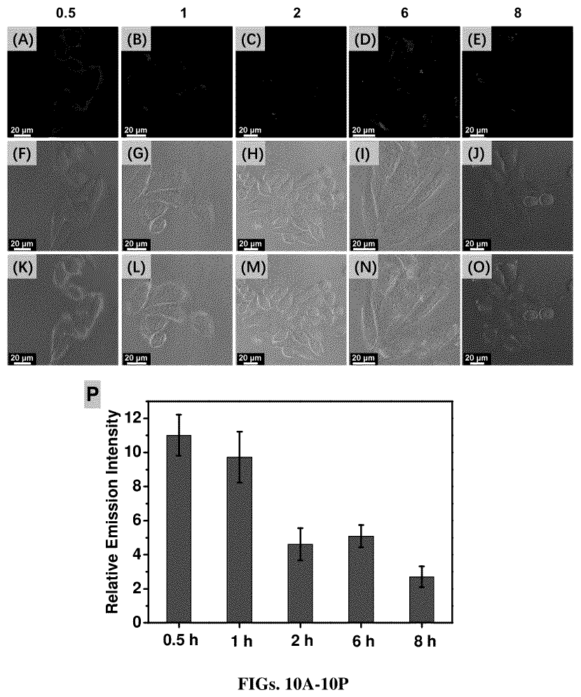

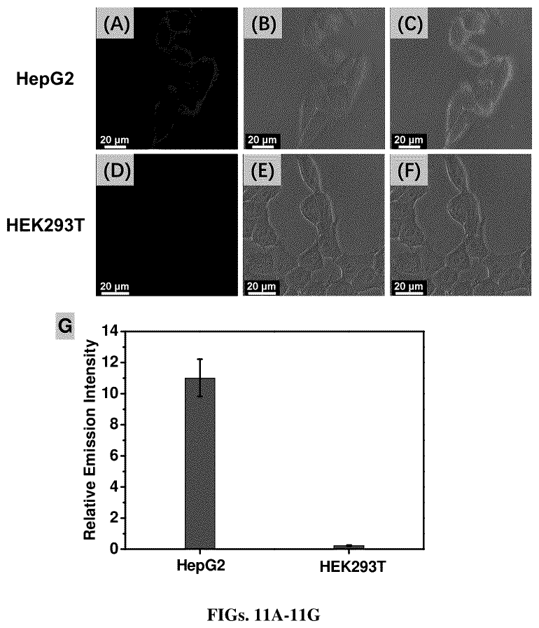

FIGS. 10A-10P showed time-dependent cell imaging experiments of complex 1-Pt. The accumulation of complex 1-Pt can be observed on the surface of cancer cells within 0.5 h.

FIGS. 9A-9J showed concentration-dependent cell imaging experiments of complex 1-Pt. The luminescent signals of cells incubated with complex 1-Pt with different concentrations (10 μM, 20 μM, 30 μM) were similar, so 10 μM was chosen as the working concentration for better biological compatibility.

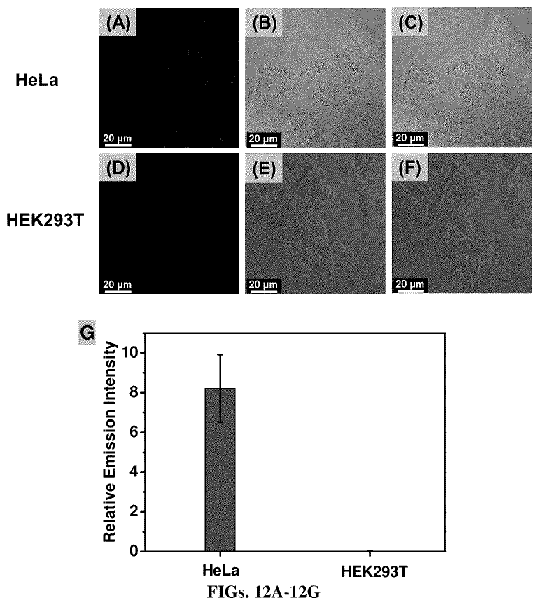

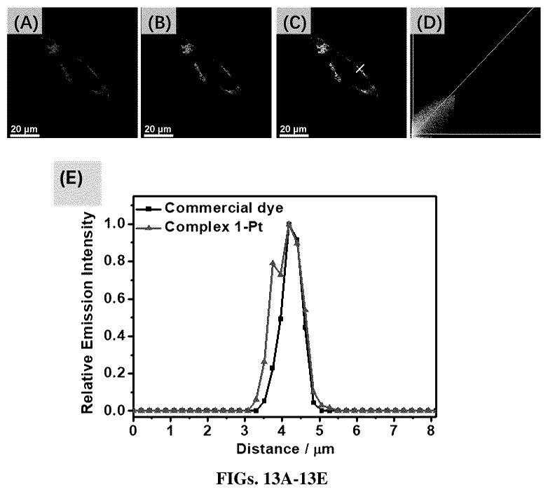

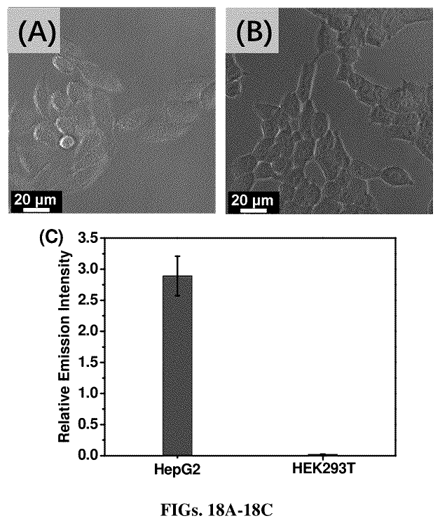

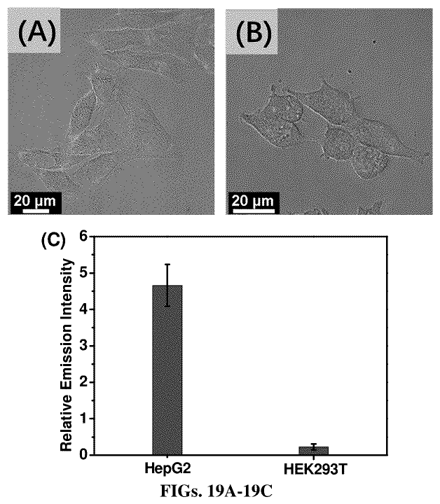

FIGS. 11A-11C and 12A-12C show that upon incubation with complex 1-Pt at 37° C. for 0.5 h, the bright luminescence signal can be observed on the surface of HepG2 cells and HeLa cells. FIG. 18A and FIG. 19A show that upon incubation with complex 9-Pt and complex 10-Pt, the luminescence signal with weaker relative emission intensity can also be observed on the surface of HepG2 cells.

### Example 8. Differentiation of Cancer Cells from Normal Cells by Complex 1-Pt, Complex 9-Pt and Complex 10-Pt

### Materials and Methods

HepG2 cells were cultured with DMEM culture medium supplemented with 10% FBS in a humidified incubator at 37° C. where the level of CO₂ was kept constant at 5%. HEK293T cells were cultured with DMEM culture medium supplemented with 10% FBS in a humidified incubator at 37° C. where the level of CO₂ was kept constant at 5%. Then the cells were adhered onto a sterile coverslip in a 35-mm cell culture dish and then were cultured for 48 hours in a humidified incubator. Then complex 1-Pt was added to the cell dish and the cells were incubated in an incubator for 0.5 h. Confocal imaging assays were conducted on a Leica TCS SPE Confocal Scanning Microscope. The confocal images were obtained at an excitation wavelength of 405 nm and the emission was collected at 700-800 nm, using the 63× oil objective.

FITC-conjugated lectin was used to stain sialic acids on the surface of cancer cells and normal cells. The confocal images were obtained using the 63× oil objective. Luminescence confocal images were obtained at an excitation wavelength of 405 nm and the emission was collected at 700-800 nm for complex 1-Pt while at an excitation wavelength of 488 nm and the emission was collected at 500-550 nm for FITC-conjugated lectins.

### Results

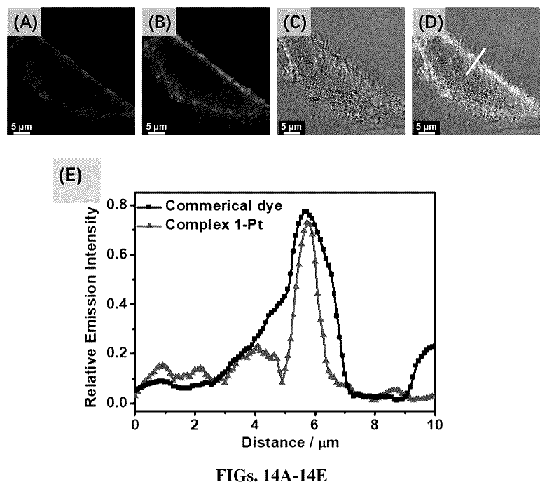

FIGS. 14A-14E show that complex 1-Pt shows intense luminescence signals while FITC-conjugated lectin also exhibits intense fluorescence signals on the surface of live HepG2 cells. Besides, the colocalization coefficient between complex 1-Pt and FITC-conjugated lectin is 0.87, confirming the staining ability of complex 1-Pt toward sialic acids on the cellular surface. However, complex 1-Pt, complex 9-Pt, and complex 10-Pt show no signals on the surface of live HEK293T cells (FIGS. 11D-11F, 12D-12F, 18B, and 19B). The bar graphs show that complex 1-Pt exhibits distinct emission signals on the surface of cancer cell (HeLa cells and HepG2 cells) and normal cells (HEK293T cells) while complex 9-Pt and complex 10-Pt exhibit less apparent contrast signals (FIGS. 11G, 12G, 18C, and 19C) The results indicate that complex 1-Pt, complex 9-Pt, and complex 10-Pt can differentiate cancer cells from normal cells by distinct luminescence signals, avoiding the possibility of a false-positive result. Complex 1-Pt exhibits the best differentiation ability among the three complexes.

### Example 9. Staining of Cellular Membrane by Complex 1-Pt

### Materials and Methods

HepG2 cells were cultured with DMEM culture medium supplemented with 10% FBS in a humidified incubator at 37° C. where the level of CO₂ was kept constant at 5%. Then the cells were adhered onto a sterile coverslip in a 35-mm cell culture dish, and then were cultured for 48 hours in a humidified incubator. Then complex 1-Pt (10 μM) was added to the cell dish and the cells were incubated in an incubator for 0.5 h. And Deep Red Plasma membrane stain tracker (5 μg/mL) was used as a commercialized staining dye for cellular membrane. The cell Confocal imaging assays were conducted on a Leica TCS SPE Confocal Scanning Microscope. The confocal images were obtained using the 63× oil objective. Luminescence confocal images were obtained at an excitation wavelength of 405 nm and the emission was collected at 700-800 nm for complex 1-Pt while at an excitation wavelength of 635 nm and the emission was collected at 650-670 nm for Deep Red Plasma membrane stain tracker.

### Results

The colocalization assays with a commercialized membrane-tracker had been studied using HepG2 cells to confirm the ability of complex 1-Pt staining cellular membrane. FIGS. 14A-14E indicated that the membranes of HepG2 cells were strongly co-stained with complex 1-Pt and membrane tracker.

### Example 10. Visualization of the Variation in the Level of Sialic Acids after the Treatment of Neuraminidase by Complex 1-Pt

### Materials and Methods

HepG2 cells were cultured with DMEM culture medium supplemented with 10% FBS in a humidified incubator at 37° C. where the level of CO₂ was kept constant at 5%. Then the cells were adhered onto a sterile coverslip in a 35-mm cell culture dish, and then were cultured for 48 hours in a humidified incubator. Then a solution of neuraminidase (0.1 U/mL) was used to induce a decreasing level of sialic acids. Then complex 1-Pt (10 μM) was added to the cell dish and the cells were incubated in an incubator for 0.5 h. The cell Confocal imaging assays were conducted on a Leica TCS SPE Confocal Scanning Microscope. The confocal images were obtained using the 63× oil objective. Luminescence confocal images were obtained at an excitation wavelength of 405 nm and the emission was collected at 700-800 nm for complex 1-Pt.

### Results

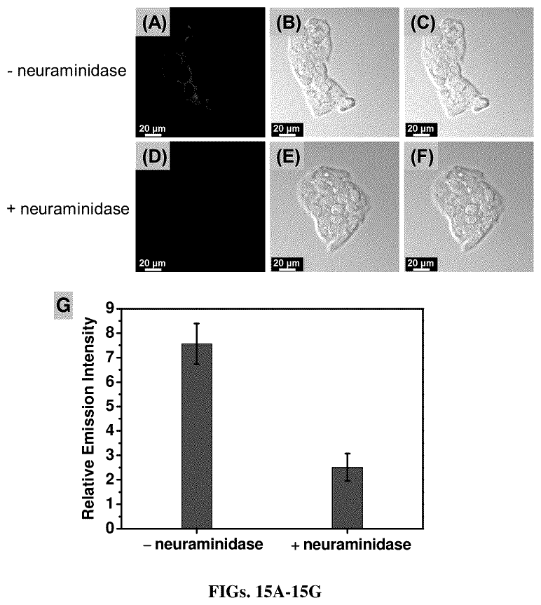

FIGS. 15A-15G showed that complex 1-Pt shows intense luminescence signals on the surface of live HepG2 cells without any treatments while no luminescence signals were observed in the live HepG2 cells after treatment with neuraminidase (0.1 U/mL). The result suggested that complex 1-Pt can visualize the variation in the luminescence signals of sialic acids on the cellular surface, which can be used to screen the inhibitors for removing sialic acids.

### Example 11. Low Cytotoxicity of Complex 1-Pt

### Materials and Methods

HepG2 cells were adhered onto a 96-well plate. Each well has about 10,000 cells using Dulbecco's Modified Eagle medium (DMEM) supplemented with 10% fetal bovine serum (FBS) (100 μL) as incubation medium and the 96-well plate was put in a humidified incubator (CO₂ level: 5%) at 37° C. Different concentrations of complex 1-Pt (0.78, 1.56, 3.13, 6.25, 12.5, 25, 50, 100 μM) were added and the cells were incubated at 37° C. for 24 hours. After incubation, 10 μL of 3-(4,5-dimethyl-2-thiazolyl)-2,5-diphenyl-2H-tetrazolium bromide (MTT) solution (5 mg/mL) was added to each well and the plate was incubated at 37° C. for 3 hours. After the solution was removed, DMSO (200 μL) was added to dissolve the precipitated formazan. Then the absorbance of formazan at 570 nm was measured with a microplate absorbance reader. The cell viability was expressed as a percentage ratio of the absorbance of the cells treated with complex 1-Pt to that of the controls.

### Results

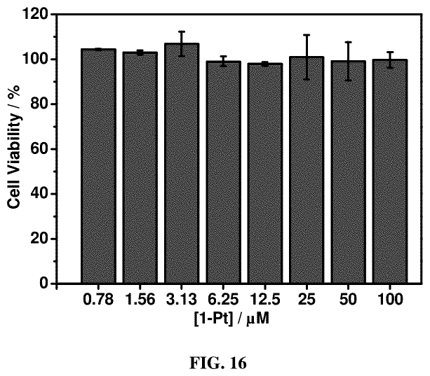

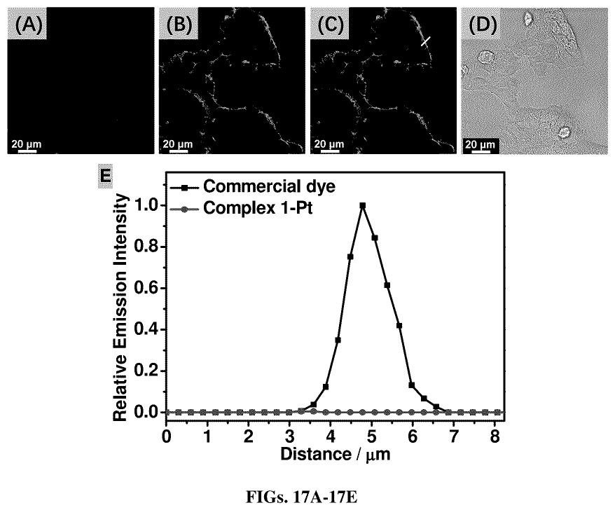

FIG. 16 showed MTT cell viability assay for 24 h to study the cytotoxicity of complex 1-Pt toward HepG2 cells. The result showed that complex 1-Pt shows good biocompatibility and little cytotoxicity.

### Example 12. Differentiation of Cancer Cells from Normal Cells by Complex 1-Pt in Comparison Against Commercial Dyes

### Materials and Methods

Live HEK293T cells were stained with complex 1-Pt (10 μM) for 0.5 h, followed by incubation with paraformaldehyde fix solution for 15 mins and FITC-conjugated lectins (20 μg/mL) for 1 h.

### Results

The FIGS. 17A-FIG. 17D show the confocal images of live HEK293T cells as a normal cell line incubated with complex 1-Pt and FITC-conjugated lectin. It is found that the luminescence signal of complex 1-Pt is not observed in this normal cell as viewed in the confocal image with emission collected at 700-800 nm in FIG. 17A, while the FITC-conjugated lectin is found to be localized on the cell membrane of this normal cell line in the confocal image with emission collected at 500-550 nm in FIG. 17B.

This result and the results from FIGS. 13A-13E, where both complex 1-Pt and FITC-conjugated lectin show similar localization in HepG2 cells as a cancer cell line in the confocal images, indicate the ability of complex 1-Pt to stain cancer cells only and thus differentiate cancer cells from normal cells.

In clinical use, common probes based on traditional sensing methods are used, such as indocyanine green (ICG) and 5-aminolevulinic acid (5-ALA). The cancer cells are differentiated by the difference in intensity of the fluorescence signals, which suffers from strong background signals that interferes with detection accuracy. Since normal cells will not be stained by 1-Pt, this disclosed strategy accurately detects cancer cells with low false positive rate in comparison to the current commercial probes.

Regarding the kits for detection of sialic acids, the Sialic Acid Quantitation Kit by Sigma-Aldrich can only measure total sialic acid content from the amount of N-acetylneuraminic acid, either free, or in glycoproteins, cell surface glycoproteins, polysialic acids and capsular polysaccharides, while the disclosed compound(s) can more selectively detect polysialic acid or other sialic acid polymers as viewed from the UV-vis and emission spectra provided in the data in instant disclosure. Therefore, the assay is superior over the commercially available kit. In sum, a significant quantity of sialic acids is present on the surface of cancer cells which ranges in the orders of 10⁷ to 10¹¹ molecules per cell, facilitating their adherence to vascular endothelium and assisting in evading immune system recognition (Narayanan, et al., Ann. Clin. Lab Sci., 1994, 24, 376-384). The malignant or metastatic phenotypes of various cancers could result in distinct amounts of sialic acids on the cell surface. For example, a SMMIC-7721 cell exhibits approximately 1.37×10¹¹ sialic acids molecules; (Wang, et al., Anal. Chem., 2017, 89, 538-543); while a HeLa cells exhibits approximately 4.6×10⁷ sialic acids molecules (Xu, et al., Talanta, 2020, 209, 120579). Unlike current clinical probes, such as ICG and 5-ALA, compounds disclosed herein do not exhibit strong background signals resulting from the high autofluorescence that interfere with detection accuracy. Further, compounds disclosed herein can specifically bind to desired analytes through non-bonded interactions (particularly electrostatic interactions and hydrogen bonding), thereby enhancing selectivity and sensitivity to analytes. In addition, changes in photophysical properties resulting from supramolecular self-assembly of complexes after binding to a high density of sialic acids on the surface of cancer cells facilitate tumor identification, early diagnosis, and surgical guidance.

The experimental results significantly demonstrate the advantages and uniqueness of compounds disclosed herein. Notably, in aqueous solutions, a clear enhancement in luminescence properties of metal complexes upon addition of polysialic acids was observed, in sharp contrast to a slight decline in luminescence properties upon addition of sialic acid monomers. These findings demonstrate the efficacy of compounds disclosed herein. In cell imaging assays, the distinct luminescence signals were observed on the surface of the cancer cells (HeLa cells, and HepG2 cells) and normal cells (HEK293T cells), indicating applicability for early diagnosis and providing guidance in tumor removal surgery. A comparison with a commercialized dye revealed successful staining on the cancer cells membrane and minimal background signal on the normal cell membrane.

Those skilled in the art will recognize or be able to ascertain using no more than routine experimentation, many equivalents to the specific embodiments of the invention described herein. Such equivalents are intended to be encompassed by the following claims.

We claim:

1. A compound comprising one or more metal centers having d⁸ or d¹⁰ electronic configuration and one or more coordinating ligands comprising one or more donor atoms, wherein:
(a) the one or more metal centers have a coordination number of 2, 3, or 4, and are selected from Pt(II), Pd(II), Ni(II), Ir(I), Rh(I), Au(III), Ag(III), Cu(III), Ni(0), Pd(0), Pt(0), Cu(I), Ag(I), Au(I), Zn(II), Cd(II), Hg(II), and a combination thereof; and
(b) the one or more one donor atoms are selected from the group containing carbon (C), nitrogen (N), oxygen (O), phosphorus (P), sulfur (S), arsenic (As), and selenium (Se).

2. The compound of claim 1, having a chemical structure: 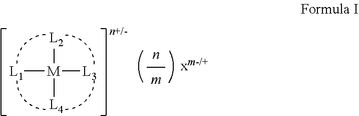
wherein: (a) M is a metal center selected from Pt(II), Pd(II), Ni(II), Ir(I), Rh(I), Au(III), Ag(III), and Cu(III), (b) L₁, L₂, L₃, and L₄ represent the one or more coordinating ligands, wherein each ligand can offer at least one donor atom for coordination to the metal center; (c) n+/− is a charged state of the compound, wherein n is zero or a positive integer, such as 1, 2, 3, 4, and 5; (d) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof; (e) m−/+ is a charged state of the counterion, wherein m is zero or a positive integer, such as 1, 2, 3, 4, and 5, wherein m=n or m≠n; (f) n/m represents a stoichiometry of the counterions in Formula I; and (g) the four dashed lines represent an optional independent covalent linking between two ligands, an optional independent fusion of ring moieties from two ligands, or a combination thereof.

3. The compound of claim 2, wherein L₁, L₂, L₃, and L₄ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻—, RO⁻—, RS⁻—, RSe⁻—, N═N═N—R, N—C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties).

4. The compound of claim 2, wherein the dashed lines stand for an optional linking between two ligands; or an optional fusion of rings from different ligands.

5. The compound of claim 1, having a chemical structure:
$${[ {L}_{5} — {M}^{′} — {L}_{6} ]}^{n + / -} ( \frac{n}{m} ) {X}^{m - / +} \qquad Formula II$$
wherein:
(a) M′ is a metal center selected from Ni(O), Pd(O), Pt(O), Cu(I), Ag(I), Au(I), Zn(II), Cd(II) and Hg(II); and
(b) L₅ and L₆ represent the one or more coordinating ligands, wherein each ligand can offer at least one donor atom for coordination to the metal center;
(c) L₅ and L₆ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻—, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(d) n+/− is a charged state of the compound, wherein n is zero or a positive integer, such as 1, 2, 3, 4, and 5;
(e) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
(f) m−/+ is a charged state of the counterion, wherein m is zero or a positive integer, such as 1, 2, 3, 4, and 5, wherein m=n or m≠n; and
(g) n/m represents a stoichiometry of the counterions in Formula II.

6. The compound of claim 1, having a chemical structure: 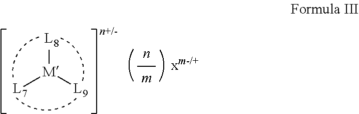
wherein:
(a) the compound exhibits a trigonal planar geometry, and M′ is a metal center selected from Ni(O), Pd(O), Pt(O), Cu(I), Ag(I), Au(I), Zn(II), Cd(II) and Hg(II);
(b) L₇, L₈, and L₉ represent the one or more coordinating ligands, independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻—, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(c) each ligand can offer at least one donor atom for coordination to the metal center;
(d) n+/− is a charged state of the compound, wherein n is zero or a positive integer, such as 1, 2, 3, 4, and 5;
(e) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄³⁻), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
(f) m−/+ is a charged state of the counterion, wherein m is zero or a positive integer, such as 1, 2, 3, 4, and 5, wherein m=n or m≠n; and
(g) n/m represents a stoichiometry of the counterions in Formula III.

7. The compound of claim 1, wherein at least one of the one or more coordinating ligands has a chemical structure: 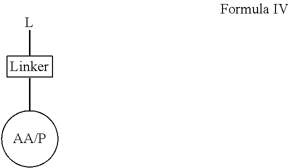
wherein:
(a) L stands for chemical moieties containing one or more donor atoms, preferably one donor atom, for coordination to the metal center of the metal complexes; preferably wherein the chemical moieties are selected from (i) five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene; and/or (ii) halide ions, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, C≡N⁻, CO wherein C is a donor atom, R—C≡C⁻, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(b) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and a combination thereof,
(c) AA are selected from amino acids, their derivatives, or combination amino acids and/or their derivatives, preferably AA are selected from: 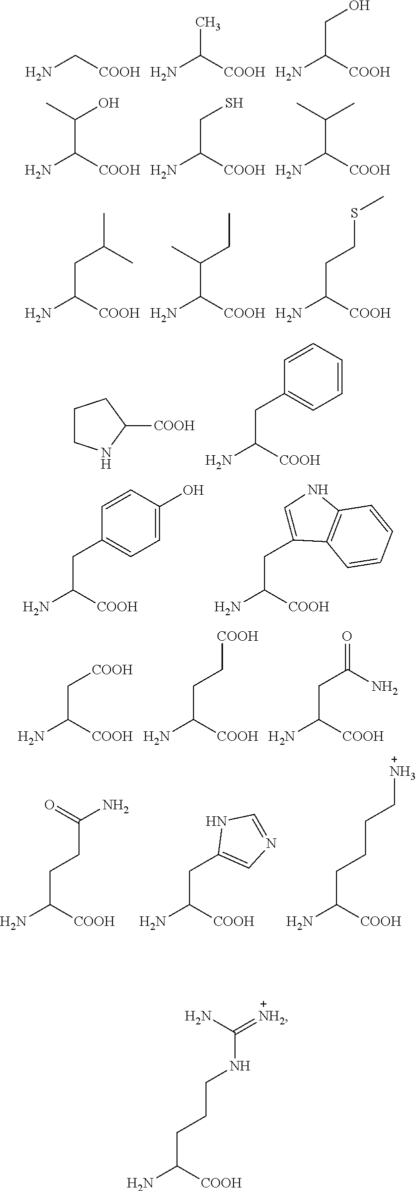
and
(d) P represents positive charge-containing structure(s), preferably substituted and unsubstituted amine, ammonium, pyridinium, pyrrodinium, phosphonium, imidazolium, sulfonium, and their derivatives.

8. The compound of claim 1, having a chemical structure: 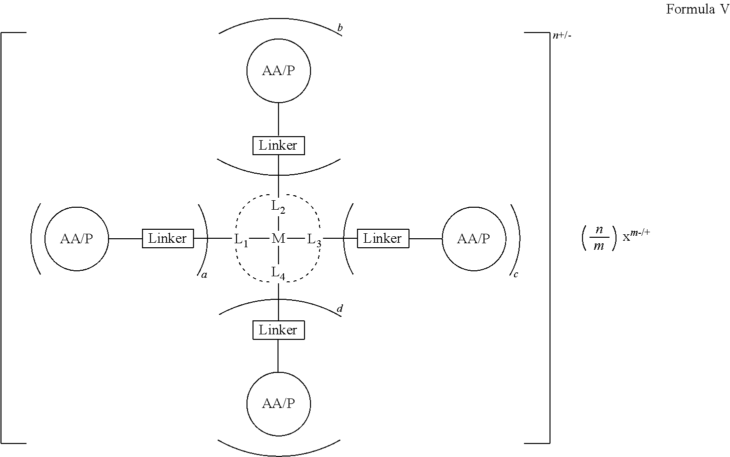
wherein:
(a) M is a metal center selected from Pt(II), Pd(II), Ni(II), Ir(I), Rh(I), Au(III), Ag(III), and Cu(III),
(b) L₁, L₂, L₃, and L₄ represent the one or more coordinating ligands, wherein each ligand can offer at least one donor atom for coordination to the metal center;
(c) n+/− is a charged state of the compound, wherein n is zero or a positive integer, such as 1, 2, 3, 4, and 5;
(d) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄⁻³), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
(e) m−/+ is a charged state of the counterion, wherein m is zero or a positive integer, such as 1, 2, 3, 4, and 5, wherein m=n or m≠n;
(f) n/m represents a stoichiometry of the counterions in Formula V;
(g) the four dashed lines represent an optional independent covalent linking between two ligands, an optional independent fusion of ring moieties from two ligands, or a combination thereof,
(h) L₁, L₂, L₃, and L₄ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C⁻—, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(i) a, b, c, d are independently 0 or a positive integer, such as 1, and a+b+c+d>0;
(j) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and combination thereof,
(k) AA are selected from amino acids, their derivatives, or combination of amino acids and/or their derivatives, preferably AA are selected from: 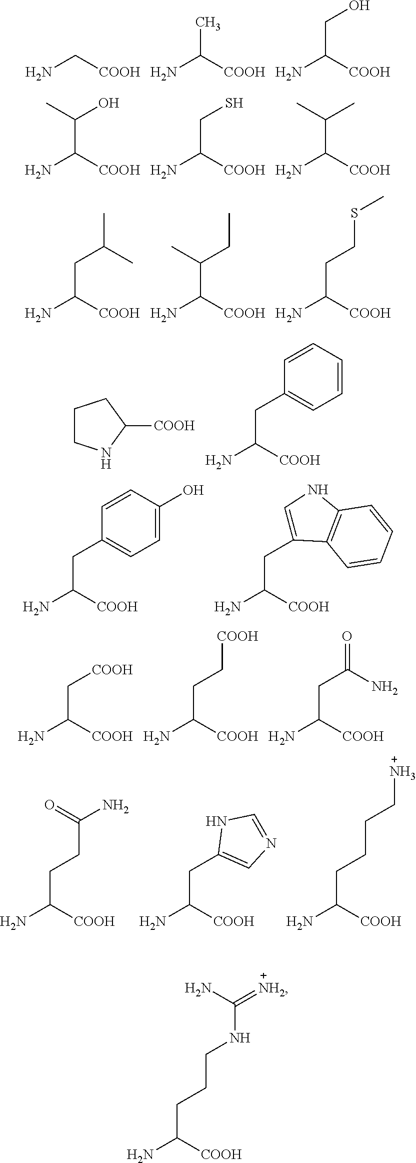
and
(l) P represents positive charge-containing structure(s), preferably substituted and unsubstituted amine, ammonium, pyridinium, pyrrodinium, phosphonium, imidazolium, sulfonium, and their derivatives.

9. The compound of claim 1, having a chemical structure: 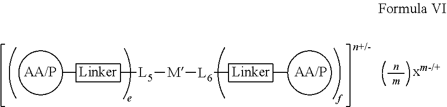
wherein:
(a) M′ is a metal center selected from Ni(O), Pd(O), Pt(O), Cu(I), Ag(I), Au(I), Zn(II), Cd(II) and Hg(II);
(b) L₅ and L₆ represent the one or more coordinating ligands, wherein each ligand can offer at least one donor atom for coordination to the metal center, and wherein L₅ and L₆ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O—NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C, RO⁻—, RS⁻—, RSe⁻—, N═N═N—R, N≡C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(c) e and f are independently 0 or a positive integer, such as 1, and e+f>0;
(d) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and a combination thereof,
(e) AA are selected from amino acids, their derivatives, or combination amino acids and/or their derivatives, preferably AA are selected from: 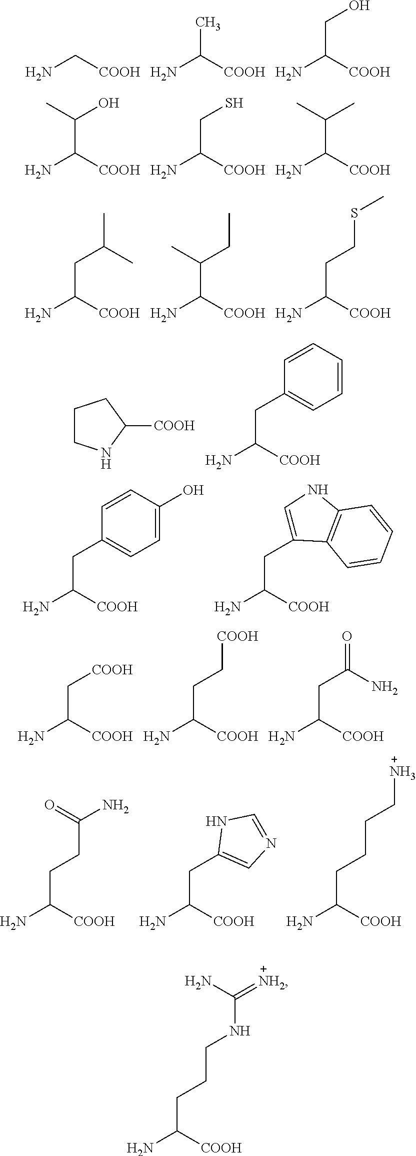
(f) P represents positive charge-containing structure(s), preferably substituted and unsubstituted amine, ammonium, pyridinium, pyrrodinium, phosphonium, imidazolium, sulfonium, and their derivatives;
(g) n+/− is a charged state of the compound, wherein n is zero or a positive integer, such as 1, 2, 3, 4, and 5;
(h) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄⁻³), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof;
(i) m−/+ is a charged state of the counterion, wherein m is zero or a positive integer, such as 1, 2, 3, 4, and 5, wherein m=n or m n; and
(j) n/m represents a stoichiometry of the counterions in Formula VI.

10. The compound of claim 1, having a chemical structure: 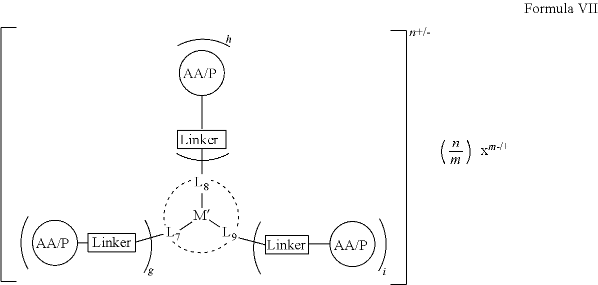
wherein:
(a) the compound exhibits a trigonal planar geometry, and M′ is a metal center selected from Ni(0), Pd(0), Pt(0), Cu(I), Ag(I), Au(I), Zn(II), Cd(II) and Hg(II);
(b) L₇, L₈, and L₉ represent the one or more coordinating ligands, wherein each ligand can offer at least one donor atom for coordination to the metal center, and wherein L₇, L₈, and L₉ are independently selected from C₆-C₅₀ arenes or C₃-C₅₀ heteroarenes, such as five-membered arenes and their derivatives, include but are not limited to, furan, pyrrole, thiophene, imidazole, pyrazole, oxazole, isoxazole, thiazole, benzofuran, isobenzofuran, indole, isoindole, benzothiophene, benzo[c]thiophene, benzimidazole, purine, indazole, benzisoxazole, benzothiazole; and/or six-membered arenes and their derivatives, include but are not limited to, benzene, pyridine, pyrazine, pyrimidine, pyridazine, 1,2,3-triazine, naphthalene, anthracene, quinoline, isoquinoline, quinoxaline, acridine, quinazoline, cinnoline, phthalazine, 1,2,4-triazine, 1,3,5-triazine, bipyridine, terpyridine, 2,6-bis(benzimidazol-2′-yl)pyridine, carbazole, dibenzothiophene, dibenzofuran, fluorene, halide, alkylamine, arylamine, alkylphosphine, arylphosphine, alkylarsine, arylarsine, SCN⁻ wherein S is a donor atom, O-NO₂⁻ wherein O is a donor atom, N₃⁻, O₂⁻, S₂⁻, H₂O, O—NO⁻ wherein O is a donor atom, NCS⁻ wherein N is a donor atom, NH₃, NO₂⁻ wherein N is a donor atom, N≡C⁻, CO wherein C is a donor atom, R—C≡C, RO⁻—, RS⁻—, RSe⁻—, N═N═N—R, N—C—R wherein N is a donor atom, C≡N—R wherein C is a donor atom, NR¹R²R³, PR¹R²R³, and AsR¹R²R³, wherein R, R¹, R², and R³ are independently selected from hydrogen, substituted or unsubstituted C₁-C₃₀ alkyl, C₂-C₃₀ alkenyl, C₂-C₃₀ alkynyl, C₃-C₃₀ aryl, C₃-C₃₀ heteroaryl, C₁-C₃₀ alkoxy, C₃-C₃₀ aryloxy, C₃-C₃₀ arylthio, C₁-C₃₀ alkylthio, C₂-C₃₀ carbonyl, C₁-C₃₀ carboxyl, amino, amido, or polyaryl (containing fused or non-fused ring moieties);
(c) g, h and i are independently 0 or a positive integer, such as 1, and g+h+i>0;
(d) Linker represents structures that facilitate the optional linking moieties between L and P/AA, wherein Linker is preferably selected from unsubstituted and substituted alkyl group, unsubstituted and substituted heteroalkyl group, unsubstituted and substituted alkenyl group, unsubstituted and substituted heteroalkenyl group, unsubstituted and substituted alkynyl group, unsubstituted and substituted heteroalkynyl group, unsubstituted and substituted aryl group, unsubstituted and substituted heteroaryl group, unsubstituted and substituted sulfonyl group, unsubstituted and substituted amide group, unsubstituted and substituted azo group, unsubstituted and substituted acyl group, unsubstituted and substituted ester group, unsubstituted and substituted carbonate group, unsubstituted and substituted ether group, unsubstituted and substituted aminooxy group, unsubstituted and substituted hydroxyamino group, and their derivatives, and a combination thereof,
(e) AA are selected from amino acids, their derivatives, or combination amino acids and/or their derivatives, preferably AA are selected from: 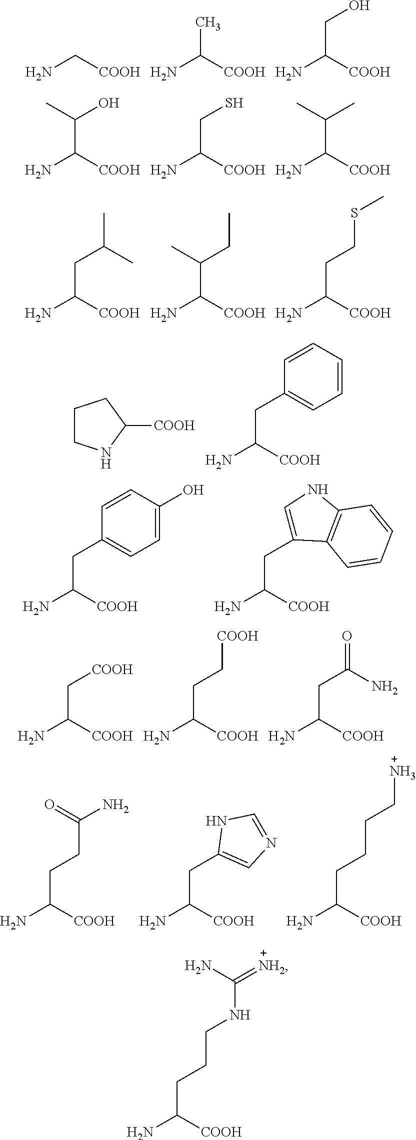
(f) P represents positive charge-containing structure(s), preferably substituted and unsubstituted amine, ammonium, pyridinium, pyrrodinium, phosphonium, imidazolium, sulfonium, and their derivatives;
(g) n+/− is a charged state of the compound, wherein n is zero or a positive integer, such as 1, 2, 3, 4, and 5;
(h) X is a counterion for charge neutrality of the compound, wherein when X^{m-/+} is an anion denoted as X^{m-}, X^{m-} is preferably selected from chloride (Cl⁻), hexafluorophosphate (PF₆⁻), nitrate (NO₃⁻), perchlorate (ClO₄⁻), tetrafluoroborate (BF₄⁻), tetraphenylborate (B(C₆H₅)₄⁻), triflate (CF₃SO₃⁻), dihydrogenphosphate (H₂PO₄²⁻), sulfate (SO₄²⁻), hydrogenphosphate (HPO₄²⁻), phosphate (PO₄⁻³), and derivatives thereof, wherein when X^{m-/+} is a cation denoted as X^{m+}, X^{m+} is preferably selected from K⁺, Na⁺, Ca²⁺, Mg²⁺, bis(triphenylphosphine)iminium ([(C₆H₅)₃P)₂N]⁺), phosphonium, pyridinium ([C₅H₅NH]⁺), quaternary ammonium cations, and derivatives thereof,
(i) m−/+ is a charged state of the counterion, wherein m is zero or a positive integer, such as 1, 2, 3, 4, and 5, wherein m=n or m≠n; and
(j) n/m represents a stoichiometry of the counterions in Formula VII.

11. The compound of claim 1, capable of binding to an analyte through noncovalent interactions such as electrostatic interactions, hydrogen bonding interactions, hydrophobic interaction, and combinations thereof.

12. The compound of claim 1, wherein the compound exhibits a square-planar, a trigonal planar, a partially planar, or a linear geometry.

13. The compound of claim 11, wherein the analyte is capable of inducing self-assembly of the compound, preferably wherein self-assembly leads to changes in the compound's photophysical properties, such as changes in the UV-vis absorbance, emission wavelength, emission intensity, emission lifetime, circular dichroism, circularly polarized luminescence, or combinations thereof, preferably wherein the changes are used to sense the analyte.

14. The compound of claim 1 having a structure: 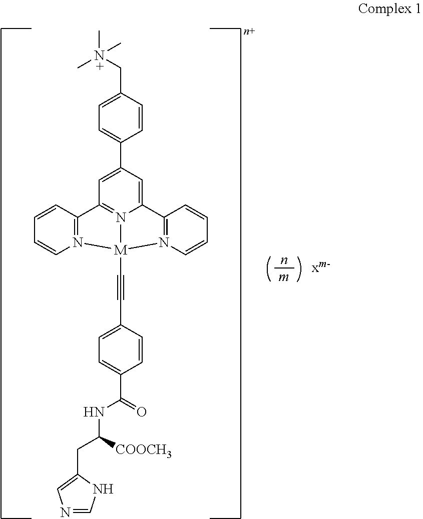
wherein M represents the metal center, preferably selected from Ir(I), Rh(I), Pt(II), Pd(II), Ni(II), Au(III), Ag(III), or Cu(III); 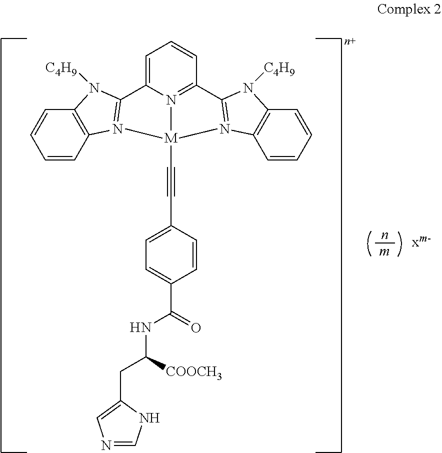
wherein M represents the metal center, preferably selected from Ir(I), Rh(I), Pt(II), Pd(II), Ni(II), Au(III), Ag(III), or Cu(III); 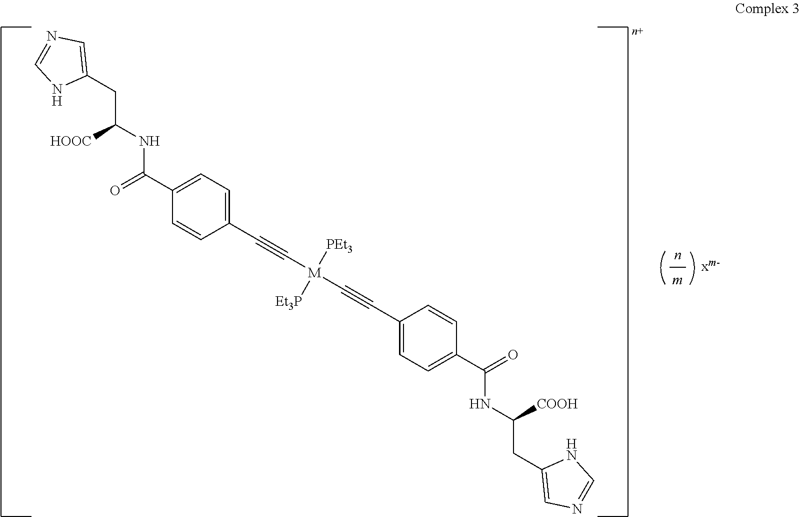
wherein M represents the metal center, preferably selected from Ir(I), Rh(I), Pt(II), Pd(II), Ni(II), Au(III), Ag(III), or Cu(III); 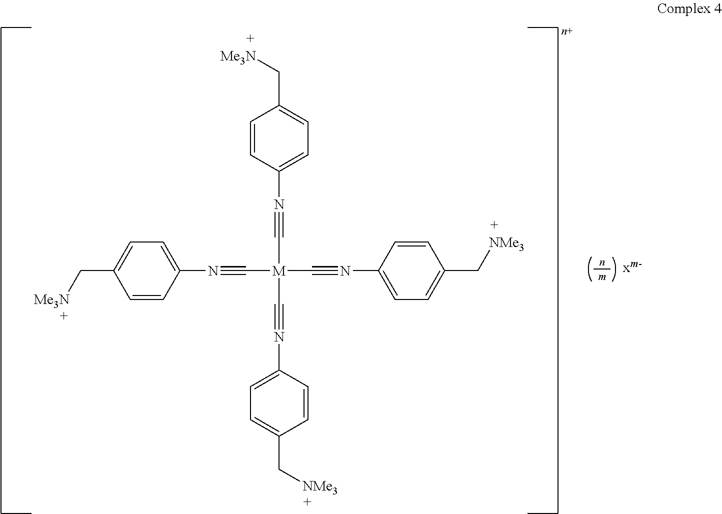
wherein M represents the metal center, preferably selected from Ir(I), Rh(I), Pt(II), Pd(II), Ni(II), Au(III), Ag(III), or Cu(III); 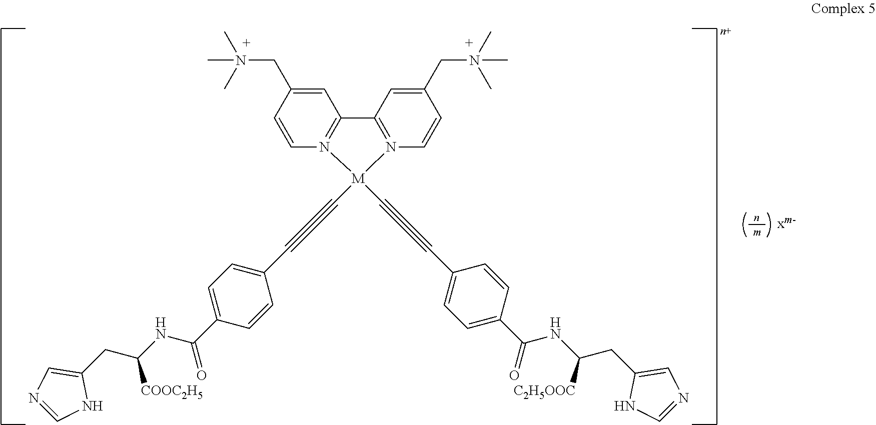
wherein M represents the metal center, preferably selected from Ir(I), Rh(I), Pt(II), Pd(II), Ni(II), Au(III), Ag(III), or Cu(III); 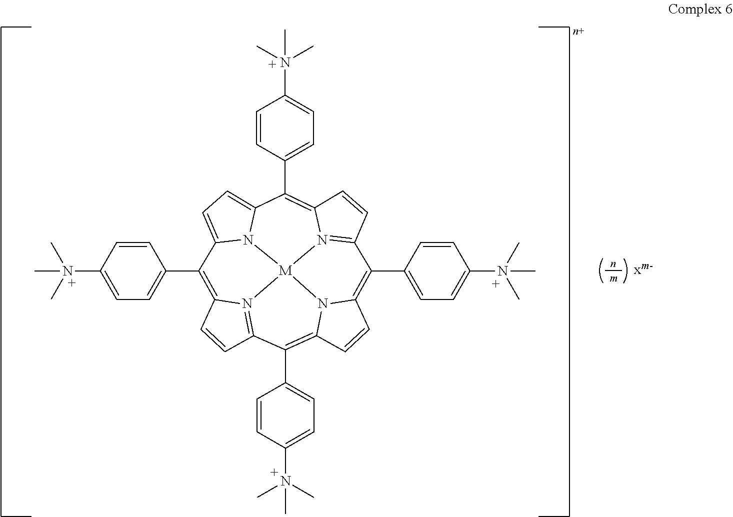
wherein M represents the metal center, preferably selected from Ir(I), Rh(I), Pt(II), Pd(II), Ni(II), Au(III), Ag(III), or Cu(III); 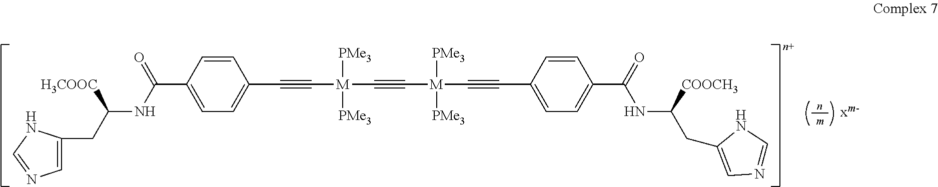
wherein M represents the metal center, preferably selected from Ir(I), Rh(I), Pt(II), Pd(II), Ni(II), Au(III), Ag(III), or Cu(III); 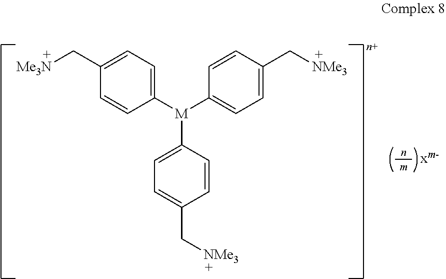
wherein M represents the metal center, preferably selected from Ir(I), Rh(I), Pt(II), Pd(II)t, Ni(II), Au(III), Ag(III), or Cu(III); 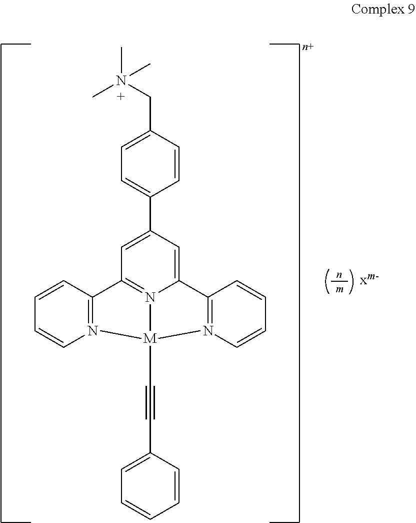
wherein M represents the metal center, preferably selected from Ir(I), Rh(I), Pt(II), Pd(II), Ni(II), Au(III), Ag(III), or Cu(III), n represents the number of charges of the metal complex in the formula, wherein n is zero or a positive integer, X represents the counterion for charge neutrality, wherein n is zero or a positive integer, m=n or m≠n, n/m stands for the stoichiometry of the counterions in the formula; 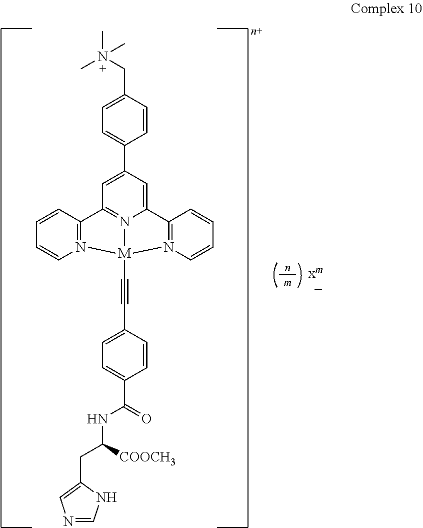
wherein M represents the metal center, preferably selected from Ir(I), Rh(I), Pt(II), Pd(II), Ni(II), Au(III), Ag(III), or Cu(III), n represents the number of charges of the metal complex in the formula, wherein n is zero or a positive integer, X represents the counterion for charge neutrality, wherein n is zero or a positive integer, m=n or m≠n, n/m stands for the stoichiometry of the counterions in the formula; 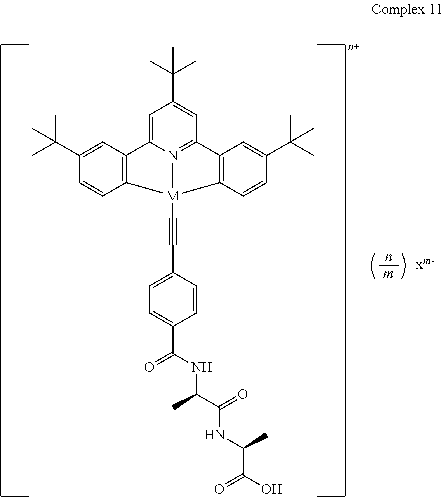
wherein M represents the metal center, preferably selected from Ir(I), Rh(I), Pt(II), Pd(II), Ni(II), Au(III), Ag(III), or Cu(III), n represents the number of charges of the metal complex in the formula, wherein n is zero or a positive integer, X represents the counterion for charge neutrality, wherein n is zero or a positive integer, m=n or m≠n, n/m stands for the stoichiometry of the counterions in the formula.

15. The compound of claim 11, wherein the analyte is a glycan, such as sialic acids or polysialic acids; and/or cancer cells.

16. A method for sensing or imaging analyte in a sample (preferably a biological sample), the method comprising: (a) combining the compound claim 1, preferably in a container, optionally followed by mixing the compound with the sample; and (b) measuring changes in the photophysical properties of the compound, and optionally checking whether the supramolecular self-assembly of the compound occurs, and preferably induced by combining the compound with the sample.

17. The method of claim 16, wherein a variation in the photophysical properties of the compound indicates changes in the supramolecular self-assembly and aggregation behavior of the compound, wherein the variation indicates presence of the analyte.

18. The method of claim 16, wherein the analyte that can be sensed or imaged comprises glycans such as sialic acids or polysialic acids; and/or cancer cells.

19. The method of claim 16, wherein the sample comprises body fluid (such as blood, plasma, serum), cells (such as eukaryotic cells optionally selected from 3T3 cells, HeLa cells, HepG2 cells, MCF7 cells, HEK293T cells, Chinese hamster ovary (CHO) cells and other cells), tissues (such as brain tissue, heart tissue, liver tissue, kidney tissue, spleen tissue, lung tissue, etc.), or animals.

20. A method for testing the efficacy of inhibitors to remove analytes or inhibit the generation of analytes, the method comprising: (1) mixing the compound of claim 1 with the inhibitor-treated samples and corresponding samples that are not treated with the inhibitor; (2) measuring the changes in the photophysical properties of the compound to investigate the changes in the degree of supramolecular self-assembly of the complex, preferably wherein, changes in the photophysical properties indicate the changes in the self-assembly and aggregation behavior of the compound.

21. A kit containing, in one or more containers, a compound of claim 1, and optionally positive controls, negative controls, and/or instructions for using the kit.

22. The kit of claim 21, wherein the compound is capable of detecting and/or imaging an analyte, wherein the analyte is selected from glycans such as sialic acids or polysialic acids; and/or cancer cells.
